SEQUENCE LISTING (194 sequences)

SEQ ID NO: 1 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCQDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 2 - protein, 468 aa, Homo sapiens
TQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEP

SEQ ID NO: 3 - protein, 79 aa, Homo sapiens
TQCIGEDGVQHQFLEAWVPDHQPCQICTCLSGRKVNCTTQPCPTAKAPTCGLCEVARLRQNADQCCPEYECVCDPVSCD

SEQ ID NO: 4 - protein, 69 aa, Homo sapiens
LPPVPHCERGLQPTLTNPGECRPNFTCACRKEECKRVSPPSCPPHRLPTLRKTQCCDEYECACNCVNST

SEQ ID NO: 5 - protein, 67 aa, Homo sapiens
VCVHRSTIYPVGQFWEEGCDVCTCTDMEDAVMGLRVAQCSQKPCEDSCRSGFTYVLHEGECCGRCLP

SEQ ID NO: 6 - protein, 81 aa, Homo sapiens
SACEVVTGSPRGDSQSSWKSVGSQWASPENPCLINECVRVKEEVFIQQRNVSCPQLEVPVCPSGFQLSCKTSACCPSCRCE

SEQ ID NO: 7 - protein, 69 aa, Homo sapiens
RMEACMLNGTVIGPGKTVMIDVCTTCRCMVQVGVISGFKLECRKTTCNPCPLGYKEENNTGECCGRCLP

SEQ ID NO: 8 - protein, 76 aa, Homo sapiens
TACTIQLRGGQIMTLKRDETLQDGCDTHFCKVNERGEYFWEKRVTGCPPFDEHKCLAEGGKIMKIPGTCCDTCEEP

SEQ ID NO: 9 - protein, 8 aa, Artificial Sequence
  1-A2 CDR-H1
GIDLTSNA

SEQ ID NO: 10 - protein, 7 aa, Artificial Sequence
  1-A2 CDR-H2
IYGHDTS

SEQ ID NO: 11 - protein, 9 aa, Artificial Sequence
  1-A2 CDR-H3
ARGFIYFDI

SEQ ID NO: 12 - protein, 25 aa, Artificial Sequence
  1-A2 FR-H1
SQSVEESGGRLVPPGTPLTLTCTVS

SEQ ID NO: 13 - protein, 17 aa, Artificial Sequence
  1-A2 FR-H2
MNWVRQAPGKGLEWIGG

SEQ ID NO: 14 - protein, 36 aa, Artificial Sequence
  1-A2 FR-H3
YYAAWAKGRFTISRTSTTVDLKMTRPTTDDTATYFC

SEQ ID NO: 15 - protein, 11 aa, Artificial Sequence
  1-A2 FR-H4
WGTGTLVTISS

SEQ ID NO: 16 - protein, 112 aa, Artificial Sequence
  1-A2 VH
QSVEESGGRLVPPGTPLTLTCTVSGIDLTSNAMNWVRQAPGKGLEWIGGIYGHDTSYYAAWAKGRFTISRTSTTVDLKMTRPTTDDTATYFCARGFIYFDIWGTGTLVTISS

SEQ ID NO: 17 - DNA, 336 bp, Artificial Sequence
  1-A2 VH
cagtcggtggaggagtccgggggtcgcctggtcccgcctgggacacccctgacactcacctgcacagtctctggaatcgacctcactagcaatgcaatgaactgggtccgccaggctccagggaaggggctggaatggatcggaggcatttatggtcatgatacctcatattacgcggcctgggcgaaaggccgattcaccatctccagaacctcgaccacagtggatctgaaaatgaccaggccgacaaccgacgacacggccacctatttctgtgccagaggttttatttattttgacatctggggcacaggcaccctggtcaccatctcttca

SEQ ID NO: 18 - protein, 6 aa, Artificial Sequence
  1-A2 CDR-L1
EDIYSG

SEQ ID NO: 19 - protein, 3 aa, Artificial Sequence
  1-A2 CDR-L2
GAS

SEQ ID NO: 20 - protein, 12 aa, Artificial Sequence
  1-A2 CDR-L3
LGGHSHSTTDLT

SEQ ID NO: 21 - protein, 26 aa, Artificial Sequence
  1-A2 FR-L1
AIEMTQTPPSLSASVGETVRIRCLAS

SEQ ID NO: 22 - protein, 17 aa, Artificial Sequence
  1-A2 FR-L2
ISWYQQKPGKPPTLLIY

SEQ ID NO: 23 - protein, 36 aa, Artificial Sequence
  1-A2 FR-L3
NLESGVPPRFSGSGSGTDYTLTIGGVQAEDAATYYC

SEQ ID NO: 24 - protein, 10 aa, Artificial Sequence
  1-A2 FR-L4
FGAGTKVEIK

SEQ ID NO: 25 - protein, 110 aa, Artificial Sequence
  1-A2 VL
AIEMTQTPPSLSASVGETVRIRCLASEDIYSGISWYQQKPGKPPTLLIYGASNLESGVPPRFSGSGSGTDYTLTIGGVQAEDAATYYCLGGHSHSTTDLTFGAGTKVEIK

SEQ ID NO: 26 - DNA, 330 bp, Artificial Sequence
  1-A2 VL
gcaattgagatgacccagactccaccctccctgtctgcatctgtgggagaaactgtcaggattaggtgcctggccagtgaggacatttacagtggtatatcctggtatcaacagaagccagggaaacctcctacactcctgatctatggtgcatccaatttagaatctggggtcccaccacggttcagtggcagtggatctgggacagattacaccctcaccattggcggcgtgcaggctgaagatgctgccacctactactgtctaggcggtcatagccacagtactaccgatttgacttttggagctgggaccaaggtggaaatcaaa

SEQ ID NO: 27 - protein, 8 aa, Artificial Sequence
  4-H3 CDR-H1
GIDLTSNA

SEQ ID NO: 28 - protein, 7 aa, Artificial Sequence
  4-H3 CDR-H2
IYGHDTS

SEQ ID NO: 29 - protein, 9 aa, Artificial Sequence
  4-H3 CDR-H3
ARGFIYFDI

SEQ ID NO: 30 - protein, 25 aa, Artificial Sequence
  4-H3 FR-H1
SQSLEESGGRLVPPGTPLTLTCTVS

SEQ ID NO: 31 - protein, 17 aa, Artificial Sequence
  4-H3 FR-H2
MNWVRQAPGKGLEWIGG

SEQ ID NO: 32 - protein, 36 aa, Artificial Sequence; identical to SEQ ID NO: 14
  4-H3 FR-H3

SEQ ID NO: 33 - protein, 11 aa, Artificial Sequence
  4-H3 FR-H4
WGTGTLVTISS

SEQ ID NO: 34 - protein, 112 aa, Artificial Sequence
  4-H3 VH
QSLEESGGRLVPPGTPLTLTCTVSGIDLTSNAMNWVRQAPGKGLEWIGGIYGHDTSYYAAWAKGRFTISRTSTTVDLKMTRPTTDDTATYFCARGFIYFDIWGTGTLVTISS

SEQ ID NO: 35 - DNA, 336 bp, Artificial Sequence
  4-H3 VH
cagtcgctggaggagtccgggggtcgcctggtcccgcctgggacacccctgacactcacctgcacagtctctggaatcgacctcactagcaatgcaatgaactgggtccgccaggctccagggaaggggctggaatggatcggaggcatttatggtcatgatacctcatattacgcggcctgggcgaaaggccgattcaccatctccagaacctcgaccacagtggatctgaaaatgaccaggccgacaaccgacgacacggccacctatttctgtgccagaggttttatttattttgacatctggggcacaggcaccctggtcaccatctcttca

SEQ ID NO: 36 - protein, 6 aa, Artificial Sequence
  4-H3 CDR-L1
EDIASG

SEQ ID NO: 37 - protein, 3 aa, Artificial Sequence
  4-H3 CDR-L2
GAS

SEQ ID NO: 38 - protein, 12 aa, Artificial Sequence
  4-H3 CDR-L3
LGGYSFSSNGLT

SEQ ID NO: 39 - protein, 26 aa, Artificial Sequence
  4-H3 FR-L1
AYDMTQTPPSLSASVGETVRIRCLAS

SEQ ID NO: 40 - protein, 17 aa, Artificial Sequence
  4-H3 FR-L2
ISWYQQKPGKPPTLLIY

SEQ ID NO: 41 - protein, 36 aa, Artificial Sequence; identical to SEQ ID NO: 23
  4-H3 FR-L3

SEQ ID NO: 42 - protein, 10 aa, Artificial Sequence
  4-H3 FR-L4
FGAGTKVEIK

SEQ ID NO: 43 - protein, 110 aa, Artificial Sequence
  4-H3 VL
AYDMTQTPPSLSASVGETVRIRCLASEDIASGISWYQQKPGKPPTLLIYGASNLESGVPPRFSGSGSGTDYTLTIGGVQAEDAATYYCLGGYSFSSNGLTFGAGTKVEIK

SEQ ID NO: 44 - DNA, 330 bp, Artificial Sequence
  4-H3 VL
gcttatgatatgacccagactccaccctccctgtctgcatctgtgggagaaactgtcaggattaggtgcctggccagtgaggacattgccagtggtatatcctggtatcaacagaagccagggaaacctcctacactcctgatctatggtgcatccaatttagaatctggggtcccaccacggttcagtggcagtggatctgggacagattacaccctcaccattggcggcgtgcaggctgaagatgctgccacctactactgtctaggcggttatagtttcagtagtaacggtttgacttttggagctggcaccaaggtggagatcaaa

SEQ ID NO: 45 - protein, 8 aa, Artificial Sequence
  1-D5 CDR-H1
GFSLNNYI

SEQ ID NO: 46 - protein, 7 aa, Artificial Sequence
  1-D5 CDR-H2
ISTGGST

SEQ ID NO: 47 - protein, 13 aa, Artificial Sequence
  1-D5 CDR-H3
ARGGSSAGAGFNI

SEQ ID NO: 48 - protein, 25 aa, Artificial Sequence
  1-D5 FR-H1
QQQLVESGGRLVTPGTPLTLTCAVS

SEQ ID NO: 49 - protein, 17 aa, Artificial Sequence
  1-D5 FR-H2
MGWVRQAPGKGLEYIGI

SEQ ID NO: 50 - protein, 36 aa, Artificial Sequence
  1-D5 FR-H3
YYASWAKGRFTISRTSTTMDLKMTSLTTEDTATYFC

SEQ ID NO: 51 - protein, 11 aa, Artificial Sequence
  1-D5 FR-H4
WGPGTLVTVSS

SEQ ID NO: 52 - protein, 117 aa, Artificial Sequence
  1-D5 VH
QQQLVESGGRLVTPGTPLTLTCAVSGFSLNNYIMGWVRQAPGKGLEYIGIISTGGSTYYASWAKGRFTISRTSTTMDLKMTSLTTEDTATYFCARGGSSAGAGFNIWGPGTLVTVSS

SEQ ID NO: 53 - DNA, 351 bp, Artificial Sequence
  1-D5 VH
cagcagcagctggtggagtccgggggtcgcctggtcacgcctgggacacccctgacactaacctgcgcagtctctggattttccctcaataactacatcatgggctgggtccgccaggctccagggaaggggctggaatacatcggaatcattagtactggtggtagcacatactacgcgagctgggcaaaaggccgattcaccatctccagaacctcgaccacgatggatctgaaaatgaccagtctgacaaccgaggacacggccacctatttctgtgccagagggggtagtagtgctggtgcgggatttaatatctggggcccgggcaccctggtcaccgtctcctca

SEQ ID NO: 54 - protein, 6 aa, Artificial Sequence
  1-D5 CDR-L1
QSINSG

SEQ ID NO: 55 - protein, 3 aa, Artificial Sequence
  1-D5 CDR-L2
KAS

SEQ ID NO: 56 - protein, 12 aa, Artificial Sequence
  1-D5 CDR-L3
QSYHYISANGAT

SEQ ID NO: 57 - protein, 26 aa, Artificial Sequence
  1-D5 FR-L1
DIVMTQTPSSVSAAVGDTVTIQCQAS

SEQ ID NO: 58 - protein, 17 aa, Artificial Sequence
  1-D5 FR-L2
LAWYQQKPGQPPKRLIY

SEQ ID NO: 59 - protein, 36 aa, Artificial Sequence
  1-D5 FR-L3
TLASGVPSRFRGSGSGTDFTLTISDLECADAATYYC

SEQ ID NO: 60 - protein, 10 aa, Artificial Sequence
  1-D5 FR-L4
FGGGTEVVVE

SEQ ID NO: 61 - protein, 110 aa, Artificial Sequence
  1-D5 VL
DIVMTQTPSSVSAAVGDTVTIQCQASQSINSGLAWYQQKPGQPPKRLIYKASTLASGVPSRFRGSGSGTDFTLTISDLECADAATYYCQSYHYISANGATFGGGTEVVVE

SEQ ID NO: 62 - DNA, 330 bp, Artificial Sequence
  1-D5 VL
gatattgttatgacccagactccctcctccgtgtctgcagctgtgggagacacagtcaccatccagtgccaggccagtcagagcattaatagtggtttggcctggtatcagcagaaaccagggcagcctcccaagcgcctgatctacaaggcatccactctggcatctggggtcccatcgcggttcagaggcagtggatctgggacagacttcactctcaccatcagcgacctggagtgtgccgatgctgccacttactactgtcaaagctatcattatattagtgctaatggtgctactttcggcggagggaccgaggtggtcgtcgaa

SEQ ID NO: 63 - protein, 8 aa, Artificial Sequence
  3-H9 CDR-H1
GFSLSNYD

SEQ ID NO: 64 - protein, 7 aa, Artificial Sequence
  3-H9 CDR-H2
IHAIGIT

SEQ ID NO: 65 - protein, 9 aa, Artificial Sequence
  3-H9 CDR-H3
ARGLVDLNM

SEQ ID NO: 66 - protein, 25 aa, Artificial Sequence
  3-H9 FR-H1
SQSLEESGGRLVTPGTPLTLTCSVS

SEQ ID NO: 67 - protein, 17 aa, Artificial Sequence
  3-H9 FR-H2
MSWVRQAPGKGLEWIGS

SEQ ID NO: 68 - protein, 36 aa, Artificial Sequence
  3-H9 FR-H3
YYANWAEGRFTISKTSTTVDLKMTSLTTEDTATYFC

SEQ ID NO: 69 - protein, 11 aa, Artificial Sequence
  3-H9 FR-H4
WGPGTLVTVSS

SEQ ID NO: 70 - protein, 112 aa, Artificial Sequence
  3-H9 VH
QSLEESGGRLVTPGTPLTLTCSVSGFSLSNYDMSWVRQAPGKGLEWIGSIHAIGITYYANWAEGRFTISKTSTTVDLKMTSLTTEDTATYFCARGLVDLNMWGPGTLVTVSS

SEQ ID NO: 71 - DNA, 336 bp, Artificial Sequence
  3-H9 VH
cagtcgctggaggagtccgggggtcgcctggtcacgcctgggacacccttgacactcacctgttcagtctctggattctccctcagcaactacgacatgagctgggtccgccaggctccagggaagggactggaatggatcgggtccatacatgctattggtatcacatactacgcgaactgggcggaaggccgattcaccatctccaaaacctcgaccacggtggatctgaaaatgaccagtctgacaaccgaggacacggccacctatttctgtgccagagggctggtagatttgaacatgtggggcccgggcaccctcgtcactgtctcttca

SEQ ID NO: 72 - protein, 8 aa, Artificial Sequence
  3-H9 CDR-L1
QSVYSNNL

SEQ ID NO: 73 - protein, 3 aa, Artificial Sequence
  3-H9 CDR-L2
DAS

SEQ ID NO: 74 - protein, 12 aa, Artificial Sequence
  3-H9 CDR-L3
QGSYYSSGWYNT

SEQ ID NO: 75 - protein, 26 aa, Artificial Sequence
  3-H9 FR-L1
AIKMTQTPSSVSVAVGGTVTINCQSS

SEQ ID NO: 76 - protein, 17 aa, Artificial Sequence
  3-H9 FR-L2
LSWYQQKPGQPPKLLIY

SEQ ID NO: 77 - protein, 36 aa, Artificial Sequence
  3-H9 FR-L3
TLESGVPSRFKGSGSGTQFTLTISGVQCEDAATYYC

SEQ ID NO: 78 - protein, 10 aa, Artificial Sequence
  3-H9 FR-L4
FGGGTEVVVE

SEQ ID NO: 79 - protein, 112 aa, Artificial Sequence
  3-H9 VL
AIKMTQTPSSVSVAVGGTVTINCQSSQSVYSNNLLSWYQQKPGQPPKLLIYDASTLESGVPSRFKGSGSGTQFTLTISGVQCEDAATYYCQGSYYSSGWYNTFGGGTEVVVE

SEQ ID NO: 80 - DNA, 336 bp, Artificial Sequence
  3-H9 VL
gctattaaaatgacccagactccatcgtccgtgtctgtagctgtgggaggcacagtcaccatcaattgccagtccagtcagagtgtttatagtaacaacctcttatcttggtaccagcagaaaccagggcagcctcccaagctcttgatctacgatgcatccactctggaatctggggtcccatcgcggttcaaaggcagtggatctgggacacagttcactctcaccatcagcggcgtgcagtgtgaggatgctgccacttactactgtcaaggcagttattatagtagtggttggtacaatactttcggcggagggaccgaggtggtcgtcgaa

SEQ ID NO: 81 - protein, 8 aa, Artificial Sequence
  1-G5 CDR-H1
GFSLSSYD

SEQ ID NO: 82 - protein, 7 aa, Artificial Sequence
  1-G5 CDR-H2
IHATGIT

SEQ ID NO: 83 - protein, 9 aa, Artificial Sequence
  1-G5 CDR-H3
ARGLVDLNM

SEQ ID NO: 84 - protein, 25 aa, Artificial Sequence
  1-G5 FR-H1
SQSLEESGGRLVTPGTPLTLTCSVS

SEQ ID NO: 85 - protein, 17 aa, Artificial Sequence
  1-G5 FR-H2
MTWVRQAPGKGLEWIGS

SEQ ID NO: 86 - protein, 36 aa, Artificial Sequence
  1-G5 FR-H3
FYANWAKGRFTTSKTSTTVDLKMTSLTTEDTATYFC

SEQ ID NO: 87 - protein, 11 aa, Artificial Sequence
  1-G5 FR-H4
WGPGTLVTVSS

SEQ ID NO: 88 - protein, 112 aa, Artificial Sequence
  1-G5 VH
QSLEESGGRLVTPGTPLTLTCSVSGFSLSSYDMTWVRQAPGKGLEWIGSIHATGITFYANWAKGRFTTSKTSTTVDLKMTSLTTEDTATYFCARGLVDLNMWGPGTLVTVSS

SEQ ID NO: 89 - DNA, 336 bp, Artificial Sequence
  1-G5 VH
cagtcgttggaggagtccgggggtcgcctggtcacgcctgggacacccttgacactcacctgttcagtctctggattctccctcagcagctacgacatgacctgggtccgccaggctccagggaaggggctggaatggatcgggtccatacatgctactggtatcacattctacgcgaactgggcgaaaggccgattcaccacctccaaaacctcgaccacggtggatctgaaaatgaccagtctgacaaccgaggacacggccacctatttctgtgccagagggctggtagatttgaacatgtggggcccgggcaccctcgtcaccgtctcttca

SEQ ID NO: 90 - protein, 8 aa, Artificial Sequence
  1-G5 CDR-L1
QSVYNNNY

SEQ ID NO: 91 - protein, 3 aa, Artificial Sequence
  1-G5 CDR-L2
DAS

SEQ ID NO: 92 - protein, 12 aa, Artificial Sequence
  1-G5 CDR-L3
QGSYYSGGWDTA

SEQ ID NO: 93 - protein, 26 aa, Artificial Sequence
  1-G5 FR-L1
DPVMTQTASSVSAAVGGTVTINCQAS

SEQ ID NO: 94 - protein, 17 aa, Artificial Sequence
  1-G5 FR-L2
LSWYQQKPGQPPKLLIY

SEQ ID NO: 95 - protein, 36 aa, Artificial Sequence
  1-G5 FR-L3
TLASGVPSRFSGNGSGTQFTLTISGVQCDDAATYYC

SEQ ID NO: 96 - protein, 10 aa, Artificial Sequence
  1-G5 FR-L4
FGGGTKVVVK

SEQ ID NO: 97 - protein, 112 aa, Artificial Sequence
  1-G5 VL
DPVMTQTASSVSAAVGGTVTINCQASQSVYNNNYLSWYQQKPGQPPKLLIYDASTLASGVPSRFSGNGSGTQFTLTISGVQCDDAATYYCQGSYYSGGWDTAFGGGTKVVVK

SEQ ID NO: 98 - DNA, 336 bp, Artificial Sequence
  1-G5 VL
gatcccgtgatgacccagactgcgtcctccgtgtctgcagctgtgggaggcacagtcaccatcaattgccaggccagtcagagtgtttataataacaactacttatcctggtatcagcagaaaccagggcagcctcccaagctcttgatctacgatgcatccactctggcatctggggtcccatcccggttcagcggcaatggatctgggacacagttcactctcaccatcagcggcgtacagtgtgacgatgctgccacttactactgtcaaggcagttattatagtggtggttgggacactgctttcggcggagggaccaaggtggtcgtcaaa

SEQ ID NO: 99 - protein, 8 aa, Artificial Sequence
  4-H9 CDR-H1
GFSLNSFA

SEQ ID NO: 100 - protein, 7 aa, Artificial Sequence
  4-H9 CDR-H2
ITVDGHT

SEQ ID NO: 101 - protein, 17 aa, Artificial Sequence
  4-H9 CDR-H3
AREDAGDAGYIYATYNI

SEQ ID NO: 102 - protein, 25 aa, Artificial Sequence
  4-H9 FR-H1
SQSVEESGGRLVTPGTPLTLTCTAS

SEQ ID NO: 103 - protein, 17 aa, Artificial Sequence
  4-H9 FR-H2
MSWVRQAPGKGLEWIGI

SEQ ID NO: 104 - protein, 36 aa, Artificial Sequence
  4-H9 FR-H3
YYASWAKGRFTISKASTTVDLKITSPTTEDTATYFC

SEQ ID NO: 105 - protein, 11 aa, Artificial Sequence
  4-H9 FR-H4
WGPGTLVTVSS

SEQ ID NO: 106 - protein, 120 aa, Artificial Sequence
  4-H9 VH
QSVEESGGRLVTPGTPLTLTCTASGFSLNSFAMSWVRQAPGKGLEWIGIITVDGHTYYASWAKGRFTISKASTTVDLKITSPTTEDTATYFCAREDAGDAGYIYATYNIWGPGTLVTVSS

SEQ ID NO: 107 - DNA, 360 bp, Artificial Sequence
  4-H9 VH
caatcggtggaggagtccgggggtcgcctggtcacgcctgggacacccctgacactcacctgcacagcctctggattctccctcaatagctttgcgatgagctgggtccgccaggctccagggaaggggctggaatggatcggaatcattactgttgatggtcacacatactacgcgagctgggcgaaaggccgattcaccatctccaaagcctcgaccacggtggatctgaaaatcaccagtccgacaaccgaggacacggccacctatttctgtgccagagaggatgctggtgatgctggttatatttatgctacctataacatctggggcccagggaccctcgtcaccgtctcttca

SEQ ID NO: 108 - protein, 6 aa, Artificial Sequence
  4-H9 CDR-L1
EDIGYG

SEQ ID NO: 109 - protein, 3 aa, Artificial Sequence
  4-H9 CDR-L2
GAN

SEQ ID NO: 110 - protein, 9 aa, Artificial Sequence
  4-H9 CDR-L3
QQGYSTPPT

SEQ ID NO: 111 - protein, 26 aa, Artificial Sequence
  4-H9 FR-L1
AIEMTQTPSSLAASVGDTVTITCKAS

SEQ ID NO: 112 - protein, 17 aa, Artificial Sequence
  4-H9 FR-L2
LAWYQQKLGIAPKLLIY

SEQ ID NO: 113 - protein, 36 aa, Artificial Sequence
  4-H9 FR-L3
TLESGVPSRFSGSGSETDYTLTISSVQAEDAGIYYC

SEQ ID NO: 114 - protein, 10 aa, Artificial Sequence
  4-H9 FR-L4
FGAGTMVEIQ

SEQ ID NO: 115 - protein, 107 aa, Artificial Sequence
  4-H9 VL
AIEMTQTPSSLAASVGDTVTITCKASEDIGYGLAWYQQKLGIAPKLLIYGANTLESGVPSRFSGSGSETDYTLTISSVQAEDAGIYYCQQGYSTPPTFGAGTMVEIQ

SEQ ID NO: 116 - DNA, 321 bp, Artificial Sequence
  4-H9 VL
gcgattgaaatgacccagactccatcctccctggctgcatctgtgggagacacagtcaccatcacttgtaaggccagtgaggacattggttatgggttagcctggtatcagcagaaactggggatagctcctaagctcctgatctatggggcaaacactttagaatctggggtcccatcgaggttcagtggcagcggatcagagaccgattacaccctcaccatcagcagcgtgcaggctgaagatgcaggaatttattactgtcagcaaggatatagtacccctcctactttcggtgcggggaccatggtggagatccaa

SEQ ID NO: 117 - protein, 8 aa, Artificial Sequence
  4-B12 CDR-H1
GFSLNSFA

SEQ ID NO: 118 - protein, 7 aa, Artificial Sequence
  4-B12 CDR-H2
ITVDGHT

SEQ ID NO: 119 - protein, 17 aa, Artificial Sequence
  4-B12 CDR-H3
AREDAGDAGYIYATYNI

SEQ ID NO: 120 - protein, 25 aa, Artificial Sequence
  4-B12 FR-H1
SQSVKESEGRLVTPGTPLTLTCTVS

SEQ ID NO: 121 - protein, 17 aa, Artificial Sequence
  4-B12 FR-H2
MSWVRQAPGKGLEWIGI

SEQ ID NO: 122 - protein, 36 aa, Artificial Sequence
  4-B12 FR-H3
YYANWAKDRFTISKASTTVDLKITSPTTEDTATYFC

SEQ ID NO: 123 - protein, 11 aa, Artificial Sequence
  4-B12 FR-H4
WGPGTLVTVSS

SEQ ID NO: 124 - protein, 120 aa, Artificial Sequence
  4-B12 VH
QSVKESEGRLVTPGTPLTLTCTVSGFSLNSFAMSWVRQAPGKGLEWIGIITVDGHTYYANWAKDRFTISKASTTVDLKITSPTTEDTATYFCAREDAGDAGYIYATYNIWGPGTLVTVSS

SEQ ID NO: 125 - DNA, 360 bp, Artificial Sequence
  4-B12 VH
cagtcggtgaaggagtccgagggtcgcctggtcacgcctgggacacccctgacactcacctgcacagtctctggattctccctcaatagctttgcgatgagctgggtccgccaggctccagggaaggggctggaatggatcggaatcataactgttgatggtcacacatactacgcgaactgggcgaaagaccgattcaccatctccaaagcctcgaccacggtggatctgaaaatcaccagtccgacaaccgaggacacggccacctatttctgtgccagagaggatgctggtgatgctggttatatttatgctacctataacatctggggcccgggcaccctggtcaccgtctcctca

SEQ ID NO: 126 - protein, 6 aa, Artificial Sequence
  4-B12 CDR-L1
EDIGYG

SEQ ID NO: 127 - protein, 3 aa, Artificial Sequence
  4-B12 CDR-L2
GAN

SEQ ID NO: 128 - protein, 9 aa, Artificial Sequence
  4-B12 CDR-L3
QQGYSTPPT

SEQ ID NO: 129 - protein, 26 aa, Artificial Sequence
  4-B12 FR-L1
DPVLTQTASSLAASVGDTVTITCKAS

SEQ ID NO: 130 - protein, 17 aa, Artificial Sequence
  4-B12 FR-L2
LAWYQQKPGQPPKLLIY

SEQ ID NO: 131 - protein, 36 aa, Artificial Sequence
  4-B12 FR-L3
TLESGVPSRFTGSGSETDYTLTISSVQAEDAGIYYC

SEQ ID NO: 132 - protein, 10 aa, Artificial Sequence
  4-B12 FR-L4
FGAGTKVEIK

SEQ ID NO: 133 - protein, 107 aa, Artificial Sequence
  4-B12 VL
DPVLTQTASSLAASVGDTVTITCKASEDIGYGLAWYQQKPGQPPKLLIYGANTLESGVPSRFTGSGSETDYTLTISSVQAEDAGIYYCQQGYSTPPTFGAGTKVEIK

SEQ ID NO: 134 - DNA, 321 bp, Artificial Sequence
  4-B12 VL
gatcctgtgctgacccagactgcgtcctccctggctgcatctgtgggagacacagtcaccatcacttgtaaggccagtgaggacattggttatgggttagcctggtatcagcagaaaccagggcagcctcccaagctcctgatctatggggcaaacactttagaatctggggtcccatcgaggttcactggcagcggatcagagaccgattacaccctcaccatcagcagcgtgcaggctgaagatgcaggaatttattactgtcagcaaggatatagtacccctcctactttcggtgcgggcaccaaggtagaaatcaaa

SEQ ID NO: 135 - protein, 8 aa, Artificial Sequence
  4-C6 CDR-H1
GFSLNTYV

SEQ ID NO: 136 - protein, 7 aa, Artificial Sequence
  4-C6 CDR-H2
INGDSNT

SEQ ID NO: 137 - protein, 17 aa, Artificial Sequence
  4-C6 CDR-H3
AREDAADAGYVYATYNI

SEQ ID NO: 138 - protein, 25 aa, Artificial Sequence
  4-C6 FR-H1
SQSLEESGGRLVTPGTPLTLTCTAS

SEQ ID NO: 139 - protein, 17 aa, Artificial Sequence
  4-C6 FR-H2
MTWVRQAPGKGLEWIGF

SEQ ID NO: 140 - protein, 36 aa, Artificial Sequence
  4-C6 FR-H3
YYANWAKGRFTISKTSTTVDLKITSPTTEDTATYFC

SEQ ID NO: 141 - protein, 11 aa, Artificial Sequence
  4-C6 FR-H4
WGTGTLVTISS

SEQ ID NO: 142 - protein, 120 aa, Artificial Sequence
  4-C6 VH
QSLEESGGRLVTPGTPLTLTCTASGFSLNTYVMTWVRQAPGKGLEWIGFINGDSNTYYANWAKGRFTISKTSTTVDLKITSPTTEDTATYFCAREDAADAGYVYATYNIWGTGTLVTISS

SEQ ID NO: 143 - DNA, 360 bp, Artificial Sequence
  4-C6 VH
cagtcgctggaggagtccgggggtcgcctggtcacgcctgggacacccctgacactcacctgcacagcctctggattctccctcaatacctatgtaatgacctgggtccgccaggctccagggaaggggctggaatggatcggattcattaatggtgatagtaacacatactacgcgaactgggcgaaaggccgattcaccatctccaaaacctcgaccacggtggatctgaaaatcaccagtccgacaaccgaggacacggccacctatttctgtgccagagaggatgctgctgatgctggttatgtttatgctacctataacatctggggcacaggcaccctggtcaccatctcttca

SEQ ID NO: 144 - protein, 6 aa, Artificial Sequence
  4-C6 CDR-L1
EDIGYG

SEQ ID NO: 145 - protein, 3 aa, Artificial Sequence
  4-C6 CDR-L2
GAN

SEQ ID NO: 146 - protein, 9 aa, Artificial Sequence
  4-C6 CDR-L3
QQGYSTPPT

SEQ ID NO: 147 - protein, 26 aa, Artificial Sequence
  4-C6 FR-L1
AYDMTQTPSSLAASVGDTVTITCKAS

SEQ ID NO: 148 - protein, 17 aa, Artificial Sequence
  4-C6 FR-L2
LNWYQQKLGIAPKLLIY

SEQ ID NO: 149 - protein, 36 aa, Artificial Sequence; identical to SEQ ID NO: 113
  4-C6 FR-L3

SEQ ID NO: 150 - protein, 10 aa, Artificial Sequence
  4-C6 FR-L4
FGAGTMVEIK

SEQ ID NO: 151 - protein, 107 aa, Artificial Sequence
  4-C6 VL
AYDMTQTPSSLAASVGDTVTITCKASEDIGYGLNWYQQKLGIAPKLLIYGANTLESGVPSRFSGSGSETDYTLTISSVQAEDAGIYYCQQGYSTPPTFGAGTMVEIK

SEQ ID NO: 152 - DNA, 321 bp, Artificial Sequence
  4-C6 VL
gcatatgatatgacccagactccatcctccctggctgcatctgtgggagacacagtcaccatcacttgtaaggccagtgaggacattggttatgggttgaactggtatcagcagaaactagggatagctcctaagctcctcatctatggggcaaacactttagaatccggggtcccatcgaggttcagtggcagcggatcagagaccgattacaccctcaccatcagcagcgtgcaggctgaagatgcaggaatttattactgtcagcaaggatatagtacccctcctactttcggtgcggggaccatggtggagatcaaa

SEQ ID NO: 153 - protein, 177 aa, Homo sapiens
DLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEI

SEQ ID NO: 154 - protein, 172 aa, Homo sapiens
DVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVQRCC

SEQ ID NO: 155 - protein, 181 aa, Homo sapiens
DVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKL

SEQ ID NO: 156 - protein, 113 aa, Artificial Sequence
  1-A2 VH
SQSVEESGGRLVPPGTPLTLTCTVSGIDLTSNAMNWVRQAPGKGLEWIGGIYGHDTSYYAAWAKGRFTISRTSTTVDLKMTRPTTDDTATYFCARGFIYFDIWGTGTLVTISS

SEQ ID NO: 157 - protein, 330 aa, Artificial Sequence [sequence omitted]

SEQ ID NO: 158 - protein, 106 aa, Artificial Sequence
  mAb LC
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 113 aa, Artificial Sequence
  4-H3 VH
SQSLEESGGRLVPPGTPLTLTCTVSGIDLTSNAMNWVRQAPGKGLEWIGGIYGHDTSYYAAWAKGRFTISRTSTTVDLKMTRPTTDDTATYFCARGFIYFDIWGTGTLVTISS

SEQ ID NO: 160 - protein, 113 aa, Artificial Sequence
  3-H9 VH
SQSLEESGGRLVTPGTPLTLTCSVSGFSLSNYDMSWVRQAPGKGLEWIGSIHAIGITYYANWAEGRFTISKTSTTVDLKMTSLTTEDTATYFCARGLVDLNMWGPGTLVTVSS

SEQ ID NO: 161 - protein, 113 aa, Artificial Sequence
  1-G5 VH
SQSLEESGGRLVTPGTPLTLTCSVSGFSLSSYDMTWVRQAPGKGLEWIGSIHATGITFYANWAKGRFTTSKTSTTVDLKMTSLTTEDTATYFCARGLVDLNMWGPGTLVTVSS

SEQ ID NO: 162 - protein, 330 aa, Artificial Sequence
  mAb HC
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 107 aa, Artificial Sequence
  mAb HC
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPSCGGGSK

SEQ ID NO: 164 - protein, 8 aa, Artificial Sequence
  1-A2 and 4-H3 CDR-H1
GFTFSSNA

SEQ ID NO: 165 - protein, 115 aa, Artificial Sequence
  4-H3 and 1-A2 VH
QVQLVESGGGVVQPGRSLRLSCAASGFTFSSNAMNWVRQAPGKGLEWVAGIYGHDTSYYAAWAKGRFTISRDNSKNTLYLQMNSLRAEDTAVYYCARGFIYFDIWGQGTLVTVSS

SEQ ID NO: 166 - protein, 113 aa, Artificial Sequence
  4-H3 VH
QVQLVESGGGVVQPGRSLRLSCAASGIDLTSNAMNWVRQAPGKGLEWVAGIYGHDTSYYAAWAKGRFTISRDSNTLYLQMNSLRAEDTAVYYCARGFIYFDIWGQGTLVTVSS

SEQ ID NO: 167 - protein, 115 aa, Artificial Sequence
  4-H3 VH
QVQLVESGGGVVQPGRSLRLSCAASGIDLTSNAMNWVRQAPGKGLEWIGGIYGHDTSYYAAWAKGRFTISRDNSKNTVYLQMNSLRAEDTAVYYCARGFIYFDIWGQGTLVTVSS

SEQ ID NO: 168 - protein, 115 aa, Artificial Sequence
  4-H3 VH
QVQLVESGGGVVQPGRSLRLSCAASGIDLTSNAMNWVRQAPGKGLEWVAGIYGHDTSYYAAWAKGRFTISRDNSKNTLYLQMNSLRAEDTATYFCARGFIYFDIWGQGTLVTVSS

SEQ ID NO: 169 - protein, 114 aa, Artificial Sequence
  4-H3 VH
QQLVESGGGVVQPGRSLRLSCAASGIDLTSNAMNWVRQAPGKGLEWVAGIYGHDTSYYAAWAKGRFTISRDNSKNTLYLQMNSLRAEDTAVYYCARGFIYFDIWGQGTLVTVSS

SEQ ID NO: 170 - protein, 112 aa, Artificial Sequence
  4-H3 VH
QQLVESGGGVVQPGRSLRLSCAASGIDLTSNAMNWVRQAPGKGLEWVAGIYGHDTSYYAAWAKGRFTISRDSNTLYLQMNSLRAEDTAVYYCARGFIYFDIWGQGTLVTVSS

SEQ ID NO: 171 - protein, 115 aa, Artificial Sequence
  4-H3 VH
QVQLVESGGGVVQPGRSLRLSCAASGIDLTSNAMNWVRQAPGKGLEWIGGIYGHDTSYYAAWAKGRFTISRDNSKNTVYLQMNSLRAEDTATYFCARGFIYFDIWGQGTLVTVSS

SEQ ID NO: 172 - protein, 112 aa, Artificial Sequence
  4-H3 and 1-A2 VH
QQLVESGGGVVQPGRSLRLSCAASGIDLTSNAMNWVRQAPGKGLEWIGGIYGHDTSYYAAWAKGRFTISRDSNTVYLQMNSLRAEDTATYFCARGFIYFDIWGQGTLVTVSS

SEQ ID NO: 173 - protein, 8 aa, Artificial Sequence
  1-D5 CDR-H1
GFTFSNYI

SEQ ID NO: 174 - protein, 119 aa, Artificial Sequence
  1-D5 VH
QVQLVESGGGVVQPGRSLRLSCAASGFTFSNYIMGWVRQAPGKGLEWVAIISTGGSTYYASWAKGRFTISRDNSKNTLYLQMNSLRAEDTAVYYCARGGSSAGAGFNIWGQGTLVTVSS

SEQ ID NO: 175 - protein, 119 aa, Artificial Sequence
  1-D5 VH
QVQLVESGGGVVQPGRSLRLSCAASGFSLNNYIMGWVRQAPGKGLEWVAIISTGGSTYYASWAKGRFTISRDNSKNTLYLQMNSLRAEDTAVYYCARGGSSAGAGFNIWGQGTLVTVSS

SEQ ID NO: 176 - protein, 117 aa, Artificial Sequence
  1-D5 VH
QQQLVESGGGVVQPGRSLRLSCAASGFSLNNYIMGWVRQAPGKGLEYIGIISTGGSTYYASWAKGRFTISRDSNTMYLQMNSLRAEDTATYFCARGGSSAGAGFNIWGQGTLVTVSS

SEQ ID NO: 177 - protein, 8 aa, Artificial Sequence
  3-H9 CDR-H1
GFTFSNYD

SEQ ID NO: 178 - protein, 115 aa, Artificial Sequence
  3-H9 VH
QVQLVESGGGVVQPGRSLRLSCAASGFTFSNYDMSWVRQAPGKGLEWVASIHAIGITYYANWAEGRFTISRDNSKNTLYLQMNSLRAEDTAVYYCARGLVDLNMWGQGTLVTVSS

SEQ ID NO: 179 - protein, 115 aa, Artificial Sequence
  3-H9 VH
QVQLVESGGGVVQPGRSLRLSCAASGFSLSNYDMSWVRQAPGKGLEWVASIHAIGITYYANWAEGRFTISRDNSKNTLYLQMNSLRAEDTAVYYCARGLVDLNMWGQGTLVTVSS

SEQ ID NO: 180 - protein, 112 aa, Artificial Sequence
  3-H9 VH
QQLVESGGGVVQPGRSLRLSCAASGFSLSNYDMSWVRQAPGKGLEWIGSIHAIGITYYANWAEGRFTISRDKNTVYLQMNSLRAEDTATYFCARGLVDLNMWGQGTLVTVSS

SEQ ID NO: 181 - protein, 8 aa, Artificial Sequence
  1-G5 CDR-H1
GFTFSSYD

SEQ ID NO: 182 - protein, 115 aa, Artificial Sequence
  1-G5 VH
QVQLVESGGGVVQPGRSLRLSCAASGFTFSSYDMTWVRQAPGKGLEWVASIHATGITFYANWAKGRFTISRDNSKNTLYLQMNSLRAEDTAVYYCARGLVDLNMWGQGTLVTVSS

SEQ ID NO: 183 - protein, 115 aa, Artificial Sequence
  1-G5 VH
QVQLVESGGGVVQPGRSLRLSCAASGFSLSSYDMTWVRQAPGKGLEWVASIHATGITFYANWAKGRFTISRDNSKNTLYLQMNSLRAEDTAVYYCARGLVDLNMWGQGTLVTVSS

SEQ ID NO: 184 - protein, 112 aa, Artificial Sequence
  1-G5 VH
QQLVESGGGVVQPGRSLRLSCAASGFSLSSYDMTWVRQAPGKGLEWIGSIHATGITFYANWAKGRFTTSKDSNTVYLQMNSLRAEDTATYFCARGLVDLNMWGQGTLVTVSS

SEQ ID NO: 185 - protein, 110 aa, Artificial Sequence
  4-H3 VL
DIQMTQSPSSLSASVGDRVTITCLASEDIASGISWYQQKPGKAPKLLIYGASNLESGVPSRFSGSGSGTDFTLTISSLQPEDFATYYCLGGYSFSSNGLTFGGGTKVEIK

SEQ ID NO: 186 - protein, 110 aa, Artificial Sequence
  4-H3 VL
DYQMTQSPSSLSASVGDRVTITCLASEDIASGISWYQQKPGKAPKLLIYGASNLESGVPSRFSGSGSGTDYTLTISSLQPEDFATYYCLGGYSFSSNGLTFGGGTKVEIK

SEQ ID NO: 187 - protein, 110 aa, Artificial Sequence
  1-D5 VL
DIQMTQSPSSLSASVGDRVTITCQASQSINSGLAWYQQKPGKAPKLLIYKASTLASGVPSRFSGSGSGTDFTLTISSLQPEDFATYYCQSYHYISANGATFGGGTKVEIK

SEQ ID NO: 188 - protein, 110 aa, Artificial Sequence
  1-D5 VL
DIQMTQSPSSLSASVGDRVTITCQASQSINSGLAWYQQKPGKAPKRLIYKASTLASGVPSRFSGSGSGTDFTLTISSLQPEDFATYYCQSYHYISANGATFGGGTKVEIK

SEQ ID NO: 189 - protein, 112 aa, Artificial Sequence
  3-H9 VL
DIQMTQSPSSLSASVGDRVTITCQSSQSVYSNNLLSWYQQKPGKAPKLLIYDASTLESGVPSRFSGSGSGTDFTLTISSLQPEDFATYYCQGSYYSSGWYNTFGGGTKVEIK

SEQ ID NO: 190 - protein, 110 aa, Artificial Sequence
  1-A2 VL
DIQMTQSPSSLSASVGDRVTITCLASEDIYSGISWYQQKPGKAPKLLIYGASNLESGVPSRFSGSGSGTDFTLTISSLQPEDFATYYCLGGHSHSTTDLTFGGGTKVEIK

SEQ ID NO: 191 - protein, 110 aa, Artificial Sequence
  1-A2 VL
DIQMTQSPSSLSASVGDRVTITCLASEDIYSGISWYQQKPGKAPKLLIYGASNLESGVPSRFSGSGSGTDYTLTISSLQPEDFATYYCLGGHSHSTTDLTFGGGTKVEIK

SEQ ID NO: 192 - protein, 112 aa, Artificial Sequence
  1-G5 VL
DIQMTQSPSSLSASVGDRVTITCQASQSVYNNNYLSWYQQKPGKAPKLLIYDASTLASGVPSRFSGSGSGTDFTLTISSLQPEDFATYYCQGSYYSGGWDTAFGGGTKVEIK

SEQ ID NO: 193 - protein, 112 aa, Artificial Sequence
  1-G5 VL
DPQMTQSPSSLSASVGDRVTITCQASQSVYNNNYLSWYQQKPGKAPKLLIYDASTLASGVPSRFSGSGSGTDFTLTISSLQPEDFATYYCQGSYYSGGWDTAFGGGTKVEIK

SEQ ID NO: 194 - protein, 115 aa, Artificial Sequence
  4-H3 and 1-A2 VH
QVQLVESGGGVVQPGRSLRLSCAASGIDLTSNAMNWVRQAPGKGLEWVAGIYGHDTSYYAAWAKGRFTISRDNSKNTLYLQMNSLRAEDTAVYYCARGFIYFDIWGQGTLVTVSS

(19) United States
(12) Patent Application Publication
(10) Pub. No.: US 2024/0228660 A1
(43) Pub. Date: Jul. 11, 2024

(54) VON WILLEBRAND FACTOR (VWF) INHIBITORS

(71) Applicant: IP2IPO Innovations Ltd, London (GB)

(72) Inventors: Nadine Clemo, Cambridge (GB); Alan Lewis, Brentford (GB); Tom McKinnon, London (GB)

(21) Appl. No.: 18/556,638

(22) PCT Filed: Apr. 20, 2022
(86) PCT No.: PCT/GB2022/050989

§ 371 Date: Oct. 20, 2023

(30) Foreign Application Priority Data
Apr. 20, 2021 (GB) 2105625.4

(51) Int. Cl. C07K 16/36; C12N 15/113; G01N 33/68

(52) U.S. Cl. CPC C07K 16/36; C12N 15/113; G01N 33/6893; C12N 2310/11; C12N 2310/14; G01N 2333/755

(57) ABSTRACT

The invention relates to inhibitors of Von Willebrand Factor (VWF), and particularly to anti-VWF antibodies. The invention extends to compositions comprising the inhibitors, including pharmaceutical compositions and kits. The invention also extends to methods of making and using the inhibitors, for example in therapy and diagnosis of conditions caused by platelet-mediated aggregation, including various cardiovascular diseases, such as acquired thrombotic thrombocytopenia purpura (aTTP), ischemic stroke and atherosclerosis.

The present invention relates to inhibitors of Von Willebrand Factor (VWF), and particularly, although not exclusively, to inhibitors which target the C1-C6 domain of VWF. The invention extends to compositions comprising the inhibitors, including pharmaceutical compositions and kits. The invention also extends to methods of using the inhibitors, for example in therapy and diagnosis of conditions caused by platelet-mediated aggregation, including various cardiovascular diseases, such as acquired thrombotic thrombocytopeniarpura (aTTP), ischemic stroke and atherosclerosis.

Cardiovascular diseases (CVDs), including ischemic heart disease, stroke, heart failure, peripheral arterial disease, and a number of other cardiac and vascular conditions, remain the leading cause of death globally. CVDs are characterised by thrombotic events, caused by uncontrolled platelet aggregation, that contribute to both cell death and organ failure. Given the central role of platelets in triggering thrombosis, several approved antithrombotic drugs target platelets, such as aspirin, clopidogrel, and abciximab. Due to their complementary mechanisms of action, the combination of these agents inhibits platelet aggregation to a greater extent than any of the agents acting alone. However, the use of these antiplatelet drugs is hampered by an increased bleeding risk, reducing their application in wider patient populations. Therefore, there remains a high unmet medical need for therapies that can treat thrombotic disorders without the severe risk of bleeding.

Von Willebrand Factor (VWF), a large multimeric glycoprotein, present in blood plasma, endothelial cells, megakaryocytes, and platelets, is a well-established risk factor of thrombotic events. VWF plays a major role in haemostasis, mediating platelet adhesion to vascular injury sites, and protecting coagulation factor VIII (FVIII) from degradation. Following injury, collagen is exposed in the damaged vessel wall to flowing blood and shear forces. Plasma VWF binds to the exposed collagen and uncoils its structure, supporting the adhesion of circulating platelets. The bound VWF then interacts with the platelet receptor GPIb and platelet tethering occurs. Platelet plug formation is achieved once a critical mass of platelets, VWF and associated coagulation proteins bind together to seal the vessel wall. Other functions have also been reported for VWF, including regulation of inflammation and angiogenesis.

The accumulation of VWF has been associated with increased risk to CVDs. In particular, the accumulation of ultra-long VWF (ULVWF) in thrombotic thrombocytopenia purpura (TTP) patients, has been widely studied. Currently, Caplacizumab (a monoclonal antibody) is the only approved anti-VWF therapy, which has been shown to block the binding of VWF to platelets and reduce thrombi formation in TTP patients. However, this antibody functions by targeting and inhibiting the A1 domain of VWF (see FIG. 1). The A1 domain is essential for collagen binding, and therefore platelet binding, under low shear conditions, for normal haemostasis to take place. As such, by targeting this region, treatment with Caplacizumab results in a severe bleeding risk in patients. For patients suffering from TTP, a rare and fatal blood clotting disorder, the benefit of taking Caplacizumab outweighs the risk of severe bleeding. However, this severe bleeding risk is not acceptable for patients suffering from other CVDs, including ischemic stroke and myocardial infarction. Therefore, there exists a significant unmet medical need for new antithrombotic therapies that can be used to treat a wider range of thrombotic disorders, without the risk of severe bleeding.

The inventors have identified that a previously untargeted region of VWF, within the C1-C6 domain (see FIG. 1), is critical for the binding of VWF to collagen, allowing platelets to clot under high shear rates, such as those found in thrombotic conditions. Importantly, the C1-C6 domain is not essential for collagen binding, and therefore platelet binding, under low shear conditions (i.e. normal bleeding). Accordingly, this identifies the C1-C6 domain of VWF as a potential new therapeutic target for the treatment of a number of conditions caused by platelet-mediated aggregation or thrombotic-related conditions, including aTTP, ischemic stroke and atherosclerosis.

As such, the inventors hypothesised that by inhibiting the ability of the VWF C1-C6 region to bind to collagen under high shear rates, they could reduce platelet clotting in thrombotic conditions. Importantly, by targeting this region, VWF retains its ability to bind to platelets as normal under low shear rates (via the A1 domain), for normal haemostasis to occur, and so does not suffer from the significant problems associated with using Caplacizumab. This has led to the inventors' further work in developing antibodies that are capable of targeting the C1-C6 domain of VWF to inhibit its pro-thrombotic function. These anti-VWF monoclonal antibodies may be used in the treatment, amelioration or prevention of a number of thrombotic-related conditions, and would be much safer than the currently available treatments, as there would be a reduced risk of severe bleeding.

Accordingly, in a first aspect of the invention, there is provided an inhibitor that specifically binds to one or more of a C1, C2, C3, C4, C5, and/or C6 domain of Von Willebrand Factor (VWF).

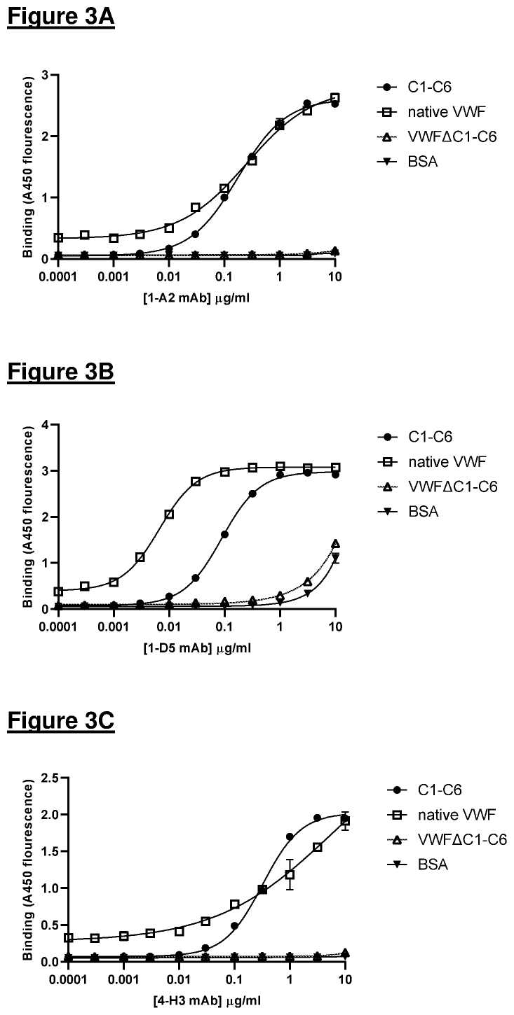

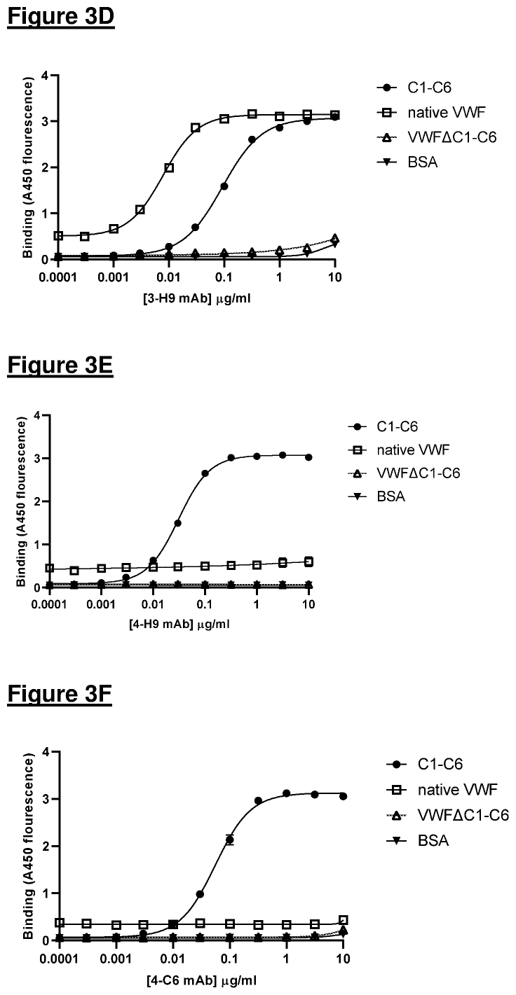

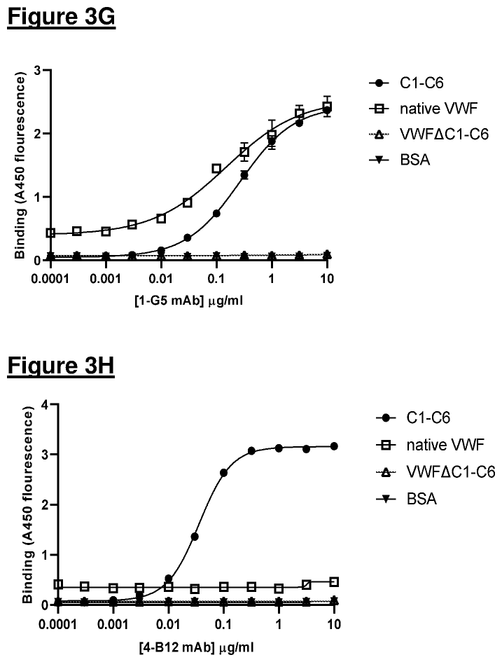

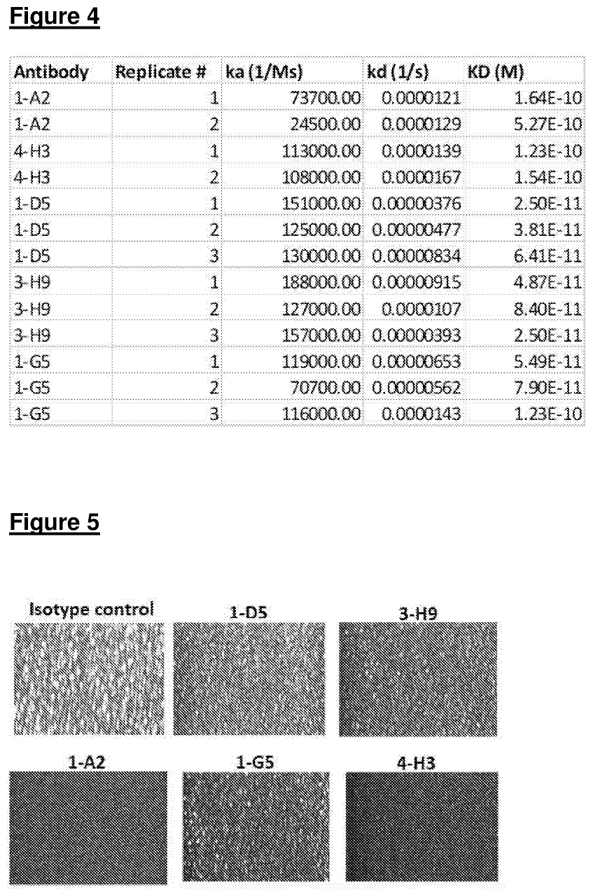

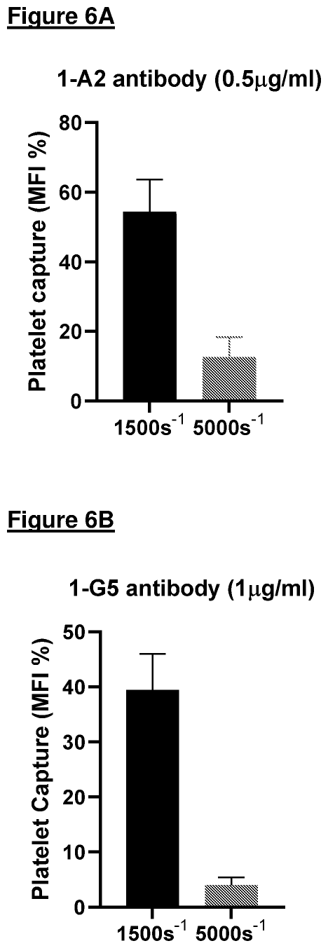

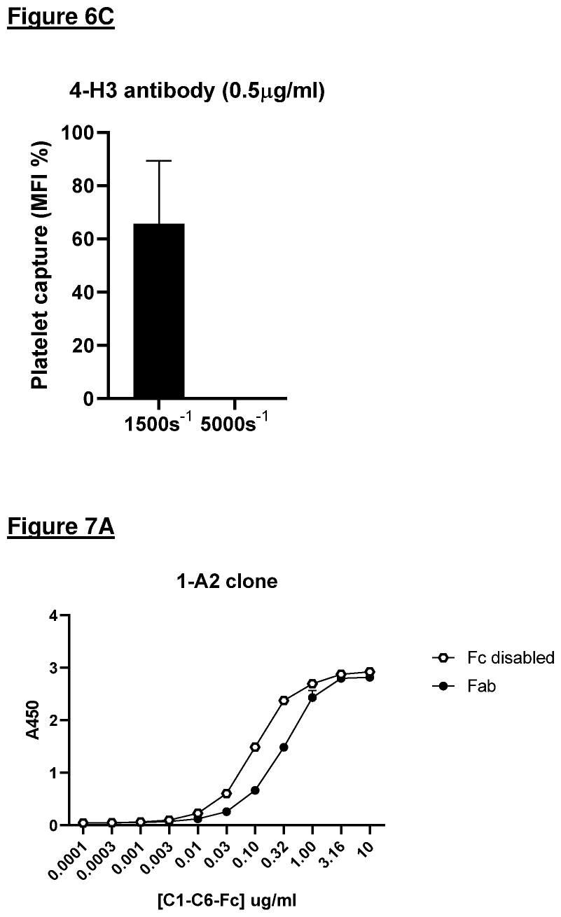

As shown in the examples, the inventors have developed antibodies that are capable of binding to, and inhibiting the function of the C1-C6 domains of VWF. For example, as shown in FIGS. 3A-3H, the inventors have developed eight antibodies that target within the C1-C6 domains of VWF. Furthermore, as shown in FIGS. 5 and 6, the inventors have demonstrated that surprisingly the antibodies significantly reduce platelet aggregation under high shear rates, i.e. pathological conditions, but under low shear rates, i.e. normal conditions, platelet capture is preserved. This demonstrates that targeting one or more of the C1-C6 domains of VWF, inhibits the pro-thrombotic function of VWF, without inhibiting its normal haemostatic function.

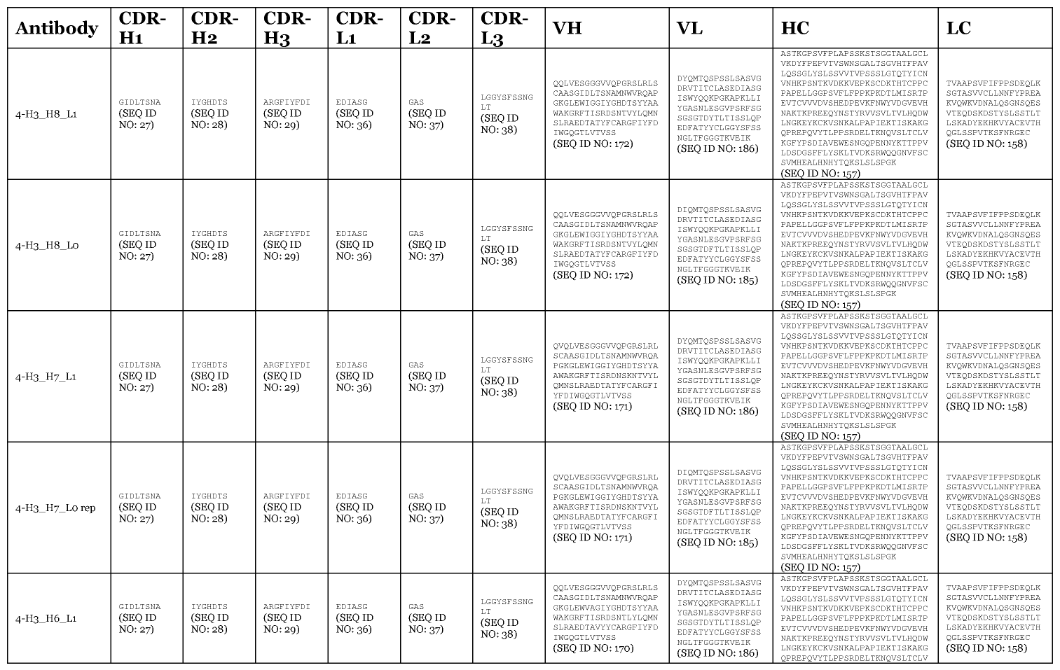

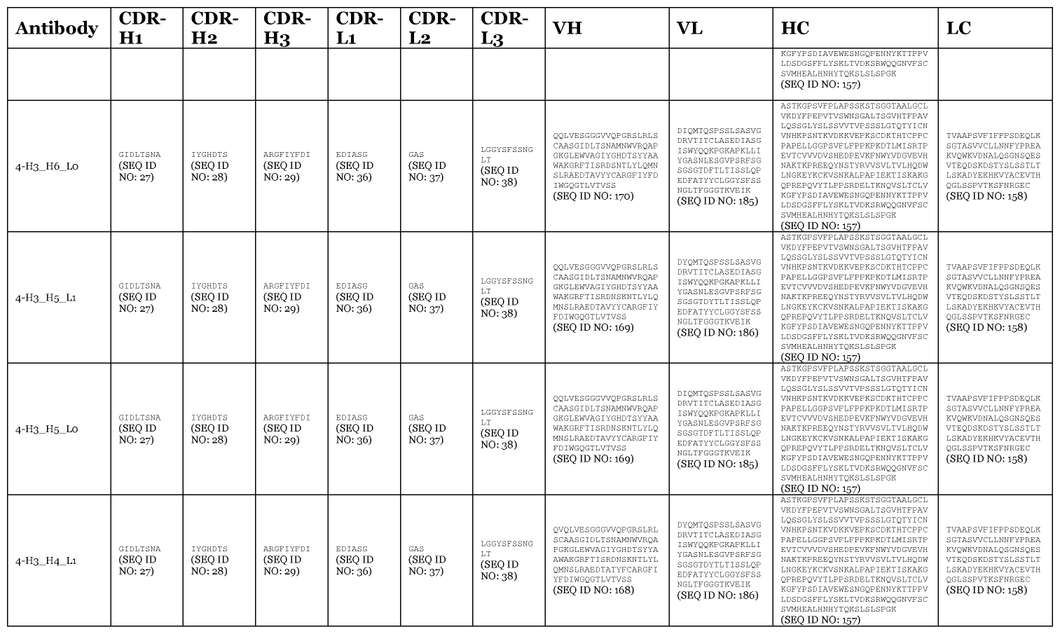

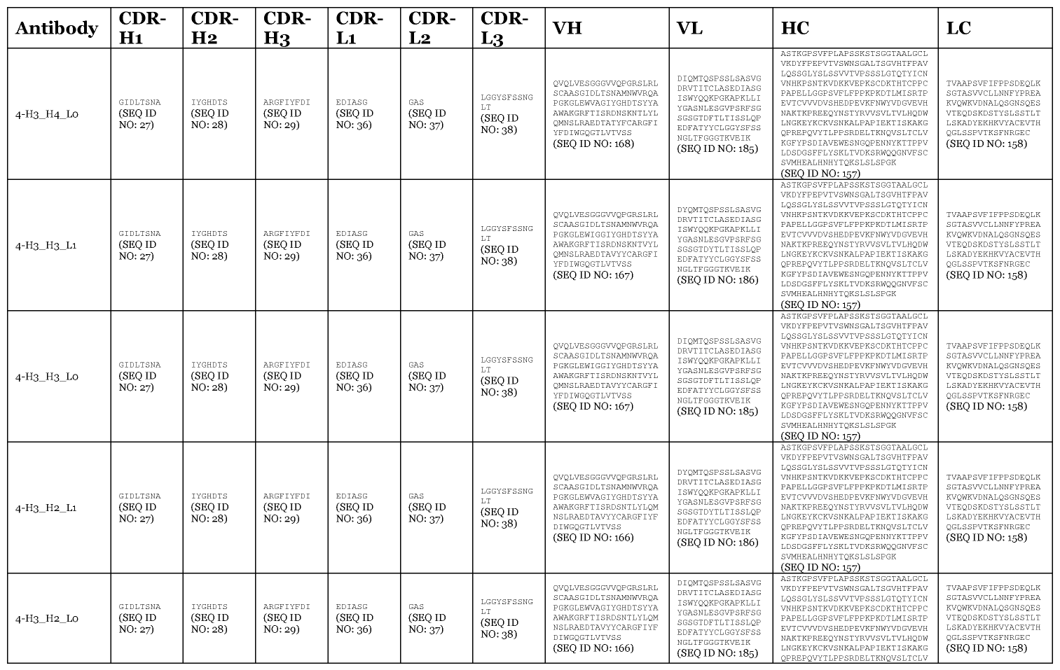

The inventors have demonstrated that monoclonal antibodies according to the invention bind to the C5 domain of VWF, with sub-nM affinities (see FIG. 9). Additionally, some of the antibody clones demonstrated weaker binding to the C3 (antibody 1-D5) and C4 (antibody 3-H9) domains of VWF.

Thus, in one preferred embodiment, the inhibitor of the invention specifically binds to the C3 and C5 domains of VWF (antibody 1-D5). Alternatively, in another preferred embodiment, the inhibitor of the invention specifically binds to the C4 and C5 domains of VWF (antibody 3-H9).

In another preferred embodiment, the inhibitor of the invention specifically binds to the C5 domain of VWF. In this embodiment, the inhibitor may additionally bind to one or more of the C1, C2, C3, C4 and/or C6 domains. However, the inhibitor of the invention may not substantially bind to the C1, C2, C3, C4 and/or C6 domains of VWF. Preferably, the inhibitor of the invention has substantially no cross-reactivity with the C1, C2, C3, C4 and/or C6 domains of VWF. Preferably, the inhibitor does not substantially bind to the C3 and/or C4 domains of VWF.

Preferably, the inhibitor of the invention is capable of inhibiting the function of one or more of a C1, C2, C3, C4, C5, and/or C6 domain of VWF. In a preferred embodiment, the inhibitor of the invention is capable of inhibiting the function of the C5 domain of VWF. Preferably, the inhibitor is capable of inhibiting the function of one or more of a C1, C2, C3, C4, C5, and/or C6 domain of VWF, such that platelet binding is inhibited under conditions of high shear rate, i.e. pathological conditions. Preferably, the inhibitor is capable of inhibiting the function of one or more of a C1, C2, C3, C4, C5, and/or C6 domain of VWF, such that platelet binding is not inhibited under conditions of low shear rate, i.e. normal conditions.

In one embodiment, the amino acid sequence of VWF may be represented by Genbank ID No: NM_000552.5, which is provided herein as SEQ ID No: 1, as follows:

[SEQ ID No: 1]
MIPARFAGVLLALALILPGTLCAEGTRGRSSTARCSLFGSDFVNTFDGSMYSFAGYCSYLLAGGCQKRSFSIIGDFQN
GKRVSLSVYLGEFFDIHLFVNGTVTQGDQRVSMPYASKGLYLETEAGYYKLSGEAYGFVARIDGSGNFQVLLSDRYEN
KTCGLCGNENIFAEDDEMTQEGTLTSDPYDFANSWALSSGEQWCERASPPSSSCNISSGEMQKGLWEQCQLLKSTSVE
ARCHPLVDPEPFVALCEKTLCECAGGLECACPALLEYARICAQEGMVLYGWTDHSACSPVCPAGMEYRQCVSPCARTC
QSLHINEMCQERCVDGCSCPEGQLLDEGLCVESTECPCVHSGKRYPPGTSLSRDCNTCICRNSQWICSNEECPGECLV
TGQSHFKSFDNRYFTFSGICQYLLARDCQDHSFSIVIETVQCADDRDAVCTRSVTVRLPGLHNSLVKLKHGAGVAMDG
QDVQLPLLKGDLRIQHTVTASVRLSYGEDLQMDWDGRGRLLVKLSPVYAGKTCGLCGNYNGNQGDDFLIPSGLAEPRV
EDFGNAWKLHGDCQDLQKQHSDPCALNPRMTRFSEEACAVLTSPTFEACHRAVSPLPYLRNCRYDVCSCSDGRECLCG
ALASYAAACAGRGVRVAWREPGRCELNCPKGQVYLQCGTPCNLTCRSLSYPDEECNEACLEGCFCPPGLYMDERGDCV
PKAQCPCYYDGEIFQPEDIFSDHHTMCYCEDGFMHCTMSGVPGSLLPDAVLSSPLSHRSKRSLSCRPPMVKLVCPADN
LRAEGLECTKTCQNYDLECMSMGCVSGCLCPPGMVRHENRCVALERCPCFHQGKEYAPGETVKIGCNTCVCQDRKWNC
TDHVCDATCSTIGMAHYLTFDGLKYLEPGECQYVLVQDYCGSNPGTFRILVGNKGCSHPSVKCKKRVTILVEGGEIEL
FDGEVNVKRPMKDETHFEVVESGRYIILLLGKALSVVWDRHLSISVVLKQTYQEKVCGLCGNEDGIQNNDLISSNLQV
EEDPVDFGNSWKVSSQCADTRKVPLDSSPATCHNNIMKQTMVDSSCRILTSDVFQDCNKLVDPEPYLDVCIYDTCSCE
SIGDCACFCDTIAAYAHVCAQHGKVVTWRTATLCPQSCEERNLRENGYECEWRYNSCAPACQVTCQHPEPLACPVQCV
EGCHAHCPPGKILDELLQTCVDPEDCPVCEVAGRRFASGKKVTLNPSDPEHCQICHCDVVNLTCEACQEPGGLVVPPT
DAPVSPTTLYVEDISEPPLHDFYCSRLLDLVFLLDGSSRLSEAEFEVLKAFVVDMMERLRISQKWVRVAVVEYHDGSH
AYIGLKDRKRPSELRRIASQVKYAGSQVASTSEVLKYTLFQIFSKIDRPEASRITLLLMASQEPQRMSRNFVRYVQGL
KKKKVIVIPVGIGPHANLKQIRLIEKQAPENKAFVLSSVDELEQQRDEIVSYLCDLAPEAPPPILPPDMAQVTVGPGL
LGVSTLGPKRNSMVLDVAFVLEGSDKIGEADFNRSKEFMEEVIQRMDVGQDSIHVTVLQYSYMVIVEYPFSEAQSKGD
ILQRVREIRYQGGNRINTGLALRYLSDHSFLVSQGDREQAPNLVYMVIGNPASDEIKRLPGDIQVVPIGVGPNANVQE
LERIGWPNAPILIQDFETLPREAPDLVLQRCCSGEGLQIPTLSPAPDCSQPLDVILLLDGSSSFPASYFDEMKSFAKA
FISKANIGPRLTQVSVLQYGSITTIDVPWNVVPEKAHLLSLVDVMQREGGPSQIGDALGFAVRYLTSEMHGARPGASK
AVVILVTDVSVDSVDAAADAARSNRVTVFPIGIGDRYDAAQLRILAGPAGDSNVVKLQRIEDLPTMVTLGNSFLHKLC
SGFVRICMDEDGNEKRPGDVWTLPDQCHTVTCQPDGQTLLKSHRVNCDRGLRPSCPNSQSPVKVEETCGCRWTCPCVC
TGSSTRHIVTFDGQNFKLTGSCSYVLFQNKEQDLEVILHNGACSPGARQGCMKSIEVKHSALSVELHSDMEVTVNGRL
VSVPYVGGNMEVNVYGAIMHEVRENHLGHIFTFTPQNNEFQLQLSPKTFASKTYGLCGICDENGANDEMLRDGTVTTD
WKTLVQEWTVQRPGQTCQPILEEQCLVPDSSHCQVLLLPLFAECHKVLAPATFYAICQQDSCHQEQVCEVIASYAHLC
RTNGVCVDWRTPDFCAMSCPPSLVYNHCEHGCPRHCDGNVSSCGDHPSEGCFCPPDKVMLEGSCVPEEACTQCIGEDG
VQHQFLEAWVPDHQPCQICTCLSGRKVNCTTQPCPTAKAPTCGLCEVARLRQNADQCCPEYECVCDPVSCDLPPVPHC
ERGLQPTLINPGECRPNFTCACRKEECKRVSPPSCPPARLPTLRKTQCCDEYECACNCVNSTVSCPLGYLASTATNDC
GCTTTTCLPDKVCVARSTIYPVGQFWEEGCDVCTCTDMEDAVMGLRVAQCSQKPCEDSCRSGFTYVLHEGECCGRCLP
SACEVVTGSPRGDSQSSWKSVGSQWASPENPCLINECVRVKEEVFIQQRNVSCPQLEVPVCPSGFQLSCKTSACCPSC
RCERMEACMLNGTVIGPGKTVMIDVCTTCRCMVQVGVISGFKLECRKTTCNPCPLGYKEENNTGECCGRCLPTACTIQ
LRGGQIMTLKRDETLQDGCDTHFCKVNERGEYFWEKRVTGCPPFDEHKCLAEGGKIMKIPGTCCDTCEEPECNDITAR
LQYVKVGSCKSEVEVDIRYCQGKCASKAMYSIDINDVQDQCSCCSPTRTEPMQVALHCINGSVVYREVLNAMECKCSP
RKCSK

The inhibitor may therefore bind to a region between amino acid positions 2255 and 2722 of SEQ ID No: 1, which correspond to C1-C6 domains of VWF.

Thus, preferably, the inhibitor may bind to one or more amino acids between amino acid positions 2255 and 2722 of VWF, corresponding to the C1-C6 domains, which is provided herein as SEQ ID No: 2, as follows:

[SEQ ID No: 2]
TQCIGEDGVQHQFLEAWVPDHQPCQICTCLSGRKVNCTTQPCPTAKAPTCGLCEVARLRQNADQCCPEYECVCDPVSC
DLPPVPHCERGLQPTLTNPGECRPNFTCACRKEECKRVSPPSCPPHRLPTLRKTQCCDEYECACNCVNSTVSCPLGYL
ASTATNDCGCTTTTCLPDKVCVHRSTIYPVGQFWEEGCDVCTCTDMEDAVMGLRVAQCSQKPCEDSCRSGFTYVLHEG
ECCGRCLPSACEVVTGSPRGDSQSSWKSVGSQWASPENPCLINECVRVKEEVFIQQRNVSCPQLEVPVCPSGFQLSCK
TSACCPSCRCERMEACMLNGTVIGPGKTVMIDVCTTCRCMVQVGVISGFKLECRKTTCNPCPLGYKEENNTGECCGRC
LPTACTIQLRGGQIMTLKRDETLQDGCDTHFCKVNERGEYFWEKRVTGCPPFDEHKCLAEGGKIMKIPGTCCDTCEEP

Thus, preferably the inhibitor binds to one or more amino acids within a sequence comprising or consisting of a sequence as substantially set out in SEQ ID No: 2, or a variant or fragment thereof.

In an embodiment, amino acid sequence of the C1 domain of VWF may be provided herein as SEQ ID No: 3, as follows:

[SEQ ID No: 3]
TQCIGEDGVQHQFLEAWVPDHQPCQICTCLSGRKVNCTTQPCPTAKAPT
CGLCEVARLRQNADQCCPEYECVCDPVSCD

Thus, preferably the inhibitor binds to one or more amino acids or an epitope within a sequence comprising or consisting of a sequence as substantially set out in SEQ ID No: 3, or a variant or fragment thereof.

In an embodiment, amino acid sequence of the C2 domain of VWF may be provided herein as SEQ ID No: 4, as follows:

[SEQ ID No: 4]
LPPVPHCERGLQPTLTNPGECRPNFTCACRKEECKRVSPPSCPPHRLPT
LRKTQCCDEYECACNCVNST

Thus, preferably the inhibitor binds to one or more amino acids or an epitope within a sequence comprising or consisting of a sequence as substantially set out in SEQ ID No: 4, or a variant or fragment thereof.

In an embodiment, amino acid sequence of the C3 domain of VWF may be provided herein as SEQ ID No: 5, as follows:

[SEQ ID No: 5]
VCVHRSTIYPVGQFWEEGCDVCTCTDMEDAVMGLRVAQCSQKPCEDSCR
SGFTYVLHEGECCGRCLP

Thus, preferably the inhibitor binds to one or more amino acids or an epitope within a sequence comprising or consisting of a sequence as substantially set out in SEQ ID No: 5, or a variant or fragment thereof.

In an embodiment, amino acid sequence of the C4 domain of VWF may be provided herein as SEQ ID No: 6, as follows:

[SEQ ID No: 6]
SACEVVTGSPRGDSQSSWKSVGSQWASPENPCLINECVRVKEEVFIQQR
NVSCPQLEVPVCPSGFQLSCKTSACCPSCRCE

Thus, preferably the inhibitor binds to one or more amino acids or an epitope within a sequence comprising or consisting of a sequence as substantially set out in SEQ ID No: 6, or a variant or fragment thereof.

In an embodiment, amino acid sequence of the C5 domain of VWF may be provided herein as SEQ ID No: 7, as follows:

[SEQ ID No: 7]
RMEACMLNGTVIGPGKTVMIDVCTTCRCMVQVGVISGFKLECRKTTCNP
CPLGYKEENNTGECCGRCLP

Thus, preferably the inhibitor binds to one or more amino acids or an epitope within a sequence comprising or consisting of a sequence as substantially set out in SEQ ID No: 7, or a variant or fragment thereof.

In an embodiment, amino acid sequence of the C6 domain of VWF may be provided herein as SEQ ID No: 8, as follows:

[SEQ ID No: 8]
TACTIQLRGGQIMTLKRDETLQDGCDTHFCKVNERGEYFWEKRVTGCPP
FDEHKCLAEGGKIMKIPGTCCDTCEEP

Thus, preferably the inhibitor binds to one or more amino acids or an epitope within a sequence comprising or consisting of a sequence as substantially set out in SEQ ID No: 8, or a variant or fragment thereof.

Previous therapeutic targeting of VWF has focused on the A1 and A3 domains, for example Caplacizumab which target A1, and 82D6A3 which targets A3. However, the A1 and A3 domains are essential for collagen binding and, therefore, platelet binding. As such, targeting of the A1 and A3 domains, inhibits platelet binding under conditions of low shear rate, i.e. normal conditions, resulting in a severe bleeding risk in patients.

Therefore, it is important that the inhibitor of the invention, which targets one or more of the C1, C2, C3, C4, C5, and/or C6 domains of VWF does so specifically, and has no or little cross-reactivity with the A1, A2, and/or A3 domains of VWF, because this could result in significant unwanted off-target effects, such as a severe risk of bleeding.

Accordingly, preferably the inhibitor of the invention does not substantially bind to an A1, A2, and/or A3 domain of VWF. Preferably, the inhibitor of the invention has substantially no cross-reactivity with a A1, A2, and/or A3 domain of VWF. Most preferably, the inhibitor of the invention has substantially no cross-reactivity with the A1 domain of VWF.

In an embodiment, amino acid sequence of the A1 domain of VWF may be provided herein as SEQ ID No: 153, as follows:

[SEQ ID No: 153]
DLVFLLDGSSRLSEAEFEVLKAFVVDMMERLRISQKWVRVAVVEYHDGS
HAYIGLKDRKRPSELRRIASQVKYAGSQVASTSEVLKYTLFQIFSKIDR
PEASRITLLLMASQEPQRMSRNFVRYVQGLKKKKVIVIPVGIGPHANLK
QIRLIEKQAPENKAFVLSSVDELEQQRDEI

Thus, preferably the inhibitor does not bind to a sequence as substantially set out in SEQ ID No: 153, or a variant or fragment thereof.

In an embodiment, amino acid sequence of the A2 domain of VWF may be provided herein as SEQ ID No: 154, as follows:

[SEQ ID No: 154]
DVAFVLEGSDKIGEADFNRSKEFMEEVIQRMDVGQDSIHVTVLQYSYMV
TVEYPFSEAQSKGDILQRVREIRYQGGNRTNTGLALRYLSDHSFLVSQG
DREQAPNLVYMVTGNPASDEIKRLPGDIQVVPIGVGPNANVQELERIGW
PNAPILIQDFETLPREAPDLVQRCC

Thus, preferably the inhibitor does not bind to a sequence as substantially set out in SEQ ID No: 154, or a variant or fragment thereof.

In an embodiment, amino acid sequence of the A3 domain of VWF may be provided herein as SEQ ID No: 155, as follows:

[SEQ ID No: 155]
DVILLLDGSSSFPASYFDEMKSFAKAFISKANIGPRLTQVSVLQYGSIT
TIDVPWNVVPEKAHLLSLVDVMQREGGPSQIGDALGFAVRYLTSEMHGA
RPGASKAVVILVTDVSVDSVDAAADAARSNRVTVFPIGIGDRYDAAQLR
ILAGPAGDSNVVKLQRIEDLPTMVTLGNSFLHKL

Thus, preferably the inhibitor does not bind to a sequence as substantially set out in SEQ ID No: 155, or a variant or fragment thereof.

In one preferred embodiment, the inhibitor is an antibody, or an antigen-binding fragment thereof.

Hence, in a further aspect of the invention, there is provided an antibody, or an antigen-binding fragment thereof inhibitor that specifically binds to one or more of a C1, C2, C3, C4, C5, and/or C6 domain of Von Willebrand Factor (VWF), and which preferably does not substantially bind to an A1, A2 and/or A3 domain of VWF.

The invention extends to both whole antibodies (i.e. immunoglobulins) with immunospecificity for one or more of the C1, C2, C3, C4, C5, and/or C6 domain of VWF (preferably C5), as well as to antigen-binding fragments or regions of the corresponding full-length antibody.

The antibody or antigen-binding fragment thereof may be monovalent, divalent or polyvalent. Monovalent antibodies are dimers (HL) comprising a heavy (H) chain associated by a disulphide bridge with a light chain (L). Divalent antibodies are tetramer (H2L2) comprising two dimers associated by at least one disulphide bridge. Polyvalent antibodies may also be produced, for example by linking multiple dimers.

The basic structure of an antibody molecule consists of two identical light chains and two identical heavy chains which associate non-covalently and can be linked by disulphide bonds. Each heavy and light chain contains an amino-terminal variable region of about 110 amino acids, and constant sequences in the remainder of the chain. The variable region includes several hypervariable regions, or Complementarity Determining Regions (CDRs), that form the antigen-binding site of the antibody molecule and determine its specificity for the antigen, i.e. one or more of a C1, C2, C3, C4, C5, and/or C6 domain of VWF (preferably C5), or variant or fragment thereof (e.g. an epitope). On either side of the CDRs of the heavy and light chains is a framework region, a relatively conserved sequence of amino acids that anchors and orients the CDRs. Antibody fragments may include a bi-specific antibody (BsAb) or a chimeric antigen receptor (CAR).

The heavy chain constant region typically comprises three domains, C_{H1}, C_{H2}, and C_{H3}. Each light chain typically comprises a light chain variable region (VL) and a light chain constant region. The light chain constant region typically comprises one domain, abbreviated CL.

Each heavy chain and light chain generally comprise three CDRs and four FRs, arranged in the following order (from N-terminus to C-terminus): FR1-CDR1-FR2-CDR2-FR3-CDR3-FR4. The CDRs are involved in antigen binding and confer antigen specificity and binding affinity to the antibody. See Kabat et al., Sequences of Proteins of Immunological Interest 5th ed. (1991) Public Health Service, National Institutes of Health, Bethesda, MD, incorporated by reference in its entirety.

The heavy chain from any vertebrate species can be assigned to one of five different classes (or isotypes): IgA, IgD, IgE, IgG, and IgM. These classes are also designated a, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2. The IgG antibody class is preferred.

The light chain from any vertebrate species can be assigned to one of two types, called kappa and lambda, based on the sequence of the constant domain.

The constant region consists of one of five heavy chain sequences (μ, γ, ζ, α, or ε) and one of two light chain sequences (κ or λ). The heavy chain constant region sequences determine the isotype of the antibody and the effector functions of the molecule.

Preferably, the antibody or antigen-binding fragment thereof is isolated or purified.

In one preferred embodiment, the antibody or antigen-binding fragment thereof comprises a polyclonal antibody, or an antigen-binding fragment thereof. The antibody or antigen-binding fragment thereof may be generated in a rabbit, mouse or rat.

Preferably, the antibody or antigen-binding fragment thereof is obtained by immunising a host animal with a C1-C6-Fc protein, or a variant or fragment thereof, such as any one or more of C1, C2, C3, C4, C5, and/or C6 domain, and then collecting the antibody or antigen-binding fragment thereof. The host animal may be a rabbit.

In another preferred embodiment, the antibody or antigen-binding fragment thereof comprises a monoclonal antibody or an antigen-binding fragment thereof. The antibody or fragment thereof of may be mammalian. Preferably, the antibody of the invention is a human antibody. As used herein, the term “human antibody” can mean an antibody, such as a monoclonal antibody, which comprises substantially the same heavy and light chain CDR amino acid sequences as found in a particular human antibody exhibiting immunospecificity for one or more of C1, C2, C3, C4, C5, and/or C6 domains of VWF (preferably C5), or a variant or fragment thereof. An amino acid sequence, which is substantially the same as a heavy or light chain CDR, exhibits a considerable amount of sequence identity when compared to a reference sequence. Such identity is definitively known or recognisable as representing the amino acid sequence of the particular human antibody. Substantially the same heavy and light chain CDR amino acid sequence can have, for example, minor modifications or conservative substitutions of amino acids. Such a human antibody or fragment thereof maintains its function of selectively binding to at least one of the C1, C2, C3, C4, C5, and/or C6 domains of VWF (preferably C5), or a variant or fragment thereof.

The term “human monoclonal antibody” can include a monoclonal antibody with substantially or entirely human CDR amino acid sequences produced, for example by recombinant methods, such as production by a phage library, by lymphocytes or by hybridoma cells.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (by so-called “affinity maturation”), to humanize the antibody, to improve its production in cell culture, and/or to reduce its immunogenicity in a subject.

The term “humanised antibody” can mean an antibody from a non-human species (e.g. mouse or rabbit) whose protein sequences have been modified to increase their similarity to antibodies produced naturally in humans.

The antibody may be a recombinant antibody. The term “recombinant human antibody” can include a human antibody produced using recombinant DNA technology.

The term “antigen-binding fragment” can mean a region of the antibody having specific binding affinity for its target antigen, for example, one or more of a C1, C2, C3, C4, C5, and/or C6 domain of VWF (preferably C5), or a variant or fragment thereof. Preferably, the fragment is an epitope. The epitope may be linear or conformational. The antigen-binding region may be a hypervariable CDR or a functional portion thereof. The term “functional portion” of a CDR can mean a sequence within the CDR which shows specific affinity for the target antigen. The functional portion of a CDR may comprise a ligand which specifically binds to one or more of a C1, C2, C3, C4, C5, and/or C6 domain of VWF (preferably C5), or a fragment thereof.

The term “CDR” can mean a hypervariable region in the heavy and light variable chains. There may be one, two, three or more CDRs in each of the heavy and light chains of the antibody. Normally, there are at least three CDRs on each chain which, when configured together, form the antigen-binding site, i.e. the three-dimensional combining site with which the antigen binds or specifically reacts. It has however been postulated that there may be four CDRs in the heavy chains of some antibodies.

The definition of CDR also includes overlapping or subsets of amino acid residues when compared against each other. The exact residue numbers which encompass a particular CDR or a functional portion thereof will vary depending on the sequence and size of the CDR. Those skilled in the art can routinely determine which residues comprise a particular CDR given the variable region amino acid sequence of the antibody.

The amino acid sequence boundaries of a CDR can be determined by using any of a number of known numbering schemes, including those described by Kabat et al., supra (“Kabat” numbering scheme); A1-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme).

The term “functional fragment” of an antibody can mean a portion of the antibody which retains a functional activity. A functional activity can be, for example antigen binding activity or specificity. A functional activity can also be, for example, an effector function provided by an antibody constant region. The term “functional fragment” is also intended to include, for example, fragments produced by protease digestion or reduction of a human monoclonal antibody and by recombinant DNA methods known to those skilled in the art. Human monoclonal antibody functional fragments include, for example individual heavy or light chains and fragments thereof, such as VL, VH and

Fd; monovalent fragments, such as Fv, Fab, and Fab′; bivalent fragments such as F(ab′)₂; single chain Fv (scFv); and Fc fragments. Alternatively, as discussed hereinafter, and as exemplified, the Fc fragment of the antibody may be disabled by introducing amino acid substitutions into the Fc region, which silence or reduce the effector function of the antibody.

The term “VL fragment” can mean a fragment of the light chain of a human monoclonal antibody which includes all or part of the light chain variable region, including the CDRs. A VL fragment can further include light chain constant region sequences.

The term “VH fragment” can mean a fragment of the heavy chain of a human monoclonal antibody which includes all or part of the heavy chain variable region, including the CDRs.

The term “Fd fragment” can mean the heavy chain variable region coupled to the first heavy chain constant region, i.e. VH and CH-1. The “Fd fragment” does not include the light chain, or the second and third constant regions of the heavy chain.

The term “Fv fragment” can mean a monovalent antigen-binding fragment of a human monoclonal antibody, including all or part of the variable regions of the heavy and light chains, and absent of the constant regions of the heavy and light chains. The variable regions of the heavy and light chains include, for example, the CDRs. For example, an Fv fragment includes all or part of the amino terminal variable region of about 110 amino acids of both the heavy and light chains.

The term “Fab fragment” can mean a monovalent antigen-binding fragment of a human monoclonal antibody that is larger than an Fv fragment. For example, a Fab fragment includes the variable regions, and all or part of the first constant domain of the heavy and light chains. Thus, a Fab fragment additionally includes, for example, amino acid residues from about 110 to about 220 of the heavy and light chains.

The term “Fab′ fragment” can mean a monovalent antigen-binding fragment of a human monoclonal antibody that is larger than a Fab fragment. For example, a Fab′ fragment includes all of the light chain, all of the variable region of the heavy chain, and all or part of the first and second constant domains of the heavy chain. For example, a Fab′ fragment can additionally include some or all of amino acid residues 220 to 330 of the heavy chain.

The term “F(ab′)₂ fragment” can mean a bivalent antigen-binding fragment of a human monoclonal antibody. An F(ab′)₂ fragment includes, for example, all or part of the variable regions of two heavy chains—and two light chains, and can further include all or part of the first constant domains of two heavy chains and two light chains.

The term “single chain Fv (scFv)” can mean a fusion of the variable regions of the heavy (VH) and light chains (VL) connected with a short linker peptide.

The term “bispecific antibody (BsAb)” can mean a bispecific antibody comprising two scFv linked to each other by a shorter linked peptide.

One skilled in the art knows that the exact boundaries of a fragment of an antibody are not important, so long as the fragment maintains a functional activity. Using well-known recombinant methods, one skilled in the art can engineer a polynucleotide sequence to express a functional fragment with any endpoints desired for a particular application. A functional fragment of the antibody may comprise or consist of a fragment with substantially the same heavy and light chain variable regions as the human antibody.

Preferably, the antibody or antigen-binding fragment thereof, with respect to the first aspect of the invention, is immunospecific for an epitope within one or more of a C1, C2, C3, C4, C5, and/or C6 domain of VWF. In one preferred embodiment, the antibody or antigen-binding fragment thereof, is immunospecific for an epitope within the C3 and C5 domains of VWF (antibody 1-D5). Alternatively, in another preferred embodiment, the antibody or antigen-binding fragment thereof, is immunospecific for an epitope within the C4 and C5 domains of VWF (antibody 3-H9). Even more preferably, the antibody or antigen-binding fragment thereof, is immunospecific for an epitope within the C5 domain of VWF. The antigen-binding fragment thereof may comprise or consist of any of the fragments selected from a group consisting of VH, VL, Fd, Fv, Fab, Fab′, scFv, F (ab′)₂ and Fc fragment.

The antigen-binding fragment thereof may be a single domain antibody (sdAb), otherwise referred to as a nanobody, which the skilled person would understand is an antibody fragment consisting of a single monomeric variable antibody domain.

The antigen-binding fragment thereof may comprise or consist of any one of the antigen binding region sequences of the VL, any one of the antigen binding region sequences of the VH, or a combination of VL and VH antigen binding regions of a human antibody. The appropriate number and combination of VH and VL antigen binding region sequences may be determined by those skilled in the art depending on the desired affinity and specificity and the intended use of the antigen-binding fragment. Functional fragments or antigen-binding fragments of antibodies may be readily produced and isolated using methods well known to those skilled in the art. Such methods include, for example, proteolytic methods, recombinant methods and chemical synthesis. Proteolytic methods for the isolation of functional fragments 30 comprise using human antibodies as a starting material. Enzymes suitable for proteolysis of human immunoglobulins may include, for example, papain, and pepsin. The appropriate enzyme may be readily chosen by one skilled in the art, depending on, for example, whether monovalent or bivalent fragments are required. For example, papain cleavage results in two monovalent Fab′ fragments that bind antigen and an Fc 35 fragment. Pepsin cleavage, for example, results in a bivalent F (ab′) fragment. An F (ab′)₂ fragment of the invention may be further reduced using, for example, DTT or 2-mercaptoethanol to produce two monovalent Fab′ fragments.

Functional or antigen-binding fragments of antibodies produced by proteolysis may be purified by affinity and column chromatographic procedures. For example, undigested antibodies and Fc fragments may be removed by binding to protein A. Additionally, functional fragments may be purified by virtue of their charge and size, using, for example, ion exchange and gel filtration chromatography. Such methods are well known to those skilled in the art.

The antibody or antigen-binding fragment thereof may be produced by recombinant methodology. Preferably, one initially isolates a polynucleotide encoding desired regions of the antibody heavy and light chains. Such regions may include, for example, all or part of the variable region of the heavy and light chains. Preferably, such regions can particularly include the antigen binding regions of the heavy and light chains, preferably the antigen binding sites, most preferably the CDRs.

The polynucleotide encoding the antibody or antigen-binding fragment thereof according to the invention may be produced using methods known to those skilled in the art. The polynucleotide encoding the antibody or antigen-binding fragment thereof may be directly synthesized by methods of oligonucleotide synthesis known in the art. Alternatively, smaller fragments may be synthesized and joined to form a larger functional fragment using recombinant methods known in the art.

As used herein, the term “immunospecificity” can mean the binding region of the antibody or antigen-binding fragment thereof is capable of immunoreacting with one or more of a C1, C2, C3, C4, C5, and/or C6 domain of VWF (preferably C5), or a variant or fragment thereof, by specifically binding therewith. The antibody or antigen-binding fragment thereof can preferably selectively interact with an antigen (one or more of a C1, C2, C3, C4, C5, and/or C6 domain of VWF—preferably C5) with an affinity constant of approximately 10⁻⁵ to 10⁻¹³ M⁻¹, preferably 10⁻⁶ to 10⁻⁹ M⁻¹, even more preferably, 10⁻¹⁰ to 10⁻¹² M⁻¹.

The antibody or antigen-binding fragment thereof preferably does not substantially bind to A1, A2, and/or A3 domains of VWF, such that the affinity constant is approximately more than 10⁻¹⁰ M⁻¹, 10⁻⁹ M⁻¹, 10⁻⁸ M⁻¹, 10⁻⁷ M⁻¹, or 10⁻⁶ M⁻¹, preferably more than 10⁻⁵ M⁻¹, 10⁻⁴M⁻¹ or 10⁻³M⁻¹ and even more preferably 10⁻²M⁻¹M⁻¹ or 10⁻²M⁻¹ and most preferably 10⁺¹M⁻¹, 10⁺²M⁻¹ or 10⁺³M⁻¹.

The term “immunoreact” can mean the binding region is capable of eliciting an immune response upon binding with one or more of a C1, C2, C3, C4, C5, and/or C6 domain of VWF, or an epitope thereof.

The term “epitope” can mean any region of an antigen with the ability to elicit, and combine with, a binding region of the antibody or antigen-binding fragment thereof.

The epitope may be linear. This can mean that the antibody interacts with a plurality of continuous amino acids of the antigen, and so the epitope can consist of these defined amino acids.

Alternatively, the epitope may be conformational, i.e. non-linear or discontinuous. This can mean that the antibody interacts with multiple, distinct segments from the primary amino acid sequence of the antigen.

Thus, the antibody or antigen-binding fragment thereof may comprise a heavy chain. The heavy chain may be selected from the group consisting of IgA; IgD; IgE; IgG and IgM. Preferably, the heavy chain is an IgG. Preferably, the heavy chain is an IgA.

The heavy chain may be an IgG1. The heavy chain may be an IgG2. The heavy chain may be an IgG3. The heavy chain may be an IgG4. The heavy chain may be an IgA1. The heavy chain may be an IgA2.

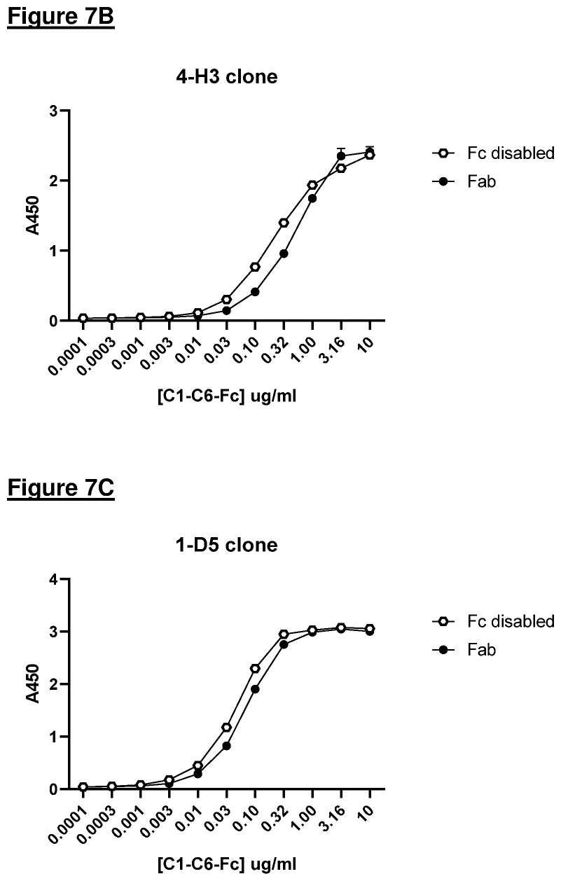

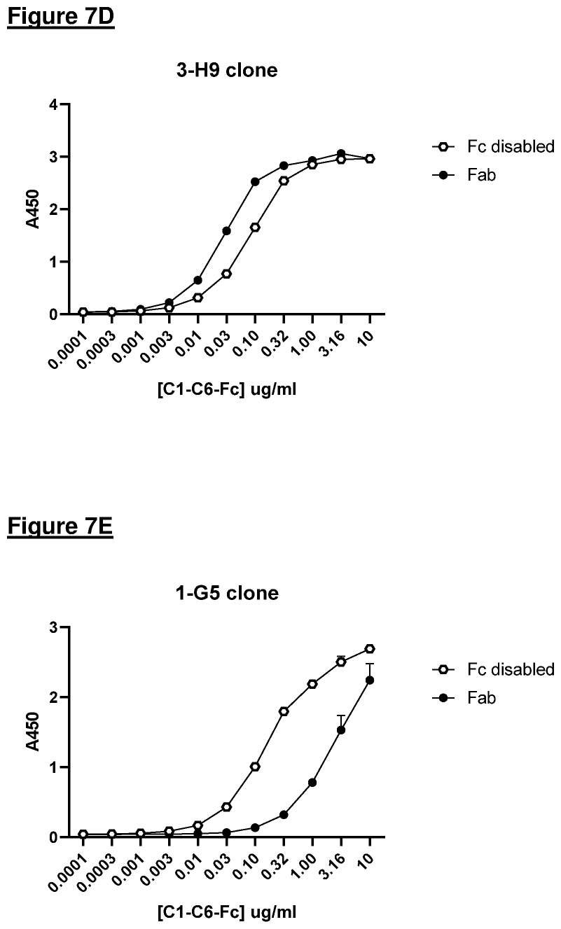

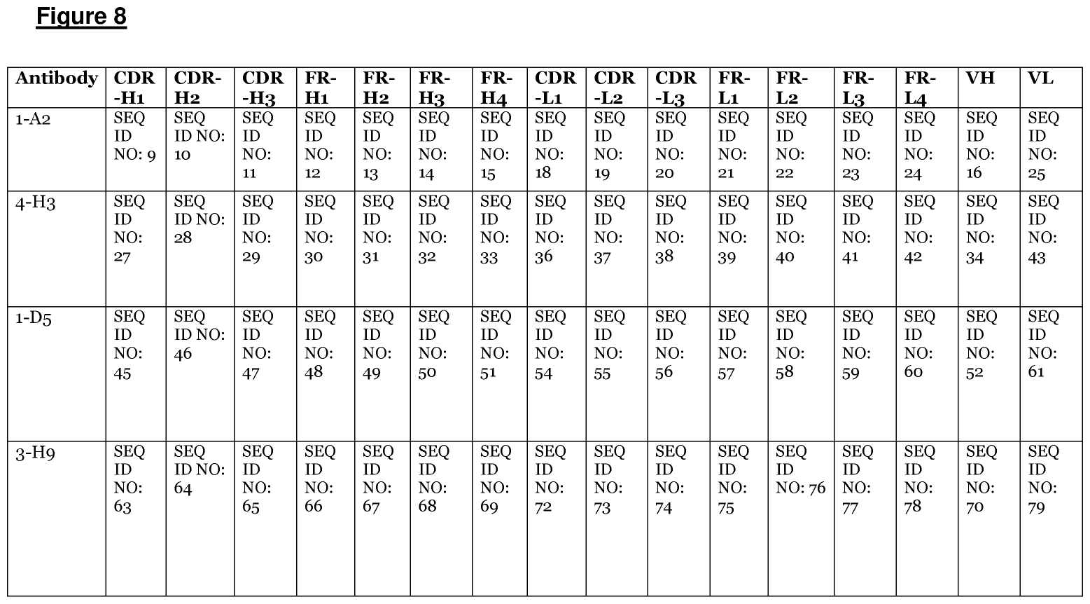

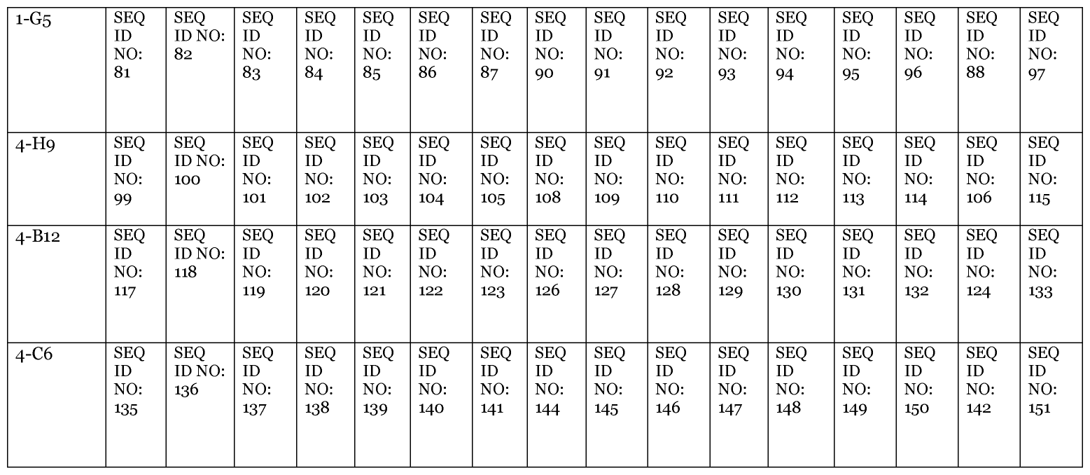

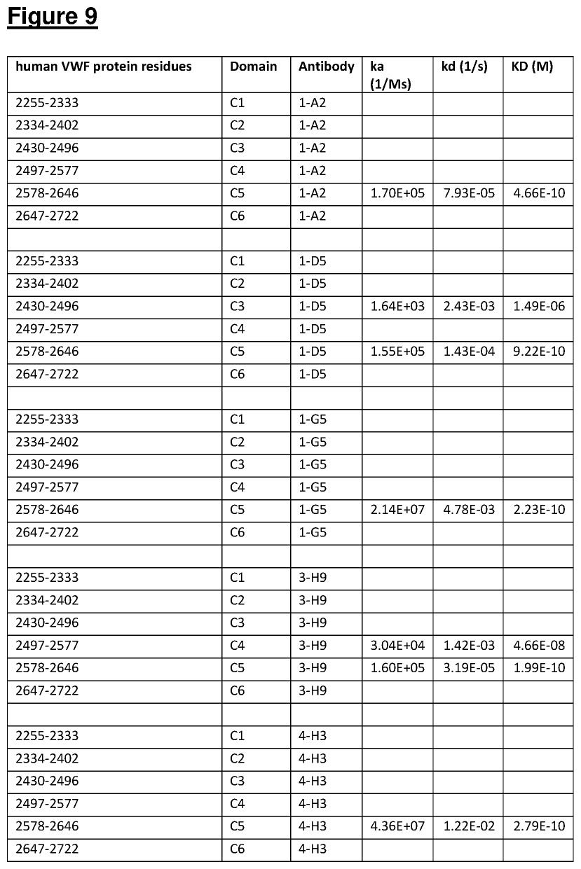

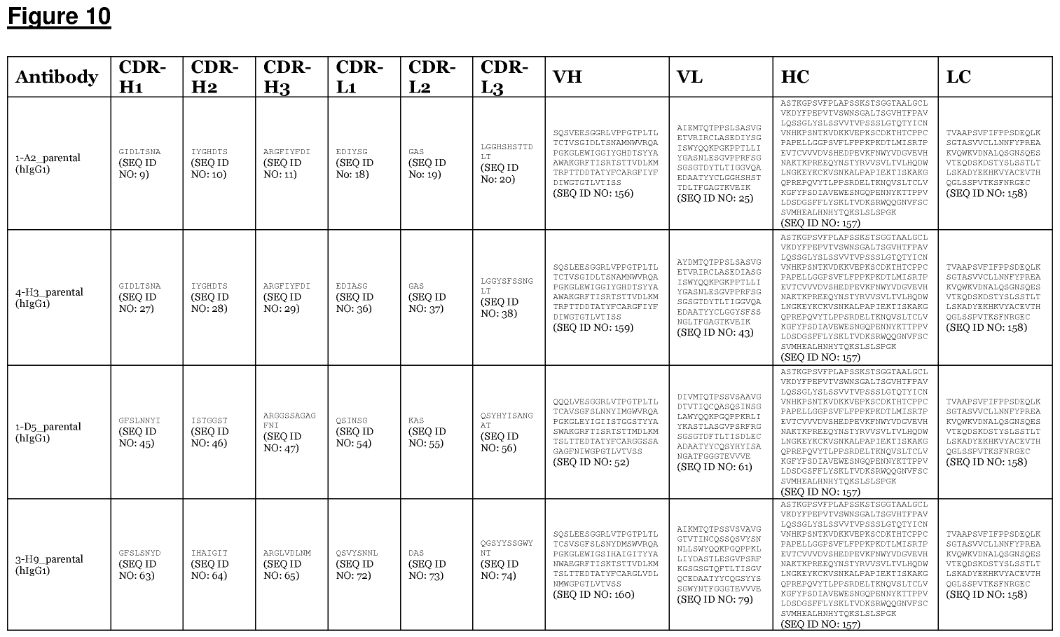

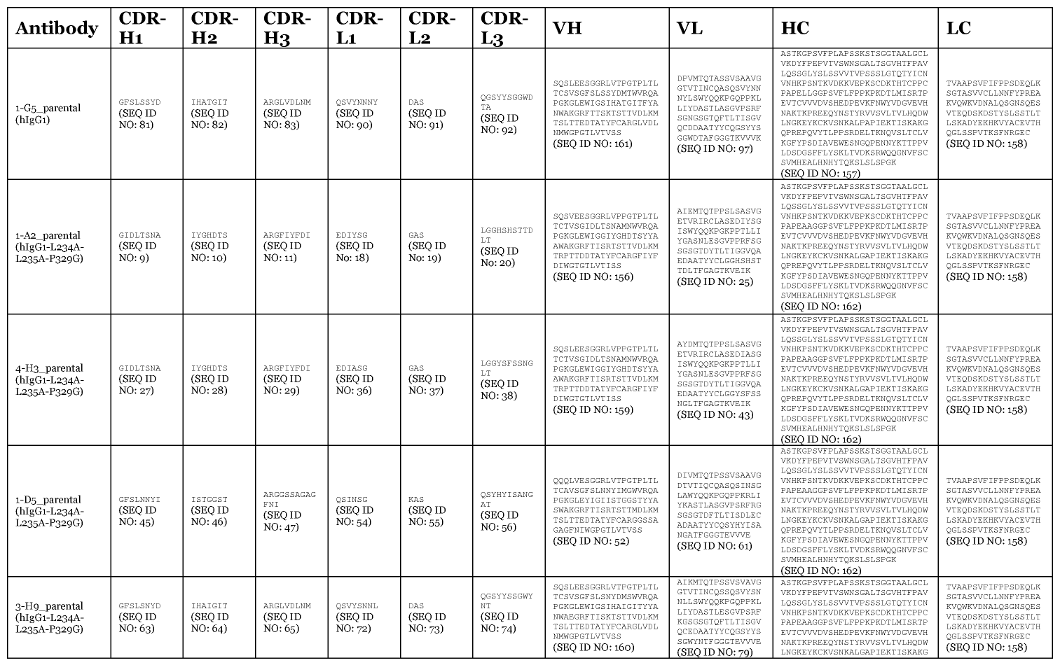

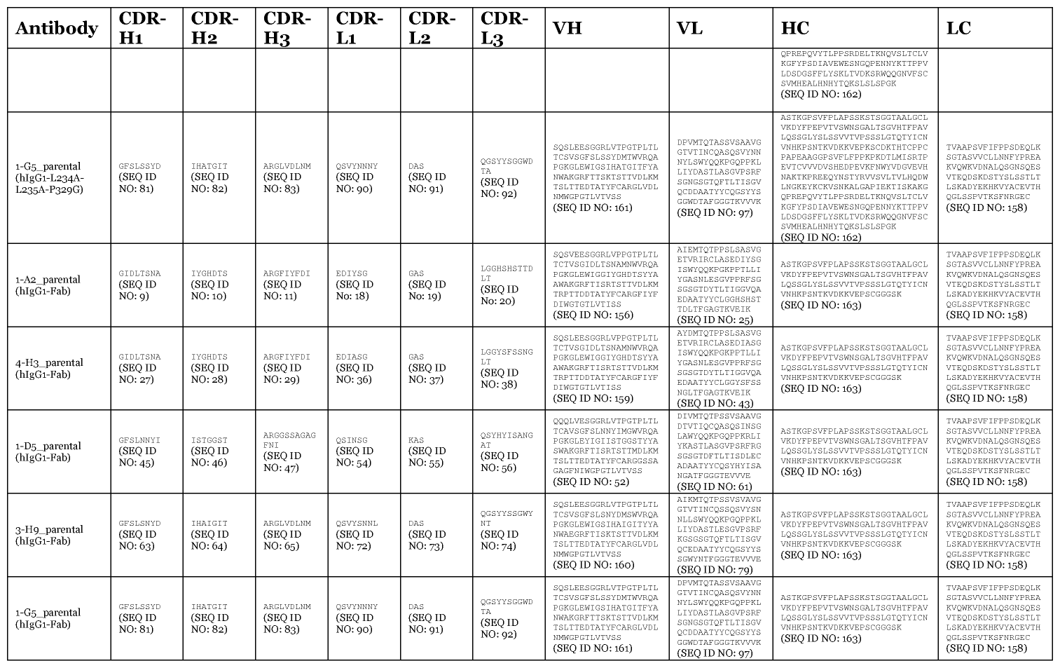

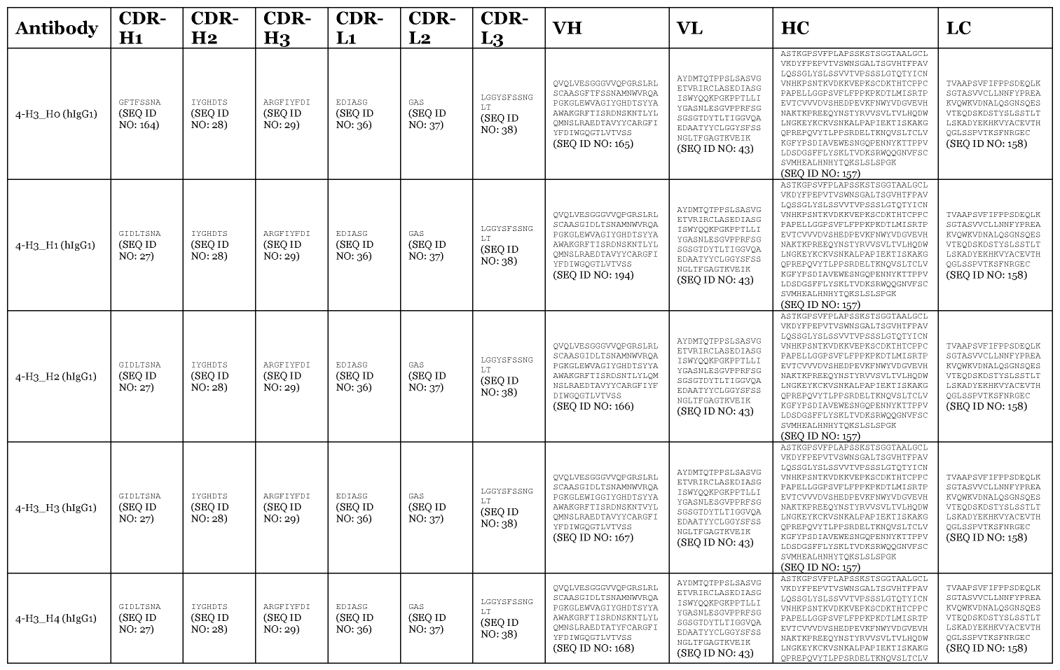

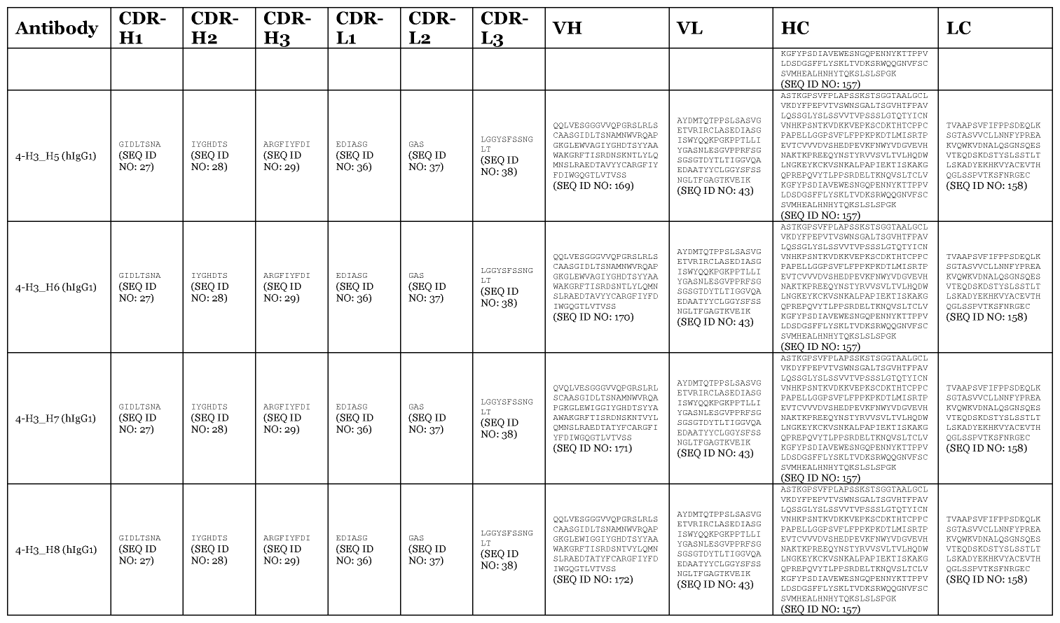

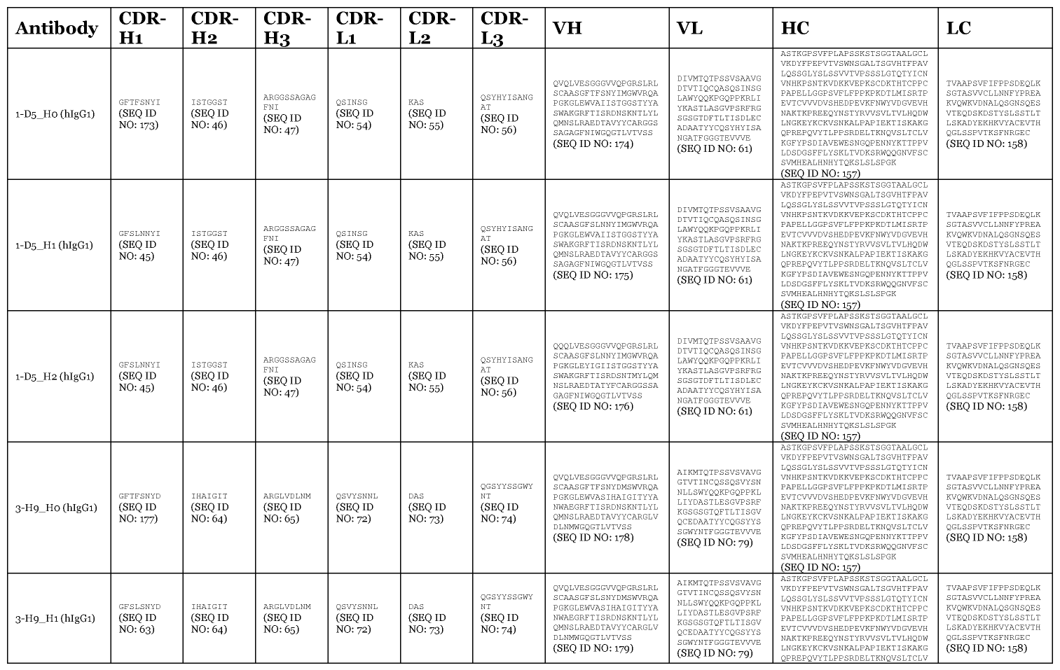

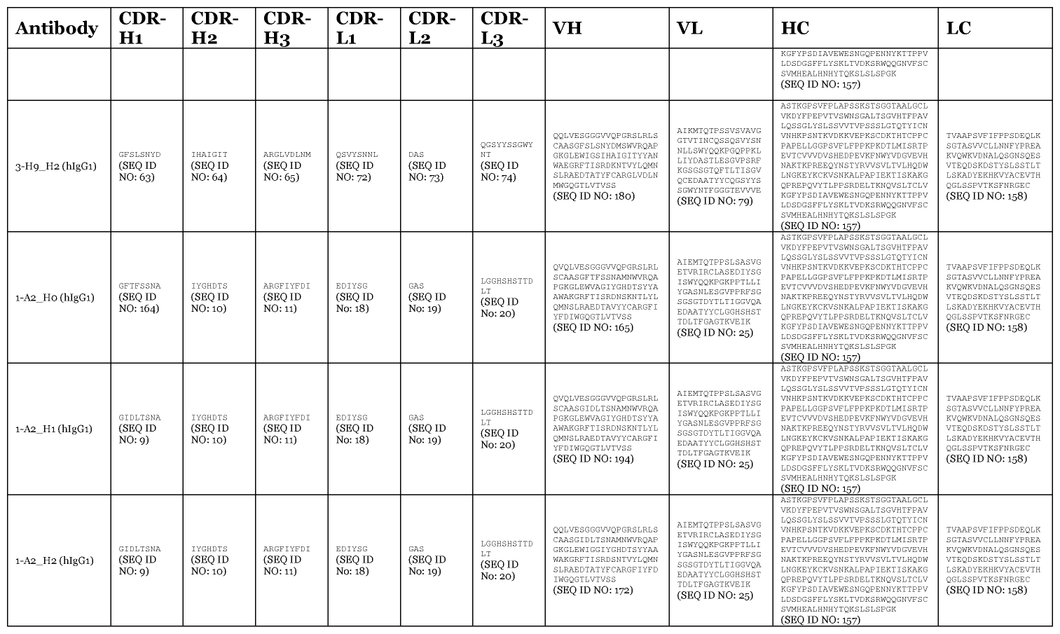

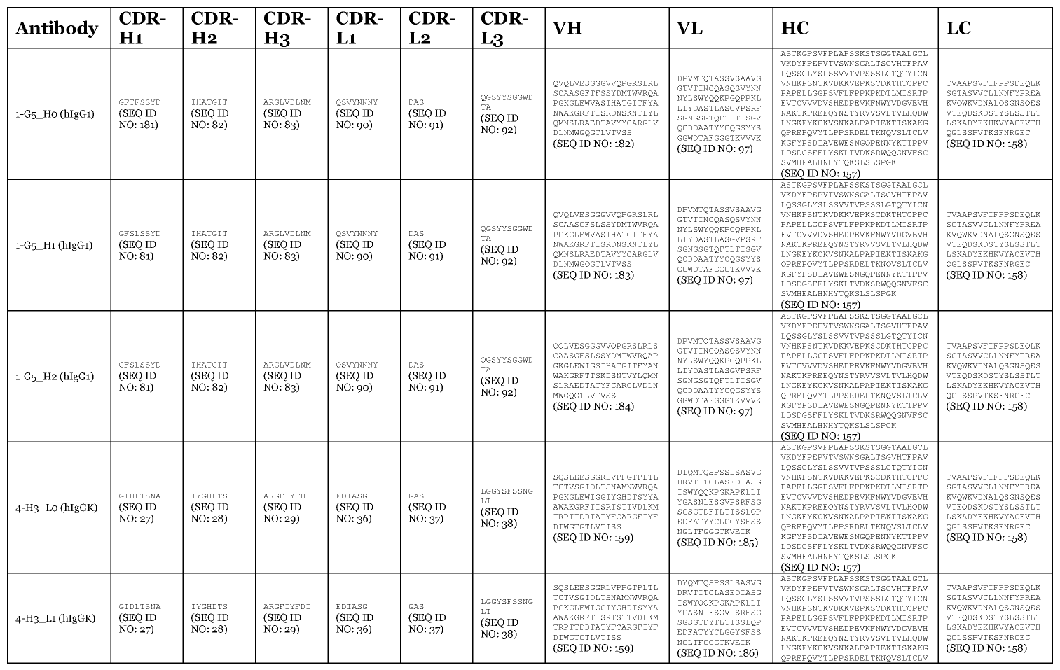

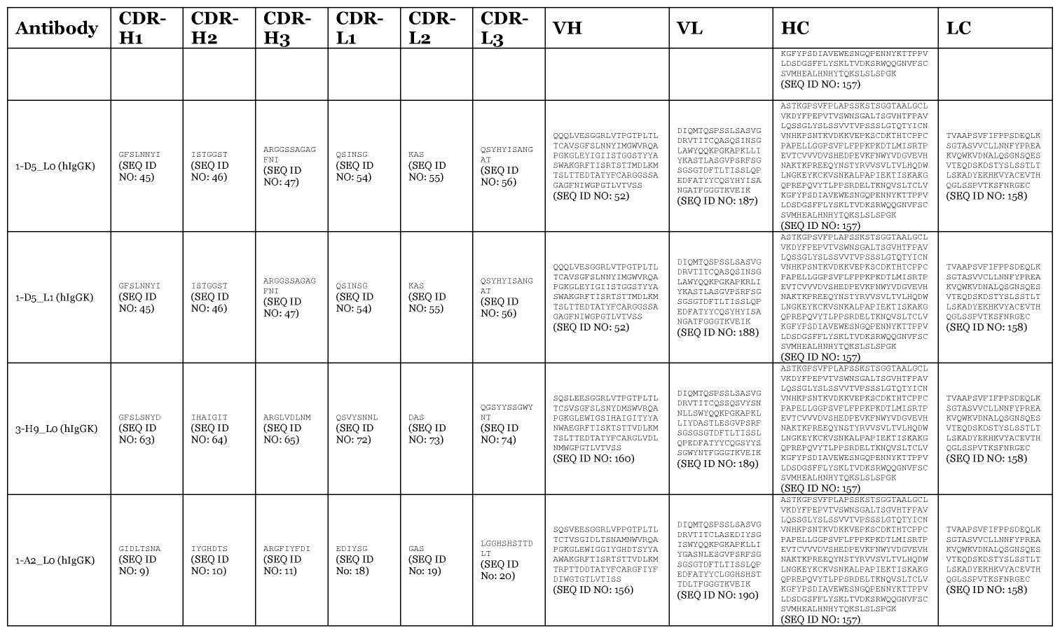

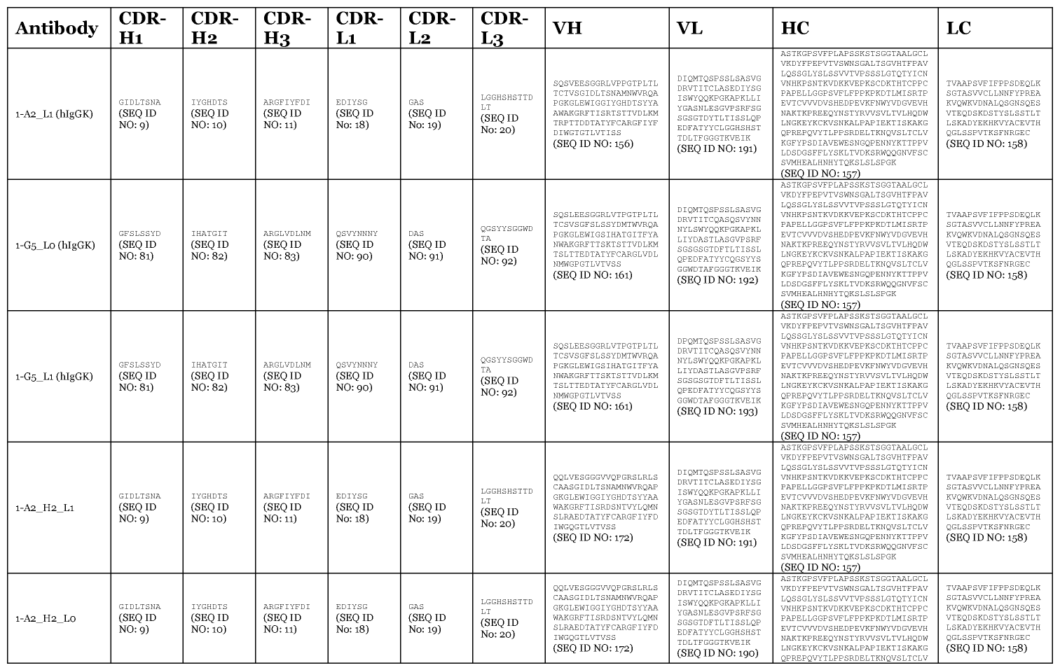

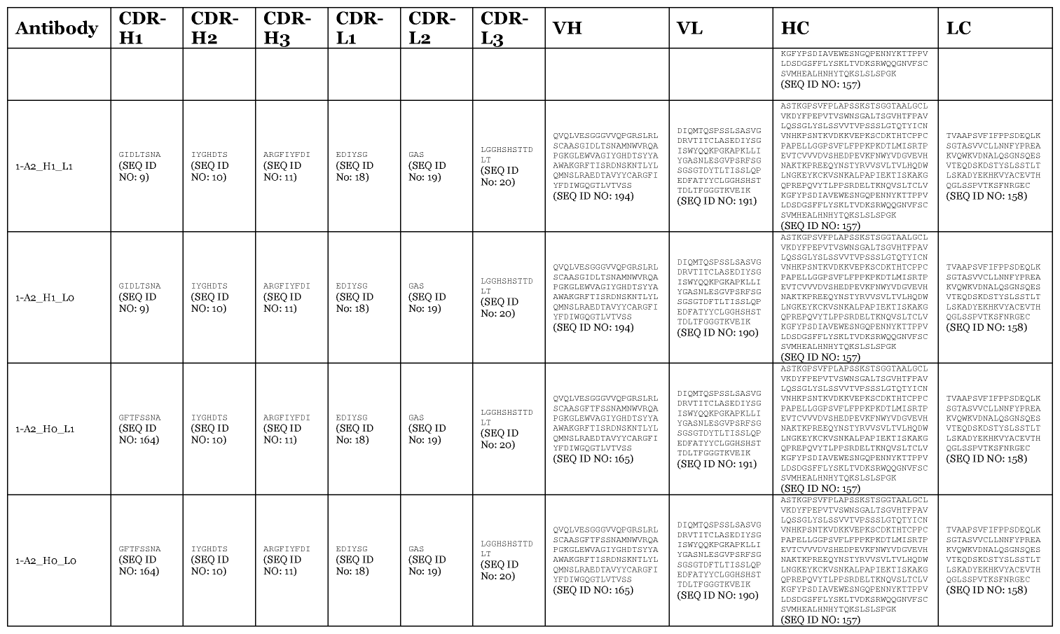

As described in the Examples and as shown in FIGS. 3A-3H and FIGS. 7A-E, the inventors have surprisingly demonstrated that the antibodies and antigen-binding fragments referred to herein as 1-A2, 4-H3, 1-D5, 3-H9, 1-G5, 4-H9, 4-B12, and 4-C6, are able to significantly target one or more of the C1-C6 domains of VWF, and each of these antibodies are defined below in detail. The CDR/FR/VH/VL, HC and LC sequences of these eight antibodies are conveniently summarised in the table shown in FIG. 8.

### 1-A2

Accordingly, in one embodiment, the antibody or antigen-binding fragment thereof is referred to herein as 1-A2. The antibody or antigen-binding fragment thereof may comprise a CDR-H1 domain of SEQ ID No: 9, which is provided herein, as follows:

[SEQ ID No: 9]
GIDLTSNA

Thus, preferably the antibody or antigen-binding fragment thereof comprises a CDR-H1 domain comprising or consisting of a sequence as substantially set out in SEQ ID No: 9, or a variant or fragment thereof.

The antibody or antigen-binding fragment thereof may comprise a CDR-H2 domain of SEQ ID No: 10, which is provided herein, as follows:

[SEQ ID No: 10]
IYGHDTS

Thus, preferably the antibody or antigen-binding fragment thereof comprises a CDR-H2 domain comprising or consisting of a sequence as substantially set out in SEQ ID No: 10, or a variant or fragment thereof.

The antibody or antigen-binding fragment thereof may comprise a CDR-H3 domain of SEQ ID No: 11, which is provided herein, as follows:

[SEQ ID No: 11]
ARGFIYFDI

Thus, preferably the antibody or antigen-binding fragment thereof comprises a CDR-H3 domain comprising or consisting of a sequence as substantially set out in SEQ ID No: 11, or a variant or fragment thereof.

Preferably, the antibody or antigen-binding fragment thereof comprises a CDR-H1 domain comprising or consisting of SEQ ID No: 9, a CDR-H2 domain comprising or consisting of SEQ ID No: 10 and/or a CDR-H3 domain comprising or consisting of SEQ ID No: 11. Preferably, however, the antibody or antigen-binding fragment thereof comprises a CDR-H1 domain comprising or consisting of SEQ ID No: 9, a CDR-H2 domain comprising or consisting of SEQ ID No: 10 and a CDR-H3 domain comprising or consisting of SEQ ID No: 11.

The antibody or antigen-binding fragment thereof may comprise a FR-H1 domain of SEQ ID No: 12, which is provided herein, as follows:

[SEQ ID No: 12]
SQSVEESGGRLVPPGTPLTLTCTVS

Thus, preferably, the antibody or antigen-binding fragment thereof comprises a FR-H1 domain comprising or consisting of a sequence as substantially set out in SEQ ID No: 12, or a variant or fragment thereof.

The antibody or antigen-binding fragment thereof may comprise a FR-H2 domain of SEQ ID No: 13, which is provided herein, as follows:

[SEQ ID No: 13]
MNWVRQAPGKGLEWIGG

Thus, preferably, the antibody or antigen-binding fragment thereof comprises a FR-H2 domain comprising or consisting of a sequence as substantially set out in SEQ ID No: 13, or a variant or fragment thereof.

The antibody or antigen-binding fragment thereof may comprise a FR-H3 domain of SEQ ID No: 14, which is provided herein, as follows:

[SEQ ID No: 14]
YYAAWAKGRFTISRTSTTVDLKMTRPTTDDTATYFC

Thus, preferably, the antibody or antigen-binding fragment thereof comprises a FR-H3 domain comprising or consisting of a sequence as substantially set out in SEQ ID No: 14, or a variant or fragment thereof.

The antibody or antigen-binding fragment thereof may comprise a FR-H4 domain of SEQ ID No: 15, which is provided herein, as follows:

[SEQ ID No: 15]
WGTGTLVTISS

Thus, preferably, the antibody or antigen-binding fragment thereof comprises a FR-H4 domain comprising or consisting of a sequence as substantially set out in SEQ ID No:, or a variant or fragment thereof.

Preferably, the antibody or antigen-binding fragment thereof comprises a FR-H1 domain comprising or consisting of SEQ ID No: 12, a FR-H2 domain comprising or consisting of SEQ ID No: 13, a FR-H3 domain comprising or consisting of SEQ ID No: 14, and/or a FR-H4 domain comprising or consisting of SEQ ID No: 15. Preferably, however, the antibody or antigen-binding fragment thereof comprises a FR-H1 domain comprising or consisting of SEQ ID No: 12, a FR-H2 domain comprising or consisting of SEQ ID No: 13, a FR-H3 domain comprising or consisting of SEQ ID No: 14, and a FR-H4 domain comprising or consisting of SEQ ID No: 15.

The antibody or antigen-binding fragment thereof may comprise a heavy chain variable (VH) sequence as set out in SEQ ID No: 16, which is provided herein, as follows:

[SEQ ID No: 16]
QSVEESGGRLVPPGTPLTLTCTVSGIDLTSNAMNWVRQAPGKGLEWIGG
IYGHDTSYYAAWAKGRFTISRTSTTVDLKMTRPTTDDTATYFCARGFIY
FDIWGTGTLVTISS

Preferably, the antibody or antigen-binding fragment thereof comprises a heavy chain variable (VH) region comprising or consisting of a sequence as substantially set out in SEQ ID No: 16, or a variant or fragment thereof.

One embodiment of the nucleotide sequence encoding the heavy chain variable (VH) region is referred to herein as SEQ ID No: 17, as follows:

[SEQ ID No: 17]
CAGTCGGTGGAGGAGTCCGGGGGTCGCCTGGTCCCGCCTGGGACACCCC
TGACACTCACCTGCACAGTCTCTGGAATCGACCTCACTAGCAATGCAAT
GAACTGGGTCCGCCAGGCTCCAGGGAAGGGGCTGGAATGGATCGGAGGC
ATTTATGGTCATGATACCTCATATTACGCGGCCTGGGCGAAAGGCCGAT
TCACCATCTCCAGAACCTCGACCACAGTGGATCTGAAAATGACCAGGCC
GACAACCGACGACACGGCCACCTATTTCTGTGCCAGAGGTTTTATTTAT
TTTGACATCTGGGGCACAGGCACCCTGGTCACCATCTCTTCA

Preferably, the antibody or antigen-binding fragment thereof comprises a heavy chain variable (VH) region encoded by a nucleic acid sequence as substantially set out in SEQ ID No: 17, or a variant or fragment thereof.

The antibody or antigen-binding fragment thereof may comprise a CDR-L1 domain of SEQ ID No: 18, which is provided herein, as follows:

[SEQ ID No: 18]
EDIYSG

Thus, preferably, the antibody or antigen-binding fragment thereof comprises a CDR-L1 domain comprising or consisting of a sequence as substantially set out in SEQ ID No: 18, or a variant or fragment thereof.

The antibody or antigen-binding fragment thereof may comprise a CDR-L2 domain of SEQ ID No: 19, which is provided herein, as follows:

[SEQ ID No: 19]
GAS

Thus, preferably, the antibody or antigen-binding fragment thereof comprises a CDR-L2 domain comprising or consisting of a sequence as substantially set out in SEQ ID No: 19, or a variant or fragment thereof.

The antibody or antigen-binding fragment thereof may comprise a CDR-L3 domain of SEQ ID No: 20, which is provided herein, as follows:

[SEQ ID No: 20]
LGGHSHSTTDLT

Thus, preferably, the antibody or antigen-binding fragment thereof comprises a CDR-L3 domain comprising or consisting of a sequence as substantially set out in SEQ ID No: 20, or a variant or fragment thereof.

Preferably, the antibody or antigen-binding fragment thereof comprises a CDR-L1 domain comprising or consisting of SEQ ID No: 18, a CDR-L2 domain comprising or consisting of SEQ ID No: 19, and/or a CDR-L3 domain comprising or consisting of SEQ ID No: 20. However, preferably the antibody or antigen-binding fragment thereof comprises a CDR-L1 domain comprising or consisting of SEQ ID No: 18, a CDR-L2 domain comprising or consisting of SEQ ID No: 19, and a CDR-L3 domain comprising or consisting of SEQ ID No: 20.

The antibody or antigen-binding fragment thereof may comprise a FR-L1 domain of SEQ ID No: 21, which is provided herein, as follows:

[SEQ ID No: 21]
AIEMTQTPPSLSASVGETVRIRCLAS

Thus, preferably, the antibody or antigen-binding fragment thereof comprises a FR-L1 domain comprising or consisting of a sequence as substantially set out in SEQ ID No: 21, or a variant or fragment thereof.

The antibody or antigen-binding fragment thereof may comprise a FR-L2 domain of SEQ ID No: 22, which is provided herein, as follows:

[SEQ ID No: 22]
ISWYQQKPGKPPTLLIY

Thus, preferably, the antibody or antigen-binding fragment thereof comprises a FR-L2 domain comprising or consisting of a sequence as substantially set out in SEQ ID No: 22, or a variant or fragment thereof.

The antibody or antigen-binding fragment thereof may comprise a FR-L3 domain of SEQ ID No: 23, which is provided herein, as follows:

[SEQ ID No: 23]
NLESGVPPRFSGSGSGTDYTLTIGGVQAEDAATYYC

Thus, preferably, the antibody or antigen-binding fragment thereof comprises a FR-L3 domain comprising or consisting of a sequence as substantially set out in SEQ ID No: 23, or a variant or fragment thereof.

The antibody or antigen-binding fragment thereof may comprise a FR-L4 domain of SEQ ID No: 24, which is provided herein, as follows:

[SEQ ID No: 24]
FGAGTKVEIK

Thus, preferably, the antibody or antigen-binding fragment thereof comprises a FR-L4 domain comprising or consisting of a sequence as substantially set out in SEQ ID No: 24, or a variant or fragment thereof.

Preferably, the antibody or antigen-binding fragment thereof comprises a FR-L1 domain comprising or consisting of SEQ ID No: 21, a FR-L2 domain comprising or consisting of SEQ ID No: 22, a FR-L3 domain comprising or consisting of SEQ ID No: 23, and/or a FR-L4 domain comprising or consisting of SEQ ID No: 24. Preferably, however, the antibody or antigen-binding fragment thereof comprises a FR-L1 domain comprising or consisting of SEQ ID No: 21, a FR-L2 domain comprising or consisting of SEQ ID No: 22, a FR-L3 domain comprising or consisting of SEQ ID No: 23, and a FR-L4 domain comprising or consisting of SEQ ID No: 24.

The antibody or antigen-binding fragment thereof may comprise a light chain variable (VL) sequence as set out in SEQ ID No: 25, which is provided herein, as follows:

[SEQ ID No: 25]
AIEMTQTPPSLSASVGETVRIRCLASEDIYSGISWYQQKPGKPPTLLIY
GASNLESGVPPRFSGSGSGTDYTLTIGGVQAEDAATYYCLGGHSHSTTD
LTFGAGTKVEIK

Preferably, the antibody or antigen-binding fragment thereof comprises a light chain variable region comprising or consisting of a sequence as substantially set out in SEQ ID No: 25, or a variant or fragment thereof.

One embodiment of the nucleotide sequence encoding the light chain variable (VL) region is referred to herein as SEQ ID No: 26, as follows:

[SEQ ID No: 26]
GCAATTGAGATGACCCAGACTCCACCCTCCCTGTCTGCATCTGTGGGAG
AAACTGTCAGGATTAGGTGCCTGGCCAGTGAGGACATTTACAGTGGTAT
ATCCTGGTATCAACAGAAGCCAGGGAAACCTCCTACACTCCTGATCTAT
GGTGCATCCAATTTAGAATCTGGGGTCCCACCACGGTTCAGTGGCAGTG
GATCTGGGACAGATTACACCCTCACCATTGGCGGCGTGCAGGCTGAAGA
TGCTGCCACCTACTACTGTCTAGGCGGTCATAGCCACAGTACTACCGAT
TTGACTTTTGGAGCTGGGACCAAGGTGGAAATCAAA

Preferably, the antibody or antigen-binding fragment thereof comprises a light chain variable (VL) region encoded by a nucleic acid sequence as substantially set out in SEQ ID No: 26, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 9, a CDR-H2 domain comprising or consisting of SEQ ID No: 10; a CDR-H3 domain comprising or consisting of SEQ ID No: 11, a CDR-L1 domain comprising or consisting of SEQ ID No: 18, a CDR-L2 domain comprising or consisting of SEQ ID No: 19, and a CDR-L3 domain comprising or consisting of SEQ ID No: 20.

Preferably, the antibody or antigen-binding fragment thereof comprises a heavy chain variable region comprising or consisting of SEQ ID No: 16, and a light chain variable region comprising or consisting of SEQ ID No: 25.

Preferably, the antibody or antigen-binding fragment thereof comprises a heavy chain variable region encoded by a nucleic acid sequence comprising or consisting of SEQ ID No: 17, and a light chain variable region encoded by a nucleic acid sequence comprising or consisting of SEQ ID No: 26.

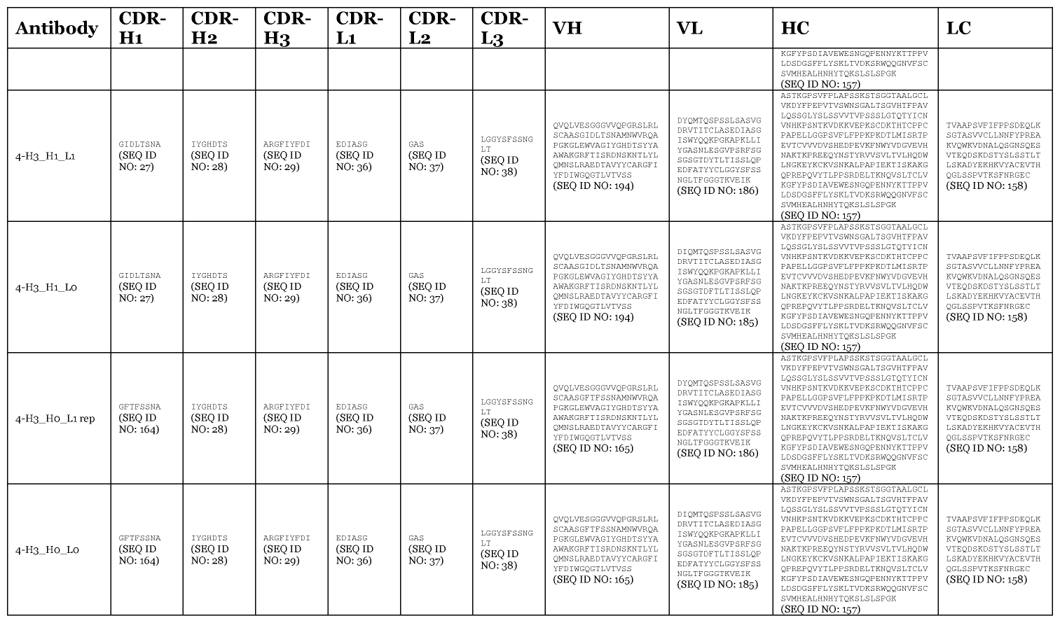

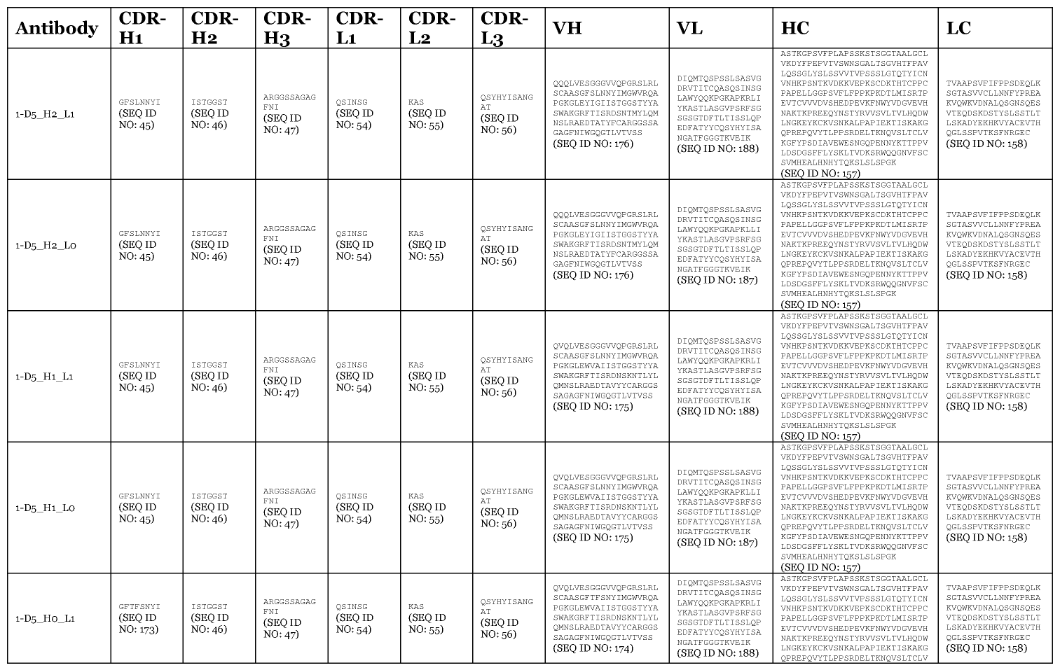

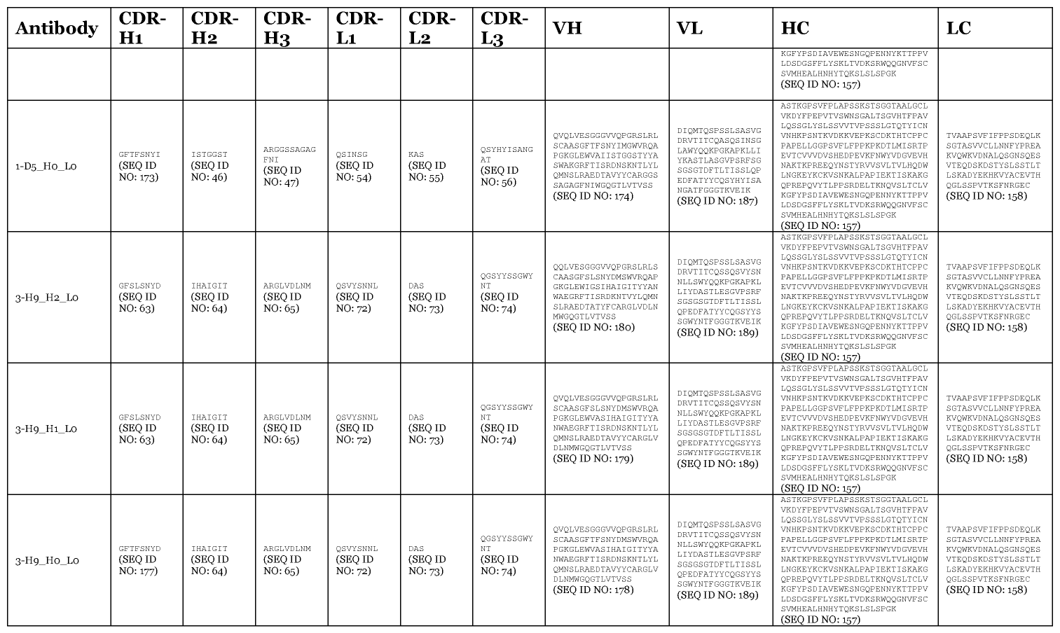

The inventors then set out to generate humanised antibodies of 1-A2, and the sequences of the humanised antibodies are illustrated in FIG. 10. Unless stated otherwise, the six CDR sequences of the humanised antibodies are identical to the six CDR sequences of the parental antibody 1-A2.

1-A2 Parental (hIgG1)

In one embodiment, the humanised antibody or antigen-binding fragment thereof is referred to herein as 1-A2_parental (hIgG1).

Preferably, the antibody or antigen-binding fragment thereof comprises a heavy chain variable (VH) region comprising or consisting of a sequence as substantially set out in SEQ ID No: 156, or a variant or fragment thereof.

Preferably, the antibody or antigen-binding fragment thereof comprises a light chain variable (VL) region comprising or consisting of a sequence as substantially set out in SEQ ID No: 25, or a variant or fragment thereof.

Preferably, the antibody or antigen-binding fragment thereof comprises a heavy chain variable region comprising or consisting of SEQ ID No: 156 and a light chain variable region comprising or consisting of SEQ ID No: 25.

Preferably, the antibody or antigen-binding fragment thereof comprises a heavy chain constant (HC) region comprising or consisting of a sequence as substantially set out in

SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

1-A2 Parental (hIgG1-L234A-L235A-P329G)

Alternatively, in another embodiment, the humanised antibody or antigen-binding fragment thereof is referred to herein as 1-A2_parental (hIgG1-L234A-L235A-P329G).

Preferably, the antibody or antigen-binding fragment thereof comprises a heavy chain variable (VH) region comprising or consisting of a sequence as substantially set out in SEQ ID No: 156, or a variant or fragment thereof.

Preferably, the antibody or antigen-binding fragment thereof comprises a light chain variable (VL) region comprising or consisting of a sequence as substantially set out in SEQ ID No: 25, or a variant or fragment thereof.

Preferably, the antibody or antigen-binding fragment thereof comprises a heavy chain variable region comprising or consisting of SEQ ID No: 156 and a light chain variable region comprising or consisting of SEQ ID No: 25.

Preferably, the antibody or antigen-binding fragment thereof comprises a heavy chain constant (HC) region comprising or consisting of a sequence as substantially set out in SEQ ID No: 162, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 162 and a light chain constant region comprising or consisting of SEQ ID No: 158.

1-A2 Parental (hIgG1-Fab)

Alternatively, in another embodiment, the humanised antibody or antigen-binding fragment thereof is referred to herein as 1-A2_parental (hIgG1-Fab).

Preferably, the antibody or antigen-binding fragment thereof comprises a heavy chain variable (VH) region comprising or consisting of a sequence as substantially set out in SEQ ID No: 156, or a variant or fragment thereof.

Preferably, the antibody or antigen-binding fragment thereof comprises a light chain variable (VL) region comprising or consisting of a sequence as substantially set out in SEQ ID No: 25, or a variant or fragment thereof.

Preferably, the antibody or antigen-binding fragment thereof comprises a heavy chain variable region comprising or consisting of SEQ ID No: 156 and a light chain variable region comprising or consisting of SEQ ID No: 25.

Preferably, the antibody or antigen-binding fragment thereof comprises a heavy chain constant (HC) region comprising or consisting of a sequence as substantially set out in SEQ ID No: 163, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 163 and a light chain constant region comprising or consisting of SEQ ID No: 158.

1-A2 H0 (hIgG1)

Alternatively, in another embodiment, the humanised antibody or antigen-binding fragment thereof is referred to herein as 1-A2_H0 (hIgG1).

Preferably, the antibody or antigen-binding fragment thereof comprises a CDR-H1 domain comprising or consisting of a sequence as substantially set out in SEQ ID No: 164, or a variant or fragment thereof. Preferably, the antibody or antigen-binding fragment thereof comprises a CDR-H2 domain comprising or consisting of a sequence as substantially set out in SEQ ID No: 10, or a variant or fragment thereof. Preferably, the antibody or antigen-binding fragment thereof comprises a CDR-H3 domain comprising or consisting of a sequence as substantially set out in SEQ ID No: 11, or a variant or fragment thereof.

Preferably, the antibody or antigen-binding fragment thereof comprises a CDR-L1 domain comprising or consisting of a sequence as substantially set out in SEQ ID No: 18, or a variant or fragment thereof. Preferably, the antibody or antigen-binding fragment thereof comprises a CDR-L2 domain comprising or consisting of a sequence as substantially set out in SEQ ID No: 19, or a variant or fragment thereof. Preferably, the antibody or antigen-binding fragment thereof comprises a CDR-L3 domain comprising or consisting of a sequence as substantially set out in SEQ ID No: 20, or a variant or fragment thereof.

Preferably, the antibody or antigen-binding fragment thereof comprises a CDR-H1 domain comprising or consisting of SEQ ID No: 164, a CDR-H2 domain comprising or consisting of SEQ ID No: 10; a CDR-H3 domain comprising or consisting of SEQ ID No: 11, a CDR-L1 domain comprising or consisting of SEQ ID No: 18, a CDR-L2 domain comprising or consisting of SEQ ID No: 19, and a CDR-L3 domain comprising or consisting of SEQ ID No: 20.

Preferably, the antibody or antigen-binding fragment thereof comprises a heavy chain variable (VH) region comprising or consisting of a sequence as substantially set out in SEQ ID No: 165, or a variant or fragment thereof.

Preferably, the antibody or antigen-binding fragment thereof comprises a light chain variable (VL) region comprising or consisting of a sequence as substantially set out in SEQ ID No: 25, or a variant or fragment thereof.

Preferably, the antibody or antigen-binding fragment thereof comprises a heavy chain variable region comprising or consisting of SEQ ID No: 165 and a light chain variable region comprising or consisting of SEQ ID No: 25.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in

SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

1-A2 H1 (hIgG1)

Alternatively, in another embodiment, the humanised antibody or antigen-binding fragment thereof is referred to herein as 1-A2_H1 (hIgG1).

Preferably, the antibody or antigen-binding fragment thereof comprises a heavy chain variable (VH) region comprising or consisting of a sequence as substantially set out in SEQ ID No: 194, or a variant or fragment thereof.

Preferably, the antibody or antigen-binding fragment thereof comprises a light chain variable (VL) region comprising or consisting of a sequence as substantially set out in SEQ ID No: 25, or a variant or fragment thereof.

Preferably, the antibody or antigen-binding fragment thereof comprises a heavy chain variable region comprising or consisting of SEQ ID No: 194 and a light chain variable region comprising or consisting of SEQ ID No: 25.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

1-A2 H2 (hIgG1)

Alternatively, in another embodiment, the humanised antibody or antigen-binding fragment thereof is referred to herein as 1-A2_H2 (hIgG1).

Preferably, the antibody or antigen-binding fragment thereof comprises a heavy chain variable (VH) region comprising or consisting of a sequence as substantially set out in SEQ ID No: 172, or a variant or fragment thereof.

Preferably, the antibody or antigen-binding fragment thereof comprises a light chain variable (VL) region comprising or consisting of a sequence as substantially set out in SEQ ID No: 25, or a variant or fragment thereof.

Preferably, the antibody or antigen-binding fragment thereof comprises a heavy chain variable region comprising or consisting of SEQ ID No: 172 and a light chain variable region comprising or consisting of SEQ ID No: 25.

Preferably, the antibody or antigen-binding fragment thereof comprises a heavy chain constant (HC) region comprising or consisting of a sequence as substantially set out in SEQ ID NO: 157, or a varient or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

1-A2 L0 (hIgG1K)

Alternatively, in another embodiment, the humanised antibody or antigen-binding fragment thereof is referred to herein as 1-A2_L0 (hIgG1K).

Preferably, the antibody or antigen-binding fragment thereof comprises a heavy chain variable (VH) region comprising or consisting of a sequence as substantially set out in SEQ ID No: 156, or a variant or fragment thereof.

Preferably, the antibody or antigen-binding fragment thereof comprises a light chain variable (VL) region comprising or consisting of a sequence as substantially set out in

SEQ ID No: 190, or a variant or fragment thereof.

Preferably, the antibody or antigen-binding fragment thereof comprises a heavy chain variable region comprising or consisting of SEQ ID No: 156 and a light chain variable region comprising or consisting of SEQ ID No: 190.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

1-A2 L1 (hIgG1K)

Alternatively, in another embodiment, the humanised antibody or antigen-binding fragment thereof is referred to herein as 1-A2_L1 (hIgG1K).

Preferably, the antibody or antigen-binding fragment thereof comprises a heavy chain variable (VH) region comprising or consisting of a sequence as substantially set out in SEQ ID No: 156, or a variant or fragment thereof.

Preferably, the antibody or antigen-binding fragment thereof comprises a light chain variable (VL) region comprising or consisting of a sequence as substantially set out in SEQ ID No: 191, or a variant or fragment thereof.

Preferably, the antibody or antigen-binding fragment thereof comprises a heavy chain variable region comprising or consisting of SEQ ID No: 156 and a light chain variable region comprising or consisting of SEQ ID No: 191.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-A2 H2 L1

Alternatively, in another embodiment, the humanised antibody or antigen-binding fragment thereof is referred to herein as 1-A2_H2_L1.

Preferably, the antibody or antigen-binding fragment thereof comprises a heavy chain variable (VH) region comprising or consisting of a sequence as substantially set out in SEQ ID No: 172, or a variant or fragment thereof.

Preferably, the antibody or antigen-binding fragment thereof comprises a light chain variable (VL) region comprising or consisting of a sequence as substantially set out in SEQ ID No: 191, or a variant or fragment thereof.

Preferably, the antibody or antigen-binding fragment thereof comprises a heavy chain variable region comprising or consisting of SEQ ID No: 172 and a light chain variable region comprising or consisting of SEQ ID No: 191.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-A2 H2 L0

Alternatively, in another embodiment, the humanised antibody or antigen-binding fragment thereof is referred to herein as 1-A2_H2_L0.

Preferably, the antibody or antigen-binding fragment thereof comprises a heavy chain variable (VH) region comprising or consisting of a sequence as substantially set out in SEQ ID No: 172, or a variant or fragment thereof.

Preferably, the antibody or antigen-binding fragment thereof comprises a light chain variable (VL) region comprising or consisting of a sequence as substantially set out in SEQ ID No: 190, or a variant or fragment thereof.

Preferably, the antibody or antigen-binding fragment thereof comprises a heavy chain variable region comprising or consisting of SEQ ID No: 172 and a light chain variable region comprising or consisting of SEQ ID No: 190.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-A2 H1 L1

Alternatively, in another embodiment, the humanised antibody or antigen-binding fragment thereof is referred to herein as 1-A2_H1_L1.

Preferably, the antibody or antigen-binding fragment thereof comprises a heavy chain variable (VH) region comprising or consisting of a sequence as substantially set out in

SEQ ID No: 194, or a variant or fragment thereof.

Preferably, the antibody or antigen-binding fragment thereof comprises a light chain variable (VL) region comprising or consisting of a sequence as substantially set out in SEQ ID No: 191, or a variant or fragment thereof.

Preferably, the antibody or antigen-binding fragment thereof comprises a heavy chain variable region comprising or consisting of SEQ ID No: 194 and a light chain variable region comprising or consisting of SEQ ID No: 191.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 58, or variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-A2 H1 L0

Alternatively, in another embodiment, the humanised antibody or antigen-binding fragment thereof is referred to herein as 1-A2_H1_L0.

Preferably, the antibody or antigen-binding fragment thereof comprises a heavy chain variable (VH) region comprising or consisting of a sequence as substantially set out in SEQ ID No: 194, or a variant or fragment thereof.

Preferably, the antibody or antigen-binding fragment thereof comprises a light chain variable (VL) region comprising or consisting of a sequence as substantially set out in SEQ ID No: 190, or a variant or fragment thereof.

Preferably, the antibody or antigen-binding fragment thereof comprises a heavy chain variable region comprising or consisting of SEQ ID No: 194 and a light chain variable region comprising or consisting of SEQ ID No: 190.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-A2 H0 L1

Alternatively, in another embodiment, the humanised antibody or antigen-binding fragment thereof is referred to herein as 1-A2_H0_L1.

Preferably, the antibody or antigen-binding fragment thereof comprises a CDR-H1 domain comprising or consisting of a sequence as substantially set out in SEQ ID No: 164, or a variant or fragment thereof. Preferably, the antibody or antigen-binding fragment thereof comprises a CDR-H2 domain comprising or consisting of a sequence as substantially set out in SEQ ID No: 10, or a variant or fragment thereof. Preferably, the antibody or antigen-binding fragment thereof comprises a CDR-H3 domain comprising or consisting of a sequence as substantially set out in SEQ ID No: 11, or a variant or fragment thereof.

Preferably, the antibody or antigen-binding fragment thereof comprises a CDR-L1 domain comprising or consisting of a sequence as substantially set out in SEQ ID No: 18, or a variant or fragment thereof. Preferably, the antibody or antigen-binding fragment thereof comprises a CDR-L2 domain comprising or consisting of a sequence as substantially set out in SEQ ID No: 19, or a variant or fragment thereof. Preferably, the antibody or antigen-binding fragment thereof comprises a CDR-L3 domain comprising or consisting of a sequence as substantially set out in SEQ ID No: 20, or a variant or fragment thereof.

Preferably, the antibody or antigen-binding fragment thereof comprises a CDR-H1 domain comprising or consisting of SEQ ID No: 164, a CDR-H2 domain comprising or consisting of SEQ ID No: 10; a CDR-H3 domain comprising or consisting of SEQ ID No: 11, a CDR-L1 domain comprising or consisting of SEQ ID No: 18, a CDR-L2 domain comprising or consisting of SEQ ID No: 19, and a CDR-L3 domain comprising or consisting of SEQ ID No: 20.

Preferably, the antibody or antigen-binding fragment thereof comprises a heavy chain variable (VH) region comprising or consisting of a sequence as substantially set out in SEQ ID No: 165, or a variant or fragment thereof.

Preferably, the antibody or antigen-binding fragment thereof comprises a light chain variable (VL) region comprising or consisting of a sequence as substantially set out in SEQ ID No: 191, or a variant or fragment thereof.

Preferably, the antibody or antigen-binding fragment thereof comprises a heavy chain variable region comprising or consisting of SEQ ID No: 165 and a light chain variable region comprising or consisting of SEQ ID No: 191.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-A2 H0 L0

Alternatively, in another embodiment, the humanised antibody or antigen-binding fragment thereof is referred to herein as 1-A2_H0_L0.

Preferably, the antibody or antigen-binding fragment thereof comprises a CDR-H1 domain comprising or consisting of a sequence as substantially set out in SEQ ID No: 164, or a variant or fragment thereof. Preferably, the antibody or antigen-binding fragment thereof comprises a CDR-H2 domain comprising or consisting of a sequence as substantially set out in SEQ ID No: 10, or a variant or fragment thereof. Preferably, the antibody or antigen-binding fragment thereof comprises a CDR-H3 domain comprising or consisting of a sequence as substantially set out in SEQ ID No: 11, or a variant or fragment thereof.

Preferably, the antibody or antigen-binding fragment thereof comprises a CDR-L1 domain comprising or consisting of a sequence as substantially set out in SEQ ID No: 18, or a variant or fragment thereof. Preferably, the antibody or antigen-binding fragment thereof comprises a CDR-L2 domain comprising or consisting of a sequence as substantially set out in SEQ ID No: 19, or a variant or fragment thereof. Preferably, the antibody or antigen-binding fragment thereof comprises a CDR-L3 domain comprising or consisting of a sequence as substantially set out in SEQ ID No: 20, or a variant or fragment thereof.

Preferably, the antibody or antigen-binding fragment thereof comprises a CDR-H1 domain comprising or consisting of SEQ ID No: 164, a CDR-H2 domain comprising or consisting of SEQ ID No: 10; a CDR-H3 domain comprising or consisting of SEQ ID No: 11, a CDR-L1 domain comprising or consisting of SEQ ID No: 18, a CDR-L2 domain comprising or consisting of SEQ ID No: 19, and a CDR-L3 domain comprising or consisting of SEQ ID No: 20.

Preferably, the antibody or antigen-binding fragment thereof comprises a heavy chain variable (VH) region comprising or consisting of a sequence as substantially set out in SEQ ID No: 165, or a variant or fragment thereof.

Preferably, the antibody or antigen-binding fragment thereof comprises a light chain variable (VL) region comprising or consisting of a sequence as substantially set out in SEQ ID No: 190, or a variant or fragment thereof.

Preferably, the antibody or antigen-binding fragment thereof comprises a heavy chain variable region comprising or consisting of SEQ ID No: 165 and a light chain variable region comprising or consisting of SEQ ID No: 190.

Preferably, the antibody or antigen-binding fragment thereof comprises a heavy chain constant (HC) region comprising or consisting of a sequence as substantially set out in

SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain

### 4-H3

In one embodiment, the antibody or antigen-binding fragment thereof is referred to herein as 4-H3. The antibody or antigen-binding fragment thereof may comprise a CDR-H1 domain of SEQ ID No: 27, which is provided herein, as follows:

[SEQ ID No: 27]
GIDLTSNA

Thus, preferably, the antibody or antigen-binding fragment thereof comprises a CDR-H1 domain comprising or consisting of a sequence as substantially set out in SEQ ID

No: 27, or a variant or fragment thereof.

The antibody or antigen-binding fragment thereof may comprise a CDR-H2 domain of SEQ ID No: 28, which is provided herein, as follows:

[SEQ ID No: 28]
IYGHDTS

Thus, preferably the antibody or antigen-binding fragment thereof comprises a CDR-H2 domain comprising or consisting of a sequence as substantially set out in SEQ ID No: 28, or a variant or fragment thereof.

The antibody or antigen-binding fragment thereof may comprise a CDR-H3 domain of SEQ ID No: 29, which is provided herein, as follows:

[SEQ ID No: 29]
ARGFIYFDI

Thus, preferably the antibody or antigen-binding fragment thereof comprises a CDR-H3 domain comprising or consisting of a sequence as substantially set out in SEQ ID No: 29, or a variant or fragment thereof.

Preferably, the antibody or antigen-binding fragment thereof comprises a CDR-H1 domain comprising or consisting of SEQ ID No: 27, a CDR-H2 domain comprising or consisting of SEQ ID No: 28 and/or a CDR-H3 domain comprising or consisting of SEQ

ID No: 29. Preferably, however, the antibody or antigen-binding fragment thereof comprises a CDR-H1 domain comprising or consisting of SEQ ID No: 27, a CDR-H2 domain comprising or consisting of SEQ ID No: 28 and a CDR-H3 domain comprising or consisting of SEQ ID No: 29.

The antibody or antigen-binding fragment thereof may comprise a FR-H1 domain of SEQ ID No: 30, which is provided herein, as follows:

[SEQ ID No: 30]
SQSLEESGGRLVPPGTPLTLTCTVS

Thus, preferably, the antibody or antigen-binding fragment thereof comprises a FR-H1 domain comprising or consisting of a sequence as substantially set out in SEQ ID No: 30, or a variant or fragment thereof.

The antibody or antigen-binding fragment thereof may comprise a FR-H2 domain of SEQ ID No: 31, which is provided herein, as follows:

[SEQ ID No: 31]
MNWVRQAPGKGLEWIGG

Thus, preferably, the antibody or antigen-binding fragment thereof comprises a FR-H2 domain comprising or consisting of a sequence as substantially set out in SEQ ID No: 31, or a variant or fragment thereof.

The antibody or antigen-binding fragment thereof may comprise a FR-H3 domain of SEQ ID No: 32, which is provided herein, as follows:

[SEQ ID No: 32]
YYAAWAKGRFTISRTSTTVDLKMTRPTTDDTATYFC

Thus, preferably, the antibody or antigen-binding fragment thereof comprises a FR-H3 domain comprising or consisting of a sequence as substantially set out in SEQ ID No: 32, or a variant or fragment thereof.

The antibody or antigen-binding fragment thereof may comprise a FR-H4 domain of SEQ ID No: 33, which is provided herein, as follows:

[SEQ ID No: 33]
WGTGTLVTISS

Thus, preferably, the antibody or antigen-binding fragment thereof comprises a FR-H4 domain comprising or consisting of a sequence as substantially set out in SEQ ID No: 33, or a variant or fragment thereof.

Preferably, the antibody or antigen-binding fragment thereof comprises a FR-H1 domain comprising or consisting of SEQ ID No: 30, a FR-H2 domain comprising or consisting of SEQ ID No: 31, a FR-H3 domain comprising or consisting of SEQ ID No: 32, and/or a FR-H4 domain comprising or consisting of SEQ ID No: 33. Preferably, however, the antibody or antigen-binding fragment thereof comprises a FR-H1 domain comprising or consisting of SEQ ID No: 30, a FR-H2 domain comprising or consisting of SEQ ID No: 31, a FR-H3 domain comprising or consisting of SEQ ID No: 32, and a FR-H4 domain comprising or consisting of SEQ ID No: 33.

The antibody or antigen-binding fragment thereof may comprise a heavy chain variable (VH) sequence as set out in SEQ ID No: 34, which is provided herein, as follows:

[SEQ ID No: 34]
QSLEESGGRLVPPGTPLTLTCTVSGIDLTSNAMNWVRQAPGKGLEWIGG
IYGHDTSYYAAWAKGRFTISRTSTTVDLKMTRPTTDDTATYFCARGFIY
FDIWGTGTLVTISS

Preferably, the antibody or antigen-binding fragment thereof comprises a heavy chain variable (VH) region comprising or consisting of a sequence as substantially set out in SEQ ID No: 34, or a variant or fragment thereof.

One embodiment of the nucleotide sequence encoding the heavy chain variable (VH) region is referred to herein as SEQ ID No: 35, as follows:

[SEQ ID No: 35]
CAGTCGCTGGAGGAGTCCGGGGGTCGCCTGGTCCCGCCTGGGACACCCC
TGACACTCACCTGCACAGTCTCTGGAATCGACCTCACTAGCAATGCAAT
GAACTGGGTCCGCCAGGCTCCAGGGAAGGGGCTGGAATGGATCGGAGGC
ATTTATGGTCATGATACCTCATATTACGCGGCCTGGGCGAAAGGCCGAT
TCACCATCTCCAGAACCTCGACCACAGTGGATCTGAAAATGACCAGGCC
GACAACCGACGACACGGCCACCTATTTCTGTGCCAGAGGTTTTATTTAT
TTTGACATCTGGGGCACAGGCACCCTGGTCACCATCTCTTCA

Preferably, the antibody or antigen-binding fragment thereof comprises a heavy chain variable (VH) region encoded by a nucleic acid sequence as substantially set out in SEQ ID No: 35, or a variant or fragment thereof.

The antibody or antigen-binding fragment thereof may comprise a CDR-L1 domain of SEQ ID No: 36, which is provided herein, as follows:

[SEQ ID No: 36]
EDIASG

Thus, preferably, the antibody or antigen-binding fragment thereof comprises a CDR-L1 domain comprising or consisting of a sequence as substantially set out in SEQ ID No: 36, or a variant or fragment thereof.

The antibody or antigen-binding fragment thereof may comprise a CDR-L2 domain of SEQ ID No: 37, which is provided herein, as follows:

[SEQ ID No: 37]
GAS

Thus, preferably, the antibody or antigen-binding fragment thereof comprises a CDR-L2 domain comprising or consisting of a sequence as substantially set out in SEQ ID No: 37, or a variant or fragment thereof.

The antibody or antigen-binding fragment thereof may comprise a CDR-L3 domain of SEQ ID No: 38, which is provided herein, as follows:

[SEQ ID No: 38]
LGGYSFSSNGLT

Thus, preferably, the antibody or antigen-binding fragment thereof comprises a CDR-L3 domain comprising or consisting of a sequence as substantially set out in SEQ ID No: 38, or a variant or fragment thereof.

Preferably, the antibody or antigen-binding fragment thereof comprises a CDR-L1 domain comprising or consisting of SEQ ID No: 36, a CDR-L2 domain comprising or consisting of SEQ ID No: 37, and/or a CDR-L3 domain comprising or consisting of SEQ ID No: 38. However, preferably the antibody or antigen-binding fragment thereof comprises a CDR-L1 domain comprising or consisting of SEQ ID No: 36, a CDR-L2 domain comprising or consisting of SEQ ID No: 37, and a CDR-L3 domain comprising or consisting of SEQ ID No: 38.

The antibody or antigen-binding fragment thereof may comprise a FR-L1 domain of SEQ ID No: 39, which is provided herein, as follows:

[SEQ ID No: 39]
AYDMTQTPPSLSASVGETVRIRCLAS

Thus, preferably, the antibody or antigen-binding fragment thereof comprises a FR-L1 domain comprising or consisting of a sequence as substantially set out in SEQ ID No: 39, or a variant or fragment thereof.

The antibody or antigen-binding fragment thereof may comprise a FR-L2 domain of SEQ ID No: 40, which is provided herein, as follows:

[SEQ ID No: 40]
ISWYQQKPGKPPTLLIY

Thus, preferably, the antibody or antigen-binding fragment thereof comprises a FR-L2 domain comprising or consisting of a sequence as substantially set out in SEQ ID No: 40, or a variant or fragment thereof.

The antibody or antigen-binding fragment thereof may comprise a FR-L3 domain of SEQ ID No: 41, which is provided herein, as follows:

[SEQ ID No: 41]
NLESGVPPRFSGSGSGTDYTLTIGGVQAEDAATYYC

Thus, preferably, the antibody or antigen-binding fragment thereof comprises a FR-L3 domain comprising or consisting of a sequence as substantially set out in SEQ ID No: 41, or a variant or fragment thereof.

The antibody or antigen-binding fragment thereof may comprise a FR-L4 domain of SEQ ID No: 42, which is provided herein, as follows:

[SEQ ID No: 42]
FGAGTKVEIK

Thus, preferably, the antibody or antigen-binding fragment thereof comprises a FR-L4 domain comprising or consisting of a sequence as substantially set out in SEQ ID No: 42, or a variant or fragment thereof.

Preferably, the antibody or antigen-binding fragment thereof comprises a FR-L1 domain comprising or consisting of SEQ ID No: 39, a FR-L2 domain comprising or consisting of SEQ ID No: 40, a FR-L3 domain comprising or consisting of SEQ ID No: 41, and/or a FR-L4 domain comprising or consisting of SEQ ID No: 42. Preferably, however, the antibody or antigen-binding fragment thereof comprises a FR-L1 domain comprising or consisting of SEQ ID No: 39, a FR-L2 domain comprising or consisting of SEQ ID No: 40, a FR-L3 domain comprising or consisting of SEQ ID No: 41, and a FR-L4 domain comprising or consisting of SEQ ID No: 42.

The antibody or antigen-binding fragment thereof may comprise a light chain variable (VL) sequence as set out in SEQ ID No: 43, which is provided herein, as follows:

[SEQ ID No: 43]
AYDMTQTPPSLSASVGETVRIRCLASEDIASGISWYQQKPGKPPTLLIY
GASNLESGVPPRFSGSGSGTDYTLTIGGVQAEDAATYYCLGGYSFSSNG
LTFGAGTKVEIK

Preferably, the antibody or antigen-binding fragment thereof comprises a light chain variable region comprising or consisting of a sequence as substantially set out in SEQ ID No: 43, or a variant or fragment thereof.

One embodiment of the nucleotide sequence encoding the light chain variable (VL) region is referred to herein as SEQ ID No: 44, as follows:

[SEQ ID No: 44]
GCTTATGATATGACCCAGACTCCACCCTCCCTGTCTGCATCTGTGGGAG
AAACTGTCAGGATTAGGTGCCTGGCCAGTGAGGACATTGCCAGTGGTAT
ATCCTGGTATCAACAGAAGCCAGGGAAACCTCCTACACTCCTGATCTAT
GGTGCATCCAATTTAGAATCTGGGGTCCCACCACGGTTCAGTGGCAGTG
GATCTGGGACAGATTACACCCTCACCATTGGCGGCGTGCAGGCTGAAGA
TGCTGCCACCTACTACTGTCTAGGCGGTTATAGTTTCAGTAGTAACGGT
TTGACTTTTGGAGCTGGCACCAAGGTGGAGATCAAA

Preferably, the antibody or antigen-binding fragment thereof comprises a light chain variable (VL) region encoded by a nucleic acid sequence as substantially set out in SEQ ID No: 44, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 27, a CDR-H2 domain comprising or consisting of SEQ ID No: 28; a CDR-H3 domain comprising or consisting of SEQ ID No: 29, a CDR-L1 domain comprising or consisting of SEQ ID No: 36, a CDR-L2 domain comprising or consisting of SEQ ID No: 37, and a CDR-L3 domain comprising or consisting of SEQ ID No: 38.

Preferably, the antibody or antigen-binding fragment thereof comprises a heavy chain variable region comprising or consisting of SEQ ID No: 34, and a light chain variable region comprising or consisting of SEQ ID No: 43.

Preferably, the antibody or antigen-binding fragment thereof comprises a heavy chain variable region encoded by a nucleic acid sequence comprising or consisting of SEQ ID No: 35, and a light chain variable region encoded by a nucleic acid sequence comprising or consisting of SEQ ID No: 44.

The inventors then set out to generate humanised antibodies of 4-H3, and the sequences of the humanised antibodies are illustrated in FIG. 10. Unless stated otherwise, the six CDR sequences of the humanised antibodies are identical to the six CDR sequences of the parental antibody 4-H3.

4-H3 Parental (hIgG1)

In one embodiment, the humanised antibody or antigen-binding fragment thereof is referred to herein as 4-H3_parental (hIgG1).

Preferably, the antibody or antigen-binding fragment thereof comprises a heavy chain variable (VH) region comprising or consisting of a sequence as substantially set out in SEQ ID No: 159, or a variant or fragment thereof.

Preferably, the antibody or antigen-binding fragment thereof comprises a light chain variable (VL) region comprising or consisting of a sequence as substantially set out in SEQ ID No: 43, or a variant or fragment thereof.

Preferably, the antibody or antigen-binding fragment thereof comprises a heavy chain variable region comprising or consisting of SEQ ID No: 159 and a light chain variable region comprising or consisting of SEQ ID No: 43.

Preferably, the antibody or antigen-binding fragment thereof comprises a heavy chain constant (HC) region comprising or consisting of a sequence as substantially set out in

SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

4-H3 Parental (hIgG1-L234A-L235A-P329G)

Alternatively, in another embodiment, the humanised antibody or antigen-binding fragment thereof is referred to herein as 4-H3_parental (hIgG1-L234A-L235A-P329G).

Preferably, the antibody or antigen-binding fragment thereof comprises a heavy chain variable (VH) region comprising or consisting of a sequence as substantially set out in SEQ ID No: 159, or a variant or fragment thereof.

Preferably, the antibody or antigen-binding fragment thereof comprises a light chain variable (VL) region comprising or consisting of a sequence as substantially set out in SEQ ID No: 43, or a variant or fragment thereof.

Preferably, the antibody or antigen-binding fragment thereof comprises a heavy chain variable region comprising or consisting of SEQ ID No: 159 and a light chain variable region comprising or consisting of SEQ ID No: 43.

Preferably, the antibody or antigen-binding fragment thereof comprises a heavy chain constant (HC) region comprising or consisting of a sequence as substantially set out in SEQ ID No: 162, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 162 and a light chain constant region comprising or consisting of SEQ ID No: 158.

4-H3 Parental (hIgG1-Fab)

Alternatively, in another embodiment, the humanised antibody or antigen-binding fragment thereof is referred to herein as 4-H3_parental (hIgG1-Fab).

Preferably, the antibody or antigen-binding fragment thereof comprises a heavy chain variable (VH) region comprising or consisting of a sequence as substantially set out in SEQ ID No: 159, or a variant or fragment thereof.

Preferably, the antibody or antigen-binding fragment thereof comprises a light chain variable (VL) region comprising or consisting of a sequence as substantially set out in SEQ ID No: 43, or a variant or fragment thereof.

Preferably, the antibody or antigen-binding fragment thereof comprises a heavy chain variable region comprising or consisting of SEQ ID No: 159 and a light chain variable region comprising or consisting of SEQ ID No: 43.

Preferably, the antibody or antigen-binding fragment thereof comprises a heavy chain constant (HC) region comprising or consisting of a sequence as substantially set out in SEQ ID No: 163, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 163 and a light chain constant region comprising or consisting of SEQ ID No: 158.

4-H3 H0 (hIgG1)

Alternatively, in another embodiment, the humanised antibody or antigen-binding fragment thereof is referred to herein as 4-H3_H0 (hIgG1).

Preferably, the antibody or antigen-binding fragment thereof comprises a CDR-H1 domain comprising or consisting of a sequence as substantially set out in SEQ ID No: 164, or a variant or fragment thereof. Preferably, the antibody or antigen-binding fragment thereof comprises a CDR-H2 domain comprising or consisting of a sequence as substantially set out in SEQ ID No: 28, or a variant or fragment thereof. Preferably, the antibody or antigen-binding fragment thereof comprises a CDR-H3 domain comprising or consisting of a sequence as substantially set out in SEQ ID No: 29, or a variant or fragment thereof.

Preferably, the antibody or antigen-binding fragment thereof comprises a CDR-L1 domain comprising or consisting of a sequence as substantially set out in SEQ ID No: 36, or a variant or fragment thereof. Preferably, the antibody or antigen-binding fragment thereof comprises a CDR-L2 domain comprising or consisting of a sequence as substantially set out in SEQ ID No: 37, or a variant or fragment thereof. Preferably, the antibody or antigen-binding fragment thereof comprises a CDR-L3 domain comprising or consisting of a sequence as substantially set out in SEQ ID No: 38, or a variant or fragment thereof.

Preferably, the antibody or antigen-binding fragment thereof comprises a CDR-H1 domain comprising or consisting of SEQ ID No: 164, a CDR-H2 domain comprising or consisting of SEQ ID No: 28; a CDR-H3 domain comprising or consisting of SEQ ID No: 29, a CDR-L1 domain comprising or consisting of SEQ ID No: 36, a CDR-L2 domain comprising or consisting of SEQ ID No: 37, and a CDR-L3 domain comprising or consisting of SEQ ID No: 38.

Preferably, the antibody or antigen-binding fragment thereof comprises a heavy chain variable (VH) region comprising or consisting of a sequence as substantially set out in SEQ ID No: 165, or a variant or fragment thereof.

Preferably, the antibody or antigen-binding fragment thereof comprises a light chain variable (VL) region comprising or consisting of a sequence as substantially set out in SEQ ID No: 43, or a variant or fragment thereof.

Preferably, the antibody or antigen-binding fragment thereof comprises a heavy chain variable region comprising or consisting of SEQ ID No: 165 and a light chain variable region comprising or consisting of SEQ ID No: 43.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

4-H3 H1 (hIgG1)

Alternatively, in another embodiment, the humanised antibody or antigen-binding fragment thereof is referred to herein as 4-H3_H1 (hIgG1).

Preferably, the antibody or antigen-binding fragment thereof comprises a heavy chain variable (VH) region comprising or consisting of a sequence as substantially set out in SEQ ID No: 194, or a variant or fragment thereof.

Preferably, the antibody or antigen-binding fragment thereof comprises a light chain variable (VL) region comprising or consisting of a sequence as substantially set out in SEQ ID No: 43, or a variant or fragment thereof.

Preferably, the antibody or antigen-binding fragment thereof comprises a heavy chain variable region comprising or consisting of SEQ ID No: 194 and a light chain variable region comprising or consisting of SEQ ID No: 43.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

4-H3 H2 (hIgG1)

Alternatively, in another embodiment, the humanised antibody or antigen-binding fragment thereof is referred to herein as 4-H3_H2 (hIgG1).

Preferably, the antibody or antigen-binding fragment thereof comprises a heavy chain variable (VH) region comprising or consisting of a sequence as substantially set out in SEQ ID No: 166, or a variant or fragment thereof.

Preferably, the antibody or antigen-binding fragment thereof comprises a light chain variable (VL) region comprising or consisting of a sequence as substantially set out in SEQ ID No: 43, or a variant or fragment thereof.

Preferably, the antibody or antigen-binding fragment thereof comprises a heavy chain variable region comprising or consisting of SEQ ID No: 166 and a light chain variable region comprising or consisting of SEQ ID No: 43.

Preferably, the antibody or antigen-binding fragment thereof comprises a heavy chain constant (HC) region comprising or consisting of a sequence as substantially set out in

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

4-H3 H3 (hIgG1)

Alternatively, in another embodiment, the humanised antibody or antigen-binding fragment thereof is referred to herein as 4-H3_H3 (hIgG1).

Preferably, the antibody or antigen-binding fragment thereof comprises a heavy chain variable (VH) region comprising or consisting of a sequence as substantially set out in SEQ ID No: 167, or a variant or fragment thereof.

Preferably, the antibody or antigen-binding fragment thereof comprises a light chain variable (VL) region comprising or consisting of a sequence as substantially set out in SEQ ID No: 43, or a variant or fragment thereof.

Preferably, the antibody or antigen-binding fragment thereof comprises a heavy chain variable region comprising or consisting of SEQ ID No: 167 and a light chain variable region comprising or consisting of SEQ ID No: 43.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain

4-H3 H4 (hIgG1)

Alternatively, in another embodiment, the humanised antibody or antigen-binding fragment thereof is referred to herein as 4-H3_H4 (hIgG1).

Preferably, the antibody or antigen-binding fragment thereof comprises a heavy chain variable (VH) region comprising or consisting of a sequence as substantially set out in SEQ ID No: 168, or a variant or fragment thereof.

Preferably, the antibody or antigen-binding fragment thereof comprises a light chain variable (VL) region comprising or consisting of a sequence as substantially set out in SEQ ID No: 43, or a variant or fragment thereof.

Preferably, the antibody or antigen-binding fragment thereof comprises a heavy chain variable region comprising or consisting of SEQ ID No: 168 and a light chain variable region comprising or consisting of SEQ ID No: 43.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

4-H3 H5 (hIgG1)

Alternatively, in another embodiment, the humanised antibody or antigen-binding fragment thereof is referred to herein as 4-H3_H5 (hIgG1).

Preferably, the antibody or antigen-binding fragment thereof comprises a heavy chain variable (VH) region comprising or consisting of a sequence as substantially set out in SEQ ID No: 169, or a variant or fragment thereof.

Preferably, the antibody or antigen-binding fragment thereof comprises a light chain variable (VL) region comprising or consisting of a sequence as substantially set out in SEQ ID No: 43, or a variant or fragment thereof.

Preferably, the antibody or antigen-binding fragment thereof comprises a heavy chain variable region comprising or consisting of SEQ ID No: 169 and a light chain variable region comprising or consisting of SEQ ID No: 43.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

4-H3 H6 (hIgG1)

Alternatively, in another embodiment, the humanised antibody or antigen-binding fragment thereof is referred to herein as 4-H3_H6 (hIgG1).

Preferably, the antibody or antigen-binding fragment thereof comprises a heavy chain variable (VH) region comprising or consisting of a sequence as substantially set out in SEQ ID No: 170, or a variant or fragment thereof.

Preferably, the antibody or antigen-binding fragment thereof comprises a light chain variable (VL) region comprising or consisting of a sequence as substantially set out in SEQ ID No: 43, or a variant or fragment thereof.

Preferably, the antibody or antigen-binding fragment thereof comprises a heavy chain variable region comprising or consisting of SEQ ID No: 170 and a light chain variable region comprising or consisting of SEQ ID No: 43.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

4-H3 H7 (hIgG1)

Alternatively, in another embodiment, the humanised antibody or antigen-binding fragment thereof is referred to herein as 4-H3_H7 (hIgG1).

Preferably, the antibody or antigen-binding fragment thereof comprises a heavy chain variable (VH) region comprising or consisting of a sequence as substantially set out in SEQ ID No: 171, or a variant or fragment thereof.

Preferably, the antibody or antigen-binding fragment thereof comprises a light chain variable (VL) region comprising or consisting of a sequence as substantially set out in SEQ ID No: 43, or a variant or fragment thereof.

Preferably, the antibody or antigen-binding fragment thereof comprises a heavy chain variable region comprising or consisting of SEQ ID No: 171 and a light chain variable region comprising or consisting of SEQ ID No: 43.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in

Preferably, the antibody or antigen-binding fragment thereof comprises a heavy chain constant region comprising or consisting of SEQ ID No: 157 and a light chain constant region comprising or consisting of SEQ ID No: 158.

4-H3 H8 (hIgG1)

Alternatively, in another embodiment, the humanised antibody or antigen-binding fragment thereof is referred to herein as 4-H3_H8 (hIgG1).

Preferably, the antibody or antigen-binding fragment thereof comprises a heavy chain variable (VH) region comprising or consisting of a sequence as substantially set out in SEQ ID No: 172, or a variant or fragment thereof.

Preferably, the antibody or antigen-binding fragment thereof comprises a light chain variable (VL) region comprising or consisting of a sequence as substantially set out in SEQ ID No: 43, or a variant or fragment thereof.

Preferably, the antibody or antigen-binding fragment thereof comprises a heavy chain variable region comprising or consisting of SEQ ID No: 172 and a light chain variable region comprising or consisting of SEQ ID No: 43.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

4-H3 L0 (hIgG1K)

Alternatively, in another embodiment, the humanised antibody or antigen-binding fragment thereof is referred to herein as 4-H3_L0 (hIgG1K).

Preferably, the antibody or antigen-binding fragment thereof comprises a heavy chain variable (VH) region comprising or consisting of a sequence as substantially set out in SEQ ID No: 159, or a variant or fragment thereof.

Preferably, the antibody or antigen-binding fragment thereof comprises a light chain variable (VL) region comprising or consisting of a sequence as substantially set out in SEQ ID No: 185, or a variant or fragment thereof.

Preferably, the antibody or antigen-binding fragment thereof comprises a heavy chain variable region comprising or consisting of SEQ ID No: 159 and a light chain variable region comprising or consisting of SEQ ID No: 185.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

4-H3 L1 (hIgG1K)

Alternatively, in another embodiment, the humanised antibody or antigen-binding fragment thereof is referred to herein as 4-H3_L1 (hIgG1K).

Preferably, the antibody or antigen-binding fragment thereof comprises a heavy chain variable (VH) region comprising or consisting of a sequence as substantially set out in SEQ ID No: 159, or a variant or fragment thereof.

Preferably, the antibody or antigen-binding fragment thereof comprises a light chain variable (VL) region comprising or consisting of a sequence as substantially set out in SEQ ID No: 186, or a variant or fragment thereof.

Preferably, the antibody or antigen-binding fragment thereof comprises a heavy chain variable region comprising or consisting of SEQ ID No: 159 and a light chain variable region comprising or consisting of SEQ ID No: 186.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 4-H3 H8 L1

Alternatively, in another embodiment, the humanised antibody or antigen-binding fragment thereof is referred to herein as 4-H3_H8_L1.

Preferably, the antibody or antigen-binding fragment thereof comprises a heavy chain variable (VH) region comprising or consisting of a sequence as substantially set out in SEQ ID No: 172, or a variant or fragment thereof.

Preferably, the antibody or antigen-binding fragment thereof comprises a light chain variable (VL) region comprising or consisting of a sequence as substantially set out in SEQ ID No: 186, or a variant or fragment thereof.

Preferably, the antibody or antigen-binding fragment thereof comprises a heavy chain variable region comprising or consisting of SEQ ID No: 172 and a light chain variable region comprising or consisting of SEQ ID No: 186.

Preferably, the antibody or antigen-binding fragment thereof comprises a heavy chain constant (HC) region comprising or consisting of a sequence as substantially set out in

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 4-H3 H8 L0

Alternatively, in another embodiment, the humanised antibody or antigen-binding fragment thereof is referred to herein as 4-H3_H8_L0.

Preferably, the antibody or antigen-binding fragment thereof comprises a heavy chain variable (VH) region comprising or consisting of a sequence as substantially set out in SEQ ID No: 172, or a variant or fragment thereof.

Preferably, the antibody or antigen-binding fragment thereof comprises a light chain variable (VL) region comprising or consisting of a sequence as substantially set out in SEQ ID No: 185, or a variant or fragment thereof.

Preferably, the antibody or antigen-binding fragment thereof comprises a heavy chain variable region comprising or consisting of SEQ ID No: 172 and a light chain variable region comprising or consisting of SEQ ID No: 185.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain

### 4-H3 H7 L1

Alternatively, in another embodiment, the humanised antibody or antigen-binding fragment thereof is referred to herein as 4-H3_H7_L1.

Preferably, the antibody or antigen-binding fragment thereof comprises a heavy chain variable (VH) region comprising or consisting of a sequence as substantially set out in SEQ ID No: 171, or a variant or fragment thereof.

Preferably, the antibody or antigen-binding fragment thereof comprises a light chain variable (VL) region comprising or consisting of a sequence as substantially set out in SEQ ID No: 186, or a variant or fragment thereof.

Preferably, the antibody or antigen-binding fragment thereof comprises a heavy chain variable region comprising or consisting of SEQ ID No: 171 and a light chain variable region comprising or consisting of SEQ ID No: 186.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 4-H3 H7 L0 rep

Alternatively, in another embodiment, the humanised antibody or antigen-binding fragment thereof is referred to herein as 4-H3_H7_L0 rep.

Preferably, the antibody or antigen-binding fragment thereof comprises a heavy chain variable (VH) region comprising or consisting of a sequence as substantially set out in SEQ ID No: 171, or a variant or fragment thereof.

Preferably, the antibody or antigen-binding fragment thereof comprises a light chain variable (VL) region comprising or consisting of a sequence as substantially set out in SEQ ID No: 185, or a variant or fragment thereof.

Preferably, the antibody or antigen-binding fragment thereof comprises a heavy chain variable region comprising or consisting of SEQ ID No: 171 and a light chain variable region comprising or consisting of SEQ ID No: 185.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 4-H3 H6 L1

Alternatively, in another embodiment, the humanised antibody or antigen-binding fragment thereof is referred to herein as 4-H3_H6_L1.

Preferably, the antibody or antigen-binding fragment thereof comprises a heavy chain variable (VH) region comprising or consisting of a sequence as substantially set out in SEQ ID No: 170, or a variant or fragment thereof.

Preferably, the antibody or antigen-binding fragment thereof comprises a light chain variable (VL) region comprising or consisting of a sequence as substantially set out in SEQ ID No: 186, or a variant or fragment thereof.

Preferably, the antibody or antigen-binding fragment thereof comprises a heavy chain variable region comprising or consisting of SEQ ID No: 170 and a light chain variable region comprising or consisting of SEQ ID No: 186.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 4-H3 H6 L0

Alternatively, in another embodiment, the humanised antibody or antigen-binding fragment thereof is referred to herein as 4-H3_H6_L0.

Preferably, the antibody or antigen-binding fragment thereof comprises a heavy chain variable (VH) region comprising or consisting of a sequence as substantially set out in SEQ ID No: 170, or a variant or fragment thereof.

Preferably, the antibody or antigen-binding fragment thereof comprises a light chain variable (VL) region comprising or consisting of a sequence as substantially set out in SEQ ID No: 185, or a variant or fragment thereof.

Preferably, the antibody or antigen-binding fragment thereof comprises a heavy chain variable region comprising or consisting of SEQ ID No: 170 and a light chain variable region comprising or consisting of SEQ ID No: 185.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in

Preferably, the antibody or antigen-binding fragment thereof comprises a heavy chain constant region comprising or consisting of SEQ ID No: 157 and a light chain constant region comprising or consisting of SEQ ID No: 158.

### 4-H3 H5 L1

Alternatively, in another embodiment, the humanised antibody or antigen-binding fragment thereof is referred to herein as 4-H3_H5_L1.

Preferably, the antibody or antigen-binding fragment thereof comprises a heavy chain variable (VH) region comprising or consisting of a sequence as substantially set out in SEQ ID No: 169, or a variant or fragment thereof.

Preferably, the antibody or antigen-binding fragment thereof comprises a light chain variable (VL) region comprising or consisting of a sequence as substantially set out in SEQ ID No: 186, or a variant or fragment thereof.

Preferably, the antibody or antigen-binding fragment thereof comprises a heavy chain variable region comprising or consisting of SEQ ID No: 169 and a light chain variable region comprising or consisting of SEQ ID No: 186.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 4-H3 H5 L0

Alternatively, in another embodiment, the humanised antibody or antigen-binding fragment thereof is referred to herein as 4-H3_H5_L0.

Preferably, the antibody or antigen-binding fragment thereof comprises a heavy chain variable (VH) region comprising or consisting of a sequence as substantially set out in SEQ ID No: 169, or a variant or fragment thereof.

Preferably, the antibody or antigen-binding fragment thereof comprises a light chain variable (VL) region comprising or consisting of a sequence as substantially set out in SEQ ID No: 185, or a variant or fragment thereof.

Preferably, the antibody or antigen-binding fragment thereof comprises a heavy chain variable region comprising or consisting of SEQ ID No: 169 and a light chain variable region comprising or consisting of SEQ ID No: 185.

Preferably, the antibody or antigen-binding fragment thereof comprises a heavy chain constant (HC) region comprising or consisting of a sequence as substantially set out in

SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 4-H3 H4 L1

Alternatively, in another embodiment, the humanised antibody or antigen-binding fragment thereof is referred to herein as 4-H3_H4_L1.

Preferably, the antibody or antigen-binding fragment thereof comprises a heavy chain variable (VH) region comprising or consisting of a sequence as substantially set out in SEQ ID No: 168, or a variant or fragment thereof.

Preferably, the antibody or antigen-binding fragment thereof comprises a light chain variable (VL) region comprising or consisting of a sequence as substantially set out in SEQ ID No: 186, or a variant or fragment thereof.

Preferably, the antibody or antigen-binding fragment thereof comprises a heavy chain variable region comprising or consisting of SEQ ID No: 168 and a light chain variable region comprising or consisting of SEQ ID No: 186.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 4-H3 H4 L0

Alternatively, in another embodiment, the humanised antibody or antigen-binding fragment thereof is referred to herein as 4-H3_H4_L0.

Preferably, the antibody or antigen-binding fragment thereof comprises a heavy chain variable (VH) region comprising or consisting of a sequence as substantially set out in SEQ ID No: 168, or a variant or fragment thereof.

Preferably, the antibody or antigen-binding fragment thereof comprises a light chain variable (VL) region comprising or consisting of a sequence as substantially set out in SEQ ID No: 185, or a variant or fragment thereof.

Preferably, the antibody or antigen-binding fragment thereof comprises a heavy chain variable region comprising or consisting of SEQ ID No: 168 and a light chain variable region comprising or consisting of SEQ ID No: 185.

Preferably, the antibody or antigen-binding fragment thereof comprises a heavy chain constant (HC) region comprising or consisting of a sequence as substantially set out in

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 4-H3 H3 L1

Alternatively, in another embodiment, the humanised antibody or antigen-binding fragment thereof is referred to herein as 4-H3_H3_L1.

Preferably, the antibody or antigen-binding fragment thereof comprises a heavy chain variable (VH) region comprising or consisting of a sequence as substantially set out in

SEQ ID No: 167, or a variant or fragment thereof.

Preferably, the antibody or antigen-binding fragment thereof comprises a light chain variable (VL) region comprising or consisting of a sequence as substantially set out in SEQ ID No: 186, or a variant or fragment thereof.

Preferably, the antibody or antigen-binding fragment thereof comprises a heavy chain variable region comprising or consisting of SEQ ID No: 167 and a light chain variable region comprising or consisting of SEQ ID No: 186.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain

### 4-H3 H3 L0

Alternatively, in another embodiment, the humanised antibody or antigen-binding fragment thereof is referred to herein as 4-H3_H3_L0.

Preferably, the antibody or antigen-binding fragment thereof comprises a heavy chain variable (VH) region comprising or consisting of a sequence as substantially set out in SEQ ID No: 167, or a variant or fragment thereof.

Preferably, the antibody or antigen-binding fragment thereof comprises a light chain variable (VL) region comprising or consisting of a sequence as substantially set out in SEQ ID No: 185, or a variant or fragment thereof.

Preferably, the antibody or antigen-binding fragment thereof comprises a heavy chain variable region comprising or consisting of SEQ ID No: 167 and a light chain variable region comprising or consisting of SEQ ID No: 185.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 4-H3 H2 L1

Alternatively, in another embodiment, the humanised antibody or antigen-binding fragment thereof is referred to herein as 4-H3_H2_L1.

Preferably, the antibody or antigen-binding fragment thereof comprises a heavy chain variable (VH) region comprising or consisting of a sequence as substantially set out in SEQ ID No: 166, or a variant or fragment thereof.

Preferably, the antibody or antigen-binding fragment thereof comprises a light chain variable (VL) region comprising or consisting of a sequence as substantially set out in SEQ ID No: 186, or a variant or fragment thereof.

Preferably, the antibody or antigen-binding fragment thereof comprises a heavy chain variable region comprising or consisting of SEQ ID No: 166 and a light chain variable region comprising or consisting of SEQ ID No: 186.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 4-H3 H2 L0

Alternatively, in another embodiment, the humanised antibody or antigen-binding fragment thereof is referred to herein as 4-H3_H2_L0.

Preferably, the antibody or antigen-binding fragment thereof comprises a heavy chain variable (VH) region comprising or consisting of a sequence as substantially set out in SEQ ID No: 166, or a variant or fragment thereof.

Preferably, the antibody or antigen-binding fragment thereof comprises a light chain variable (VL) region comprising or consisting of a sequence as substantially set out in SEQ ID No: 185, or a variant or fragment thereof.

Preferably, the antibody or antigen-binding fragment thereof comprises a heavy chain variable region comprising or consisting of SEQ ID No: 166 and a light chain variable region comprising or consisting of SEQ ID No: 185.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 4-H3 H1 L1

Alternatively, in another embodiment, the humanised antibody or antigen-binding fragment thereof is referred to herein as 4-H3_H1_L1.

Preferably, the antibody or antigen-binding fragment thereof comprises a heavy chain variable (VH) region comprising or consisting of a sequence as substantially set out in SEQ ID No: 194, or a variant or fragment thereof.

Preferably, the antibody or antigen-binding fragment thereof comprises a light chain variable (VL) region comprising or consisting of a sequence as substantially set out in SEQ ID No: 186, or a variant or fragment thereof.

Preferably, the antibody or antigen-binding fragment thereof comprises a heavy chain variable region comprising or consisting of SEQ ID No: 194 and a light chain variable region comprising or consisting of SEQ ID No: 186.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in

Preferably, the antibody or antigen-binding fragment thereof comprises a heavy chain constant region comprising or consisting of SEQ ID No: 157 and a light chain constant region comprising or consisting of SEQ ID No: 158.

### 4-H3 H1 L0

Alternatively, in another embodiment, the humanised antibody or antigen-binding fragment thereof is referred to herein as 4-H3_H1_L0.

Preferably, the antibody or antigen-binding fragment thereof comprises a heavy chain variable (VH) region comprising or consisting of a sequence as substantially set out in SEQ ID No: 194, or a variant or fragment thereof.

Preferably, the antibody or antigen-binding fragment thereof comprises a light chain variable (VL) region comprising or consisting of a sequence as substantially set out in

SEQ ID No: 185, or a variant or fragment thereof.

Preferably, the antibody or antigen-binding fragment thereof comprises a heavy chain variable region comprising or consisting of SEQ ID No: 194 and a light chain variable region comprising or consisting of SEQ ID No: 185.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 4-H3 H0 L1 rep

Alternatively, in another embodiment, the humanised antibody or antigen-binding fragment thereof is referred to herein as 4-H3_H0_L1 rep.

Preferably, the antibody or antigen-binding fragment thereof comprises a CDR-H1 domain comprising or consisting of a sequence as substantially set out in SEQ ID No: 164, or a variant or fragment thereof. Preferably, the antibody or antigen-binding fragment thereof comprises a CDR-H2 domain comprising or consisting of a sequence as substantially set out in SEQ ID No: 28, or a variant or fragment thereof. Preferably, the antibody or antigen-binding fragment thereof comprises a CDR-H3 domain comprising or consisting of a sequence as substantially set out in SEQ ID No: 29, or a variant or fragment thereof.

Preferably, the antibody or antigen-binding fragment thereof comprises a CDR-L1 domain comprising or consisting of a sequence as substantially set out in SEQ ID No: 36, or a variant or fragment thereof. Preferably, the antibody or antigen-binding fragment thereof comprises a CDR-L2 domain comprising or consisting of a sequence as substantially set out in SEQ ID No: 37, or a variant or fragment thereof. Preferably, the antibody or antigen-binding fragment thereof comprises a CDR-L3 domain comprising or consisting of a sequence as substantially set out in SEQ ID No: 38, or a variant or fragment thereof.

Preferably, the antibody or antigen-binding fragment thereof comprises a CDR-H1 domain comprising or consisting of SEQ ID No: 164, a CDR-H2 domain comprising or consisting of SEQ ID No: 28; a CDR-H3 domain comprising or consisting of SEQ ID No: 29, a CDR-L1 domain comprising or consisting of SEQ ID No: 36, a CDR-L2 domain comprising or consisting of SEQ ID No: 37, and a CDR-L3 domain comprising or consisting of SEQ ID No: 38.

Preferably, the antibody or antigen-binding fragment thereof comprises a heavy chain variable (VH) region comprising or consisting of a sequence as substantially set out in SEQ ID No: 165, or a variant or fragment thereof.

Preferably, the antibody or antigen-binding fragment thereof comprises a light chain variable (VL) region comprising or consisting of a sequence as substantially set out in SEQ ID No: 186, or a variant or fragment thereof.

Preferably, the antibody or antigen-binding fragment thereof comprises a heavy chain variable region comprising or consisting of SEQ ID No: 165 and a light chain variable region comprising or consisting of SEQ ID No: 186.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 4-H3 H0 L0

Alternatively, in another embodiment, the humanised antibody or antigen-binding fragment thereof is referred to herein as 4-H3_H0_L0.

Preferably, the antibody or antigen-binding fragment thereof comprises a CDR-H1 domain comprising or consisting of a sequence as substantially set out in SEQ ID No: 164, or a variant or fragment thereof. Preferably, the antibody or antigen-binding fragment thereof comprises a CDR-H2 domain comprising or consisting of a sequence as substantially set out in SEQ ID No: 28, or a variant or fragment thereof. Preferably, the antibody or antigen-binding fragment thereof comprises a CDR-H3 domain comprising or consisting of a sequence as substantially set out in SEQ ID No: 29, or a variant or fragment thereof.

Preferably, the antibody or antigen-binding fragment thereof comprises a CDR-L1 domain comprising or consisting of a sequence as substantially set out in SEQ ID No: 36, or a variant or fragment thereof. Preferably, the antibody or antigen-binding fragment thereof comprises a CDR-L2 domain comprising or consisting of a sequence as substantially set out in SEQ ID No: 37, or a variant or fragment thereof. Preferably, the antibody or antigen-binding fragment thereof comprises a CDR-L3 domain comprising or consisting of a sequence as substantially set out in SEQ ID No: 38, or a variant or fragment thereof.

Preferably, the antibody or antigen-binding fragment thereof comprises a CDR-H1 domain comprising or consisting of SEQ ID No: 164, a CDR-H2 domain comprising or consisting of SEQ ID No: 28; a CDR-H3 domain comprising or consisting of SEQ ID No: 29, a CDR-L1 domain comprising or consisting of SEQ ID No: 36, a CDR-L2 domain comprising or consisting of SEQ ID No: 37, and a CDR-L3 domain comprising or consisting of SEQ ID No: 38.

Preferably, the antibody or antigen-binding fragment thereof comprises a heavy chain variable (VH) region comprising or consisting of a sequence as substantially set out in SEQ ID No: 165, or a variant or fragment thereof.

Preferably, the antibody or antigen-binding fragment thereof comprises a light chain variable (VL) region comprising or consisting of a sequence as substantially set out in SEQ ID No: 185, or a variant or fragment thereof.

Preferably, the antibody or antigen-binding fragment thereof comprises a heavy chain variable region comprising or consisting of SEQ ID No: 165 and a light chain variable region comprising or consisting of SEQ ID No: 185.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-D5

In one embodiment, the antibody or antigen-binding fragment thereof is referred to herein as 1-D5. The antibody or antigen-binding fragment thereof may comprise a CDR-H1 domain of SEQ ID No: 45, which is provided herein, as follows:

[SEQ ID No: 45]
GFSLNNYI

Thus, preferably, the antibody or antigen-binding fragment thereof comprises a CDR-H1 domain comprising or consisting of a sequence as substantially set out in SEQ ID No: 45, or a variant or fragment thereof.

The antibody or antigen-binding fragment thereof may comprise a CDR-H2 domain of SEQ ID No: 46, which is provided herein, as follows:

[SEQ ID No: 46]
ISTGGST

Thus, preferably the antibody or antigen-binding fragment thereof comprises a CDR-H2 domain comprising or consisting of a sequence as substantially set out in SEQ ID No: 46, or a variant or fragment thereof.

The antibody or antigen-binding fragment thereof may comprise a CDR-H3 domain of SEQ ID No: 47, which is provided herein, as follows:

[SEQ ID No: 47]
ARGGSSAGAGENI

Thus, preferably the antibody or antigen-binding fragment thereof comprises a CDR-H3 domain comprising or consisting of a sequence as substantially set out in SEQ ID No: 47, or a variant or fragment thereof.

Preferably, the antibody or antigen-binding fragment thereof comprises a CDR-H1 domain comprising or consisting of SEQ ID No: 45, a CDR-H2 domain comprising or consisting of SEQ ID No: 46 and/or a CDR-H3 domain comprising or consisting of SEQ ID No: 47. Preferably, however, the antibody or antigen-binding fragment thereof comprises a CDR-H1 domain comprising or consisting of SEQ ID No: 45, a CDR-H2 domain comprising or consisting of SEQ ID No: 46 and a CDR-H3 domain comprising or consisting of SEQ ID No: 47.

The antibody or antigen-binding fragment thereof may comprise a FR-H1 domain of SEQ ID No: 48, which is provided herein, as follows:

[SEQ ID No: 48]
QQQLVESGGRLVTPGTPLTLTCAVS

Thus, preferably, the antibody or antigen-binding fragment thereof comprises a FR-H1 domain comprising or consisting of a sequence as substantially set out in SEQ ID No: 48, or a variant or fragment thereof.

The antibody or antigen-binding fragment thereof may comprise a FR-H2 domain of SEQ ID No: 49, which is provided herein, as follows:

[SEQ ID No: 49]
MGWVRQAPGKGLEYIGI

Thus, preferably, the antibody or antigen-binding fragment thereof comprises a FR-H2 domain comprising or consisting of a sequence as substantially set out in SEQ ID No: 49, or a variant or fragment thereof.

The antibody or antigen-binding fragment thereof may comprise a FR-H3 domain of SEQ ID No: 50, which is provided herein, as follows:

[SEQ ID No: 50]
YYASWAKGRFTISRTSTTMDLKMTSLTTEDTATYFC

Thus, preferably, the antibody or antigen-binding fragment thereof comprises a FR-H3 domain comprising or consisting of a sequence as substantially set out in SEQ ID No: 50, or a variant or fragment thereof.

The antibody or antigen-binding fragment thereof may comprise a FR-H4 domain of SEQ ID No: 51, which is provided herein, as follows:

[SEQ ID No: 51]
WGPGTLVTVSS

Thus, preferably, the antibody or antigen-binding fragment thereof comprises a FR-H4 domain comprising or consisting of a sequence as substantially set out in SEQ ID No: 51, or a variant or fragment thereof.

Preferably, the antibody or antigen-binding fragment thereof comprises a FR-H1 domain comprising or consisting of SEQ ID No: 48, a FR-H2 domain comprising or consisting of SEQ ID No: 49, a FR-H3 domain comprising or consisting of SEQ ID No: 50, and/or a FR-H4 domain comprising or consisting of SEQ ID No: 51. Preferably, however, the antibody or antigen-binding fragment thereof comprises a FR-H1 domain comprising or consisting of SEQ ID No: 48, a FR-H2 domain comprising or consisting of SEQ ID No: 49, a FR-H3 domain comprising or consisting of SEQ ID No: 50, and a FR-H4 domain comprising or consisting of SEQ ID No: 51.

The antibody or antigen-binding fragment thereof may comprise a heavy chain variable (VH) sequence as set out in SEQ ID No: 52, which is provided herein, as follows:

[SEQ ID No: 52]
QQQLVESGGRLVTPGTPLTLTCAVSGFSLNNYIMGWVRQAPGKGLEYIG
IISTGGSTYYASWAKGRFTISRTSTTMDLKMTSLTTEDTATYFCARGGS
SAGAGFNIWGPGTLVTVSS

Preferably, the antibody or antigen-binding fragment thereof comprises a heavy chain variable (VH) region comprising or consisting of a sequence as substantially set out in SEQ ID No: 52, or a variant or fragment thereof.

One embodiment of the nucleotide sequence encoding the heavy chain variable (VH) region is referred to herein as SEQ ID No: 53, as follows:

[SEQ ID No: 53]
CAGCAGCAGCTGGTGGAGTCCGGGGGTCGCCTGGTCACGCCTGGGACAC
CCCTGACACTAACCTGCGCAGTCTCTGGATTTTCCCTCAATAACTACAT
CATGGGCTGGGTCCGCCAGGCTCCAGGGAAGGGGCTGGAATACATCGGA
ATCATTAGTACTGGTGGTAGCACATACTACGCGAGCTGGGCAAAAGGCC
GATTCACCATCTCCAGAACCTCGACCACGATGGATCTGAAAATGACCAG
TCTGACAACCGAGGACACGGCCACCTATTTCTGTGCCAGAGGGGGTAGT
AGTGCTGGTGCGGGATTTAATATCTGGGGCCCGGGCACCCTGGTCACCG
TCTCCTCA

Preferably, the antibody or antigen-binding fragment thereof comprises a heavy chain variable (VH) region encoded by a nucleic acid sequence as substantially set out in SEQ ID No: 53, or a variant or fragment thereof.

The antibody or antigen-binding fragment thereof may comprise a CDR-L1 domain of SEQ ID No: 54, which is provided herein, as follows:

[SEQ ID No: 54]
QSINSG

Thus, preferably, the antibody or antigen-binding fragment thereof comprises a CDR-L1 domain comprising or consisting of a sequence as substantially set out in SEQ ID No: 54, or a variant or fragment thereof.

The antibody or antigen-binding fragment thereof may comprise a CDR-L2 domain of SEQ ID No: 55, which is provided herein, as follows:

[SEQ ID No: 55]
KAS

Thus, preferably, the antibody or antigen-binding fragment thereof comprises a CDR-L2 domain comprising or consisting of a sequence as substantially set out in SEQ ID No: 55, or a variant or fragment thereof.

The antibody or antigen-binding fragment thereof may comprise a CDR-L3 domain of SEQ ID No: 56, which is provided herein, as follows:

[SEQ ID No: 56]
QSYHYISANGAT

Thus, preferably, the antibody or antigen-binding fragment thereof comprises a CDR-L3 domain comprising or consisting of a sequence as substantially set out in SEQ ID No: 56, or a variant or fragment thereof.

Preferably, the antibody or antigen-binding fragment thereof comprises a CDR-L1 domain comprising or consisting of SEQ ID No: 54, a CDR-L2 domain comprising or consisting of SEQ ID No: 55, and/or a CDR-L3 domain comprising or consisting of SEQ ID No: 56. However, preferably the antibody or antigen-binding fragment thereof comprises a CDR-L1 domain comprising or consisting of SEQ ID No: 54, a CDR-L2 domain comprising or consisting of SEQ ID No: 55, and a CDR-L3 domain comprising or consisting of SEQ ID No: 56.

The antibody or antigen-binding fragment thereof may comprise a FR-L1 domain of SEQ ID No: 57, which is provided herein, as follows:

[SEQ ID No: 57]
DIVMTQTPSSVSAAVGDTVTIQCQAS

Thus, preferably, the antibody or antigen-binding fragment thereof comprises a FR-L1 domain comprising or consisting of a sequence as substantially set out in SEQ ID No: 57, or a variant or fragment thereof.

The antibody or antigen-binding fragment thereof may comprise a FR-L2 domain of SEQ ID No: 58, which is provided herein, as follows:

[SEQ ID No: 58]
LAWYQQKPGQPPKRLIY

Thus, preferably, the antibody or antigen-binding fragment thereof comprises a FR-L2 domain comprising or consisting of a sequence as substantially set out in SEQ ID No: 58, or a variant or fragment thereof.

The antibody or antigen-binding fragment thereof may comprise a FR-L3 domain of SEQ ID No: 59, which is provided herein, as follows:

[SEQ ID No: 59]
TLASGVPSRFRGSGSGTDFTLTISDLECADAATYYC

Thus, preferably, the antibody or antigen-binding fragment thereof comprises a FR-L3 domain comprising or consisting of a sequence as substantially set out in SEQ ID No: 59, or a variant or fragment thereof.

The antibody or antigen-binding fragment thereof may comprise a FR-L4 domain of SEQ ID No: 60, which is provided herein, as follows:

[SEQ ID No: 60]
FGGGTEVVVE

Thus, preferably, the antibody or antigen-binding fragment thereof comprises a FR-L4 domain comprising or consisting of a sequence as substantially set out in SEQ ID No: 60, or a variant or fragment thereof.

Preferably, the antibody or antigen-binding fragment thereof comprises a FR-L1 domain comprising or consisting of SEQ ID No: 57, a FR-L2 domain comprising or consisting of SEQ ID No: 58, a FR-L3 domain comprising or consisting of SEQ ID No: 59, and/or a FR-L4 domain comprising or consisting of SEQ ID No: 60. Preferably, however, the antibody or antigen-binding fragment thereof comprises a FR-L1 domain comprising or consisting of SEQ ID No: 57, a FR-L2 domain comprising or consisting of SEQ ID No: 58, a FR-L3 domain comprising or consisting of SEQ ID No: 59, and a FR-L4 domain comprising or consisting of SEQ ID No: 60.

The antibody or antigen-binding fragment thereof may comprise a light chain variable (VL) sequence as set out in SEQ ID No: 61, which is provided herein, as follows:

[SEQ ID No: 61]
DIVMTQTPSSVSAAVGDTVTIQCQASQSINSGLAWYQQKPGQPPKRLIY
KASTLASGVPSRFRGSGSGTDFTLTISDLECADAATYYCQSYHYISANG
ATFGGGTEVVVE

Preferably, the antibody or antigen-binding fragment thereof comprises a light chain variable region comprising or consisting of a sequence as substantially set out in SEQ ID No: 61, or a variant or fragment thereof.

One embodiment of the nucleotide sequence encoding the light chain variable (VL) region is referred to herein as SEQ ID No: 62, as follows:

[SEQ ID No: 62]
GATATTGTTATGACCCAGACTCCCTCCTCCGTGTCTGCAGCTGTGGGAG
ACACAGTCACCATCCAGTGCCAGGCCAGTCAGAGCATTAATAGTGGTTT
GGCCTGGTATCAGCAGAAACCAGGGCAGCCTCCCAAGCGCCTGATCTAC
AAGGCATCCACTCTGGCATCTGGGGTCCCATCGCGGTTCAGAGGCAGTG
GATCTGGGACAGACTTCACTCTCACCATCAGCGACCTGGAGTGTGCCGA
TGCTGCCACTTACTACTGTCAAAGCTATCATTATATTAGTGCTAATGGT
GCTACTTTCGGCGGAGGGACCGAGGTGGTCGTCGAA

Preferably, the antibody or antigen-binding fragment thereof comprises a light chain variable (VL) region encoded by a nucleic acid sequence as substantially set out in SEQ ID No: 62, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 45, a CDR-H2 domain comprising or consisting of SEQ ID No: 46; a CDR-H3 domain comprising or consisting of SEQ ID No: 47, a CDR-L1 domain comprising or consisting of SEQ ID No: 54, a CDR-L2 domain comprising or consisting of SEQ ID No: 55, and a CDR-L3 domain comprising or consisting of SEQ ID No: 56.

Preferably, the antibody or antigen-binding fragment thereof comprises a heavy chain variable region comprising or consisting of SEQ ID No: 52, and a light chain variable region comprising or consisting of SEQ ID No: 61.

Preferably, the antibody or antigen-binding fragment thereof comprises a heavy chain variable region encoded by a nucleic acid sequence comprising or consisting of SEQ ID No: 53, and a light chain variable region encoded by a nucleic acid sequence comprising or consisting of SEQ ID No: 62.

The inventors then set out to generate humanised antibodies of 1-D5, and the sequences of the humanised antibodies are illustrated in FIG. 10. Unless stated otherwise, the six CDR sequences of the humanised antibodies are identical to the six CDR sequences of the parental antibody 1-D5.

1-D5 Parental (hIgG1)

In one embodiment, the humanised antibody or antigen-binding fragment thereof is referred to herein as 1-D5_parental (hIgG1).

Preferably, the antibody or antigen-binding fragment thereof comprises a heavy chain variable (VH) region comprising or consisting of a sequence as substantially set out in SEQ ID No: 52, or a variant or fragment thereof.

Preferably, the antibody or antigen-binding fragment thereof comprises a light chain variable (VL) region comprising or consisting of a sequence as substantially set out in SEQ ID No: 61, or a variant or fragment thereof.

Preferably, the antibody or antigen-binding fragment thereof comprises a heavy chain variable region comprising or consisting of SEQ ID No: 52 and a light chain variable region comprising or consisting of SEQ ID No: 61.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

1-D5 Parental (hIgG1-L234A-L235A-P329G)

Alternatively, in another embodiment, the humanised antibody or antigen-binding fragment thereof is referred to herein as 1-D5_parental (hIgG1-L234A-L235A-P329G).

Preferably, the antibody or antigen-binding fragment thereof comprises a heavy chain variable (VH) region comprising or consisting of a sequence as substantially set out in SEQ ID No: 52, or a variant or fragment thereof.

Preferably, the antibody or antigen-binding fragment thereof comprises a light chain variable (VL) region comprising or consisting of a sequence as substantially set out in SEQ ID No: 61, or a variant or fragment thereof.

Preferably, the antibody or antigen-binding fragment thereof comprises a heavy chain variable region comprising or consisting of SEQ ID No: 52 and a light chain variable region comprising or consisting of SEQ ID No: 61.

Preferably, the antibody or antigen-binding fragment thereof comprises a heavy chain constant (HC) region comprising or consisting of a sequence as substantially set out in SEQ ID No: 162, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 162 and a light chain constant region comprising or consisting of SEQ ID No: 158.

1-D5 Parental (hIgG1-Fab)

Alternatively, in another embodiment, the humanised antibody or antigen-binding fragment thereof is referred to herein as 1-D5_parental (hIgG1-Fab).

Preferably, the antibody or antigen-binding fragment thereof comprises a heavy chain variable (VH) region comprising or consisting of a sequence as substantially set out in

SEQ ID No: 52, or a variant or fragment thereof.

Preferably, the antibody or antigen-binding fragment thereof comprises a light chain variable (VL) region comprising or consisting of a sequence as substantially set out in SEQ ID No: 61, or a variant or fragment thereof.

Preferably, the antibody or antigen-binding fragment thereof comprises a heavy chain variable region comprising or consisting of SEQ ID No: 52 and a light chain variable region comprising or consisting of SEQ ID No: 61.

Preferably, the antibody or antigen-binding fragment thereof comprises a heavy chain constant (HC) region comprising or consisting of a sequence as substantially set out in SEQ ID No: 163, or a variant or fragment thereof.

Preferably, the antibody or antigen-binding fragment thereof comprises a light chain constant (LC) region comprising or consisting of a sequence as substantially set out in

Preferably, the antibody or antigen-binding fragment thereof comprises a heavy chain constant region comprising or consisting of SEQ ID No: 163 and a light chain constant region comprising or consisting of SEQ ID No: 158.

1-D5 H0 (hIgG1)

Alternatively, in another embodiment, the humanised antibody or antigen-binding fragment thereof is referred to herein as 1-D5_H0 (hIgG1).

Preferably, the antibody or antigen-binding fragment thereof comprises a CDR-H1 domain comprising or consisting of a sequence as substantially set out in SEQ ID No: 173, or a variant or fragment thereof. Preferably, the antibody or antigen-binding fragment thereof comprises a CDR-H2 domain comprising or consisting of a sequence as substantially set out in SEQ ID No: 46, or a variant or fragment thereof. Preferably, the antibody or antigen-binding fragment thereof comprises a CDR-H3 domain comprising or consisting of a sequence as substantially set out in SEQ ID No: 47, or a variant or fragment thereof.

Preferably, the antibody or antigen-binding fragment thereof comprises a CDR-L1 domain comprising or consisting of a sequence as substantially set out in SEQ ID No: 54, or a variant or fragment thereof. Preferably, the antibody or antigen-binding fragment thereof comprises a CDR-L2 domain comprising or consisting of a sequence as substantially set out in SEQ ID No: 55, or a variant or fragment thereof. Preferably, the antibody or antigen-binding fragment thereof comprises a CDR-L3 domain comprising or consisting of a sequence as substantially set out in SEQ ID No: 56, or a variant or fragment thereof.

Preferably, the antibody or antigen-binding fragment thereof comprises a CDR-H1 domain comprising or consisting of SEQ ID No: 173, a CDR-H2 domain comprising or consisting of SEQ ID No: 46; a CDR-H3 domain comprising or consisting of SEQ ID No: 47, a CDR-L1 domain comprising or consisting of SEQ ID No: 54, a CDR-L2 domain comprising or consisting of SEQ ID No: 55, and a CDR-L3 domain comprising or consisting of SEQ ID No: 56.

Preferably, the antibody or antigen-binding fragment thereof comprises a heavy chain variable (VH) region comprising or consisting of a sequence as substantially set out in SEQ ID No: 174, or a variant or fragment thereof.

Preferably, the antibody or antigen-binding fragment thereof comprises a light chain variable (VL) region comprising or consisting of a sequence as substantially set out in SEQ ID No: 61, or a variant or fragment thereof.

Preferably, the antibody or antigen-binding fragment thereof comprises a heavy chain variable region comprising or consisting of SEQ ID No: 174 and a light chain variable region comprising or consisting of SEQ ID No: 61.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

1-D5 H1 (hIgG1)

Alternatively, in another embodiment, the humanised antibody or antigen-binding fragment thereof is referred to herein as 1-D5_H1 (hIgG1).

Preferably, the antibody or antigen-binding fragment thereof comprises a heavy chain variable (VH) region comprising or consisting of a sequence as substantially set out in SEQ ID No: 175, or a variant or fragment thereof.

Preferably, the antibody or antigen-binding fragment thereof comprises a light chain variable (VL) region comprising or consisting of a sequence as substantially set out in SEQ ID No: 61, or a variant or fragment thereof.

Preferably, the antibody or antigen-binding fragment thereof comprises a heavy chain variable region comprising or consisting of SEQ ID No: 175 and a light chain variable region comprising or consisting of SEQ ID No: 61.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

1-D5 H2 (hIgG1)

Alternatively, in another embodiment, the humanised antibody or antigen-binding fragment thereof is referred to herein as 1-D5_H2 (hIgG1).

Preferably, the antibody or antigen-binding fragment thereof comprises a heavy chain variable (VH) region comprising or consisting of a sequence as substantially set out in SEQ ID No: 176, or a variant or fragment thereof.

Preferably, the antibody or antigen-binding fragment thereof comprises a light chain variable (VL) region comprising or consisting of a sequence as substantially set out in SEQ ID No: 61, or a variant or fragment thereof.

Preferably, the antibody or antigen-binding fragment thereof comprises a heavy chain variable region comprising or consisting of SEQ ID No: 176 and a light chain variable region comprising or consisting of SEQ ID No: 61.

Preferably, the antibody or antigen-binding fragment thereof comprises a heavy chain constant (HC) region comprising or consisting of a sequence as substantially set out in

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

1-D5 L0 (hIgG1K)

Alternatively, in another embodiment, the humanised antibody or antigen-binding fragment thereof is referred to herein as 1-D5_L0 (hIgG1K).

Preferably, the antibody or antigen-binding fragment thereof comprises a heavy chain variable (VH) region comprising or consisting of a sequence as substantially set out in SEQ ID No: 52, or a variant or fragment thereof.

Preferably, the antibody or antigen-binding fragment thereof comprises a light chain variable (VL) region comprising or consisting of a sequence as substantially set out in SEQ ID No: 187, or a variant or fragment thereof.

Preferably, the antibody or antigen-binding fragment thereof comprises a heavy chain variable region comprising or consisting of SEQ ID No: 52 and a light chain variable region comprising or consisting of SEQ ID No: 187.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain

1-D5 L1 (hIgG1K)

Alternatively, in another embodiment, the humanised antibody or antigen-binding fragment thereof is referred to herein as 1-D5_L1 (hIgG1K).

Preferably, the antibody or antigen-binding fragment thereof comprises a heavy chain variable (VH) region comprising or consisting of a sequence as substantially set out in SEQ ID No: 52, or a variant or fragment thereof.

Preferably, the antibody or antigen-binding fragment thereof comprises a light chain variable (VL) region comprising or consisting of a sequence as substantially set out in SEQ ID No: 188, or a variant or fragment thereof.

Preferably, the antibody or antigen-binding fragment thereof comprises a heavy chain variable region comprising or consisting of SEQ ID No: 52 and a light chain variable region comprising or consisting of SEQ ID No: 188.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-D5 H2 L1

Alternatively, in another embodiment, the humanised antibody or antigen-binding fragment thereof is referred to herein as 1-D5_H2_L1.

Preferably, the antibody or antigen-binding fragment thereof comprises a heavy chain variable (VH) region comprising or consisting of a sequence as substantially set out in SEQ ID No: 176, or a variant or fragment thereof.

Preferably, the antibody or antigen-binding fragment thereof comprises a light chain variable (VL) region comprising or consisting of a sequence as substantially set out in SEQ ID No: 188, or a variant or fragment thereof.

Preferably, the antibody or antigen-binding fragment thereof comprises a heavy chain variable region comprising or consisting of SEQ ID No: 176 and a light chain variable region comprising or consisting of SEQ ID No: 188.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-D5 H2 L0

Alternatively, in another embodiment, the humanised antibody or antigen-binding fragment thereof is referred to herein as 1-D5_H2_L0.

Preferably, the antibody or antigen-binding fragment thereof comprises a heavy chain variable (VH) region comprising or consisting of a sequence as substantially set out in SEQ ID No: 176, or a variant or fragment thereof.

Preferably, the antibody or antigen-binding fragment thereof comprises a light chain variable (VL) region comprising or consisting of a sequence as substantially set out in SEQ ID No: 187, or a variant or fragment thereof.

Preferably, the antibody or antigen-binding fragment thereof comprises a heavy chain variable region comprising or consisting of SEQ ID No: 176 and a light chain variable region comprising or consisting of SEQ ID No: 187.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-D5 H1 L1

Alternatively, in another embodiment, the humanised antibody or antigen-binding fragment thereof is referred to herein as 1-D5_H1_L1.

Preferably, the antibody or antigen-binding fragment thereof comprises a heavy chain variable (VH) region comprising or consisting of a sequence as substantially set out in SEQ ID No: 175, or a variant or fragment thereof.

Preferably, the antibody or antigen-binding fragment thereof comprises a light chain variable (VL) region comprising or consisting of a sequence as substantially set out in SEQ ID No: 188, or a variant or fragment thereof.

Preferably, the antibody or antigen-binding fragment thereof comprises a heavy chain variable region comprising or consisting of SEQ ID No: 175 and a light chain variable region comprising or consisting of SEQ ID No: 188.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in

Preferably, the antibody or antigen-binding fragment thereof comprises a heavy chain constant region comprising or consisting of SEQ ID No: 157 and a light chain constant region comprising or consisting of SEQ ID No: 158.

### 1-D5 H1 L0

Alternatively, in another embodiment, the humanised antibody or antigen-binding fragment thereof is referred to herein as 1-D5_H1_L0.

Preferably, the antibody or antigen-binding fragment thereof comprises a heavy chain variable (VH) region comprising or consisting of a sequence as substantially set out in SEQ ID No: 175, or a variant or fragment thereof.

Preferably, the antibody or antigen-binding fragment thereof comprises a light chain variable (VL) region comprising or consisting of a sequence as substantially set out in SEQ ID No: 187, or a variant or fragment thereof.

Preferably, the antibody or antigen-binding fragment thereof comprises a heavy chain variable region comprising or consisting of SEQ ID No: 175 and a light chain variable region comprising or consisting of SEQ ID No: 187.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-D5 H0 L1

Alternatively, in another embodiment, the humanised antibody or antigen-binding fragment thereof is referred to herein as 1-D5_H0_L1.

Preferably, the antibody or antigen-binding fragment thereof comprises a CDR-H1 domain comprising or consisting of a sequence as substantially set out in SEQ ID No: 173, or a variant or fragment thereof. Preferably, the antibody or antigen-binding fragment thereof comprises a CDR-H2 domain comprising or consisting of a sequence as substantially set out in SEQ ID No: 46, or a variant or fragment thereof. Preferably, the antibody or antigen-binding fragment thereof comprises a CDR-H3 domain comprising or consisting of a sequence as substantially set out in SEQ ID No: 47, or a variant or fragment thereof.

Preferably, the antibody or antigen-binding fragment thereof comprises a CDR-L1 domain comprising or consisting of a sequence as substantially set out in SEQ ID No: 54, or a variant or fragment thereof. Preferably, the antibody or antigen-binding fragment thereof comprises a CDR-L2 domain comprising or consisting of a sequence as substantially set out in SEQ ID No: 55, or a variant or fragment thereof. Preferably, the antibody or antigen-binding fragment thereof comprises a CDR-L3 domain comprising or consisting of a sequence as substantially set out in SEQ ID No: 56, or a variant or fragment thereof.

Preferably, the antibody or antigen-binding fragment thereof comprises a CDR-H1 domain comprising or consisting of SEQ ID No: 173, a CDR-H2 domain comprising or consisting of SEQ ID No: 46; a CDR-H3 domain comprising or consisting of SEQ ID No: 47, a CDR-L1 domain comprising or consisting of SEQ ID No: 54, a CDR-L2 domain comprising or consisting of SEQ ID No: 55, and a CDR-L3 domain comprising or consisting of SEQ ID No: 56.

Preferably, the antibody or antigen-binding fragment thereof comprises a heavy chain variable (VH) region comprising or consisting of a sequence as substantially set out in SEQ ID No: 174, or a variant or fragment thereof.

Preferably, the antibody or antigen-binding fragment thereof comprises a light chain variable (VL) region comprising or consisting of a sequence as substantially set out in SEQ ID No: 188, or a variant or fragment thereof.

Preferably, the antibody or antigen-binding fragment thereof comprises a heavy chain variable region comprising or consisting of SEQ ID No: 174 and a light chain variable region comprising or consisting of SEQ ID No: 188.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-D5 H0 L0

Alternatively, in another embodiment, the humanised antibody or antigen-binding fragment thereof is referred to herein as 1-D5_H0_L0.

Preferably, the antibody or antigen-binding fragment thereof comprises a CDR-H1 domain comprising or consisting of a sequence as substantially set out in SEQ ID No: 173, or a variant or fragment thereof. Preferably, the antibody or antigen-binding fragment thereof comprises a CDR-H2 domain comprising or consisting of a sequence as substantially set out in SEQ ID No: 46, or a variant or fragment thereof. Preferably, the antibody or antigen-binding fragment thereof comprises a CDR-H3 domain comprising or consisting of a sequence as substantially set out in SEQ ID No: 47, or a variant or fragment thereof.

Preferably, the antibody or antigen-binding fragment thereof comprises a CDR-L1 domain comprising or consisting of a sequence as substantially set out in SEQ ID No: 54, or a variant or fragment thereof. Preferably, the antibody or antigen-binding fragment thereof comprises a CDR-L2 domain comprising or consisting of a sequence as substantially set out in SEQ ID No: 55, or a variant or fragment thereof. Preferably, the antibody or antigen-binding fragment thereof comprises a CDR-L3 domain comprising or consisting of a sequence as substantially set out in SEQ ID No: 56, or a variant or fragment thereof.

Preferably, the antibody or antigen-binding fragment thereof comprises a CDR-H1 domain comprising or consisting of SEQ ID No: 173, a CDR-H2 domain comprising or consisting of SEQ ID No: 46; a CDR-H3 domain comprising or consisting of SEQ ID No: 47, a CDR-L1 domain comprising or consisting of SEQ ID No: 54, a CDR-L2 domain comprising or consisting of SEQ ID No: 55, and a CDR-L3 domain comprising or consisting of SEQ ID No: 56.

Preferably, the antibody or antigen-binding fragment thereof comprises a heavy chain variable (VH) region comprising or consisting of a sequence as substantially set out in SEQ ID No: 174, or a variant or fragment thereof.

Preferably, the antibody or antigen-binding fragment thereof comprises a light chain variable (VL) region comprising or consisting of a sequence as substantially set out in SEQ ID No: 187, or a variant or fragment thereof.

Preferably, the antibody or antigen-binding fragment thereof comprises a heavy chain variable region comprising or consisting of SEQ ID No: 174 and a light chain variable region comprising or consisting of SEQ ID No: 187.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 3-H9

In one embodiment, the antibody or antigen-binding fragment thereof is referred to herein as 3-H9. The antibody or antigen-binding fragment thereof may comprise a CDR-H1 domain of SEQ ID No: 63, which is provided herein, as follows:

[SEQ ID No: 63]
GFSLSNYD

Thus, preferably, the antibody or antigen-binding fragment thereof comprises a CDR-H1 domain comprising or consisting of a sequence as substantially set out in SEQ ID No: 63, or a variant or fragment thereof.

The antibody or antigen-binding fragment thereof may comprise a CDR-H2 domain of SEQ ID No: 64, which is provided herein, as follows:

[SEQ ID No: 64]
IHAIGIT

Thus, preferably the antibody or antigen-binding fragment thereof comprises a CDR-H2 domain comprising or consisting of a sequence as substantially set out in SEQ ID No: 64, or a variant or fragment thereof.

The antibody or antigen-binding fragment thereof may comprise a CDR-H3 domain of SEQ ID No: 65, which is provided herein, as follows:

[SEQ ID No: 65]
ARGLVDLNM

Thus, preferably the antibody or antigen-binding fragment thereof comprises a CDR-H3 domain comprising or consisting of a sequence as substantially set out in SEQ ID No: 65, or a variant or fragment thereof.

Preferably, the antibody or antigen-binding fragment thereof comprises a CDR-H1 domain comprising or consisting of SEQ ID No: 63, a CDR-H2 domain comprising or consisting of SEQ ID No: 64 and/or a CDR-H3 domain comprising or consisting of SEQ ID No: 65. Preferably, however, the antibody or antigen-binding fragment thereof comprises a CDR-H1 domain comprising or consisting of SEQ ID No: 63, a CDR-H2 domain comprising or consisting of SEQ ID No: 64 and a CDR-H3 domain comprising or consisting of SEQ ID No: 65.

The antibody or antigen-binding fragment thereof may comprise a FR-H1 domain of SEQ ID No: 66, which is provided herein, as follows:

[SEQ ID No: 66]
SQSLEESGGRLVTPGTPLTLTCSVS

Thus, preferably, the antibody or antigen-binding fragment thereof comprises a FR-H1 domain comprising or consisting of a sequence as substantially set out in SEQ ID No: 66, or a variant or fragment thereof.

The antibody or antigen-binding fragment thereof may comprise a FR-H2 domain of SEQ ID No: 67, which is provided herein, as follows:

[SEQ ID No: 67]
MSWVRQAPGKGLEWIGS

Thus, preferably, the antibody or antigen-binding fragment thereof comprises a FR-H2 domain comprising or consisting of a sequence as substantially set out in SEQ ID No: 67, or a variant or fragment thereof.

The antibody or antigen-binding fragment thereof may comprise a FR-H3 domain of SEQ ID No: 68, which is provided herein, as follows:

[SEQ ID No: 68]
YYANWAEGRFTISKTSTTVDLKMTSLTTEDTATYFC

Thus, preferably, the antibody or antigen-binding fragment thereof comprises a FR-H3 domain comprising or consisting of a sequence as substantially set out in SEQ ID No: 68, or a variant or fragment thereof.

The antibody or antigen-binding fragment thereof may comprise a FR-H4 domain of SEQ ID No: 69, which is provided herein, as follows:

[SEQ ID No: 69]
WGPGTLVTVSS

Thus, preferably, the antibody or antigen-binding fragment thereof comprises a FR-H4 domain comprising or consisting of a sequence as substantially set out in SEQ ID No: 69, or a variant or fragment thereof.

Preferably, the antibody or antigen-binding fragment thereof comprises a FR-H1 domain comprising or consisting of SEQ ID No: 66, a FR-H2 domain comprising or consisting of SEQ ID No: 67, a FR-H3 domain comprising or consisting of SEQ ID No: 68, and/or a FR-H4 domain comprising or consisting of SEQ ID No: 69. Preferably, however, the antibody or antigen-binding fragment thereof comprises a FR-H1 domain comprising or consisting of SEQ ID No: 66, a FR-H2 domain comprising or consisting of SEQ ID No: 67, a FR-H3 domain comprising or consisting of SEQ ID No: 68, and a FR-H4 domain comprising or consisting of SEQ ID No: 69.

The antibody or antigen-binding fragment thereof may comprise a heavy chain variable (VH) sequence as set out in SEQ ID No: 70, which is provided herein, as follows:

[SEQ ID No: 70]
QSLEESGGRLVTPGTPLTLTCSVSGFSLSNYDMSWVRQAPGKGLEWIGS
IHAIGITYYANWAEGRFTISKTSTTVDLKMTSLTTEDTATYFCARGLVD
LNMWGPGTLVTVSS

Preferably, the antibody or antigen-binding fragment thereof comprises a heavy chain variable (VH) region comprising or consisting of a sequence as substantially set out in SEQ ID No: 70, or a variant or fragment thereof.

One embodiment of the nucleotide sequence encoding the heavy chain variable (VH) region is referred to herein as SEQ ID No: 71, as follows:

[SEQ ID No: 71]
CAGTCGCTGGAGGAGTCCGGGGGTCGCCTGGTCACGCCTGGGACACCCT
TGACACTCACCTGTTCAGTCTCTGGATTCTCCCTCAGCAACTACGACAT
GAGCTGGGTCCGCCAGGCTCCAGGGAAGGGACTGGAATGGATCGGGTCC
ATACATGCTATTGGTATCACATACTACGCGAACTGGGCGGAAGGCCGAT
TCACCATCTCCAAAACCTCGACCACGGTGGATCTGAAAATGACCAGTCT
GACAACCGAGGACACGGCCACCTATTTCTGTGCCAGAGGGCTGGTAGAT
TTGAACATGTGGGGCCCGGGCACCCTCGTCACTGTCTCTTCA

Preferably, the antibody or antigen-binding fragment thereof comprises a heavy chain variable (VH) region encoded by a nucleic acid sequence as substantially set out in SEQ ID No: 71, or a variant or fragment thereof.

The antibody or antigen-binding fragment thereof may comprise a CDR-L1 domain of SEQ ID No: 72, which is provided herein, as follows:

[SEQ ID No: 72]
QSVYSNNL

Thus, preferably, the antibody or antigen-binding fragment thereof comprises a CDR-L1 domain comprising or consisting of a sequence as substantially set out in SEQ ID No: 72, or a variant or fragment thereof.

The antibody or antigen-binding fragment thereof may comprise a CDR-L2 domain of SEQ ID No: 73, which is provided herein, as follows:

[SEQ ID No: 73]
DAS

Thus, preferably, the antibody or antigen-binding fragment thereof comprises a CDR-L2 domain comprising or consisting of a sequence as substantially set out in SEQ ID No: 73, or a variant or fragment thereof.

The antibody or antigen-binding fragment thereof may comprise a CDR-L3 domain of SEQ ID No: 74, which is provided herein, as follows:

[SEQ ID No: 74]
QGSYYSSGWYNT

Thus, preferably, the antibody or antigen-binding fragment thereof comprises a CDR-L3 domain comprising or consisting of a sequence as substantially set out in SEQ ID No: 74, or a variant or fragment thereof.

Preferably, the antibody or antigen-binding fragment thereof comprises a CDR-L1 domain comprising or consisting of SEQ ID No: 72, a CDR-L2 domain comprising or consisting of SEQ ID No: 73, and/or a CDR-L3 domain comprising or consisting of SEQ ID No: 74. However, preferably the antibody or antigen-binding fragment thereof comprises a CDR-L1 domain comprising or consisting of SEQ ID No: 72, a CDR-L2 domain comprising or consisting of SEQ ID No: 73, and a CDR-L3 domain comprising or consisting of SEQ ID No: 74.

The antibody or antigen-binding fragment thereof may comprise a FR-L1 domain of SEQ ID No: 75, which is provided herein, as follows:

[SEQ ID No: 75]
AIKMTQTPSSVSVAVGGTVTINCQSS

Thus, preferably, the antibody or antigen-binding fragment thereof comprises a FR-L1 domain comprising or consisting of a sequence as substantially set out in SEQ ID No: 75, or a variant or fragment thereof.

The antibody or antigen-binding fragment thereof may comprise a FR-L2 domain of SEQ ID No: 76, which is provided herein, as follows:

[SEQ ID No: 76]
LSWYQQKPGQPPKLLIY

Thus, preferably, the antibody or antigen-binding fragment thereof comprises a FR-L2 domain comprising or consisting of a sequence as substantially set out in SEQ ID No: 76, or a variant or fragment thereof.

The antibody or antigen-binding fragment thereof may comprise a FR-L3 domain of SEQ ID No: 77, which is provided herein, as follows:

[SEQ ID No: 77]
TLESGVPSRFKGSGSGTQFTLTISGVQCEDAATYYC

Thus, preferably, the antibody or antigen-binding fragment thereof comprises a FR-L3 domain comprising or consisting of a sequence as substantially set out in SEQ ID No: 77, or a variant or fragment thereof.

The antibody or antigen-binding fragment thereof may comprise a FR-L4 domain of SEQ ID No: 78, which is provided herein, as follows:

[SEQ ID No: 78]
FGGGTEVVVE

Thus, preferably, the antibody or antigen-binding fragment thereof comprises a FR-L4 domain comprising or consisting of a sequence as substantially set out in SEQ ID No: 78, or a variant or fragment thereof.

Preferably, the antibody or antigen-binding fragment thereof comprises a FR-L1 domain comprising or consisting of SEQ ID No: 75, a FR-L2 domain comprising or consisting of SEQ ID No: 76, a FR-L3 domain comprising or consisting of SEQ ID No: 77, and/or a FR-L4 domain comprising or consisting of SEQ ID No: 78. Preferably, however, the antibody or antigen-binding fragment thereof comprises a FR-L1 domain comprising or consisting of SEQ ID No: 75, a FR-L2 domain comprising or consisting of SEQ ID No: 76, a FR-L3 domain comprising or consisting of SEQ ID No: 77, and a FR-L4 domain comprising or consisting of SEQ ID No: 78.

The antibody or antigen-binding fragment thereof may comprise a light chain variable (VL) sequence as set out in SEQ ID No: 79, which is provided herein, as follows:

[SEQ ID No: 79]
AIKMTQTPSSVSVAVGGTVTINCQSSQSVYSNNLLSWYQQKPGQPPKLL
IYDASTLESGVPSRFKGSGSGTQFTLTISGVQCEDAATYYCQGSYYSSG
WYNTFGGGTEVVVE

Preferably, the antibody or antigen-binding fragment thereof comprises a light chain variable region comprising or consisting of a sequence as substantially set out in SEQ ID No: 79, or a variant or fragment thereof.

One embodiment of the nucleotide sequence encoding the light chain variable (VL) region is referred to herein as SEQ ID No: 80, as follows:

[SEQ ID No: 80]
GCTATTAAAATGACCCAGACTCCATCGTCCGTGTCTGTAGCTGTGGGAG
GCACAGTCACCATCAATTGCCAGTCCAGTCAGAGTGTTTATAGTAACAA
CCTCTTATCTTGGTACCAGCAGAAACCAGGGCAGCCTCCCAAGCTCTTG
ATCTACGATGCATCCACTCTGGAATCTGGGGTCCCATCGCGGTTCAAAG
GCAGTGGATCTGGGACACAGTTCACTCTCACCATCAGCGGCGTGCAGTG
TGAGGATGCTGCCACTTACTACTGTCAAGGCAGTTATTATAGTAGTGGT
TGGTACAATACTTTCGGCGGAGGGACCGAGGTGGTCGTCGAA

Preferably, the antibody or antigen-binding fragment thereof comprises a light chain variable (VL) region encoded by a nucleic acid sequence as substantially set out in SEQ ID No: 80, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 63, a CDR-H2 domain comprising or consisting of SEQ ID No: 64; a CDR-H3 domain comprising or consisting of SEQ ID No: 65, a CDR-L1 domain comprising or consisting of SEQ ID No: 72, a CDR-L2 domain comprising or consisting of SEQ ID No: 73, and a CDR-L3 domain comprising or consisting of SEQ ID No: 74.

Preferably, the antibody or antigen-binding fragment thereof comprises a heavy chain variable region comprising or consisting of SEQ ID No: 70, and a light chain variable region comprising or consisting of SEQ ID No: 79.

Preferably, the antibody or antigen-binding fragment thereof comprises a heavy chain variable region encoded by a nucleic acid sequence comprising or consisting of SEQ ID No: 71, and a light chain variable region encoded by a nucleic acid sequence comprising or consisting of SEQ ID No: 80.

The inventors then set out to generate humanised antibodies of 3-H9, and the sequences of the humanised antibodies are illustrated in FIG. 10. Unless stated otherwise, the six CDR sequences of the humanised antibodies are identical to the six CDR sequences of the parental antibody 3-H9.

3-H9 Parental (hIgG1) In one embodiment, the humanised antibody or antigen-binding fragment thereof is referred to herein as 3-H9_parental (hIgG1).

Preferably, the antibody or antigen-binding fragment thereof comprises a heavy chain variable (VH) region comprising or consisting of a sequence as substantially set out in SEQ ID No: 160, or a variant or fragment thereof.

Preferably, the antibody or antigen-binding fragment thereof comprises a light chain variable (VL) region comprising or consisting of a sequence as substantially set out in SEQ ID No: 79, or a variant or fragment thereof.

Preferably, the antibody or antigen-binding fragment thereof comprises a heavy chain variable region comprising or consisting of SEQ ID No: 160 and a light chain variable region comprising or consisting of SEQ ID No: 79.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

3-H9 Parental (hIgG1-L234A-L235A-P329G)

Alternatively, in another embodiment, the humanised antibody or antigen-binding fragment thereof is referred to herein as 3-H9_parental (hIgG1-L234A-L235A-P329G).

Preferably, the antibody or antigen-binding fragment thereof comprises a heavy chain variable (VH) region comprising or consisting of a sequence as substantially set out in SEQ ID No: 160, or a variant or fragment thereof.

Preferably, the antibody or antigen-binding fragment thereof comprises a light chain variable (VL) region comprising or consisting of a sequence as substantially set out in SEQ ID No: 79, or a variant or fragment thereof.

Preferably, the antibody or antigen-binding fragment thereof comprises a heavy chain variable region comprising or consisting of SEQ ID No: 160 and a light chain variable region comprising or consisting of SEQ ID No: 79.

Preferably, the antibody or antigen-binding fragment thereof comprises a heavy chain constant (HC) region comprising or consisting of a sequence as substantially set out in SEQ ID No: 162, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 162 and a light chain constant region comprising or consisting of SEQ ID No: 158.

3-H9 Parental (hIgG1-Fab)

Alternatively, in another embodiment, the humanised antibody or antigen-binding fragment thereof is referred to herein as 3-H9_parental (hIgG1-Fab).

Preferably, the antibody or antigen-binding fragment thereof comprises a heavy chain variable (VH) region comprising or consisting of a sequence as substantially set out in SEQ ID No: 160, or a variant or fragment thereof.

Preferably, the antibody or antigen-binding fragment thereof comprises a light chain variable (VL) region comprising or consisting of a sequence as substantially set out in SEQ ID No: 79, or a variant or fragment thereof.

Preferably, the antibody or antigen-binding fragment thereof comprises a heavy chain variable region comprising or consisting of SEQ ID No: 160 and a light chain variable region comprising or consisting of SEQ ID No: 79.

Preferably, the antibody or antigen-binding fragment thereof comprises a heavy chain constant (HC) region comprising or consisting of a sequence as substantially set out in SEQ ID No: 163, or a variant or fragment thereof.

Preferably, the antibody or antigen-binding fragment thereof comprises a light chain constant (LC) region comprising or consisting of a sequence as substantially set out in

Preferably, the antibody or antigen-binding fragment thereof comprises a heavy chain constant region comprising or consisting of SEQ ID No: 163 and a light chain constant region comprising or consisting of SEQ ID No: 158.

### 3-H9 H0 (Higg1)

Alternatively, in another embodiment, the humanised antibody or antigen-binding fragment thereof is referred to herein as 3-H9_H0 (hIgG1).

Preferably, the antibody or antigen-binding fragment thereof comprises a CDR-H1 domain comprising or consisting of a sequence as substantially set out in SEQ ID No: 177, or a variant or fragment thereof. Preferably, the antibody or antigen-binding fragment thereof comprises a CDR-H2 domain comprising or consisting of a sequence as substantially set out in SEQ ID No: 64, or a variant or fragment thereof. Preferably, the antibody or antigen-binding fragment thereof comprises a CDR-H3 domain comprising or consisting of a sequence as substantially set out in SEQ ID No: 65, or a variant or fragment thereof.

Preferably, the antibody or antigen-binding fragment thereof comprises a CDR-L1 domain comprising or consisting of a sequence as substantially set out in SEQ ID No: 72, or a variant or fragment thereof. Preferably, the antibody or antigen-binding fragment thereof comprises a CDR-L2 domain comprising or consisting of a sequence as substantially set out in SEQ ID No: 73, or a variant or fragment thereof. Preferably, the antibody or antigen-binding fragment thereof comprises a CDR-L3 domain comprising or consisting of a sequence as substantially set out in SEQ ID No: 74, or a variant or fragment thereof.

Preferably, the antibody or antigen-binding fragment thereof comprises a CDR-H1 domain comprising or consisting of SEQ ID No: 177, a CDR-H2 domain comprising or consisting of SEQ ID No: 64; a CDR-H3 domain comprising or consisting of SEQ ID No: 65, a CDR-L1 domain comprising or consisting of SEQ ID No: 72, a CDR-L2 domain comprising or consisting of SEQ ID No: 73, and a CDR-L3 domain comprising or consisting of SEQ ID No: 74.

Preferably, the antibody or antigen-binding fragment thereof comprises a heavy chain variable (VH) region comprising or consisting of a sequence as substantially set out in SEQ ID No: 178, or a variant or fragment thereof.

Preferably, the antibody or antigen-binding fragment thereof comprises a light chain variable (VL) region comprising or consisting of a sequence as substantially set out in SEQ ID No: 79, or a variant or fragment thereof.

Preferably, the antibody or antigen-binding fragment thereof comprises a heavy chain variable region comprising or consisting of SEQ ID No: 178 and a light chain variable region comprising or consisting of SEQ ID No: 79.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

3-H9 H1 (hIgG1)

Alternatively, in another embodiment, the humanised antibody or antigen-binding fragment thereof is referred to herein as 3-H9_H1 (hIgG1).

Preferably, the antibody or antigen-binding fragment thereof comprises a heavy chain variable (VH) region comprising or consisting of a sequence as substantially set out in SEQ ID No: 179, or a variant or fragment thereof.

Preferably, the antibody or antigen-binding fragment thereof comprises a light chain variable (VL) region comprising or consisting of a sequence as substantially set out in SEQ ID No: 79, or a variant or fragment thereof.

Preferably, the antibody or antigen-binding fragment thereof comprises a heavy chain variable region comprising or consisting of SEQ ID No: 179 and a light chain variable region comprising or consisting of SEQ ID No: 79.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

3-H9 H2 (hIgG1)

Alternatively, in another embodiment, the humanised antibody or antigen-binding fragment thereof is referred to herein as 3-H9_H2 (hIgG1).

Preferably, the antibody or antigen-binding fragment thereof comprises a heavy chain variable (VH) region comprising or consisting of a sequence as substantially set out in SEQ ID No: 180, or a variant or fragment thereof.

Preferably, the antibody or antigen-binding fragment thereof comprises a light chain variable (VL) region comprising or consisting of a sequence as substantially set out in SEQ ID No: 79, or a variant or fragment thereof.

Preferably, the antibody or antigen-binding fragment thereof comprises a heavy chain variable region comprising or consisting of SEQ ID No: 180 and a light chain variable region comprising or consisting of SEQ ID No: 79.

Preferably, the antibody or antigen-binding fragment thereof comprises a heavy chain constant (HC) region comprising or consisting of a sequence as substantially set out in

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

3-H9 L0 (hIgG1K)

Alternatively, in another embodiment, the humanised antibody or antigen-binding fragment thereof is referred to herein as 3-H9_L0 (hIgG1K).

Preferably, the antibody or antigen-binding fragment thereof comprises a heavy chain variable (VH) region comprising or consisting of a sequence as substantially set out in SEQ ID No: 160, or a variant or fragment thereof.

Preferably, the antibody or antigen-binding fragment thereof comprises a light chain variable (VL) region comprising or consisting of a sequence as substantially set out in SEQ ID No: 189, or a variant or fragment thereof.

Preferably, the antibody or antigen-binding fragment thereof comprises a heavy chain variable region comprising or consisting of SEQ ID No: 160 and a light chain variable region comprising or consisting of SEQ ID No: 189.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain 3-H9 H2 L0

Alternatively, in another embodiment, the humanised antibody or antigen-binding fragment thereof is referred to herein as 3-H9_H2_L0.

Preferably, the antibody or antigen-binding fragment thereof comprises a heavy chain variable (VH) region comprising or consisting of a sequence as substantially set out in SEQ ID No: 180, or a variant or fragment thereof.

Preferably, the antibody or antigen-binding fragment thereof comprises a light chain variable (VL) region comprising or consisting of a sequence as substantially set out in SEQ ID No: 189, or a variant or fragment thereof.

Preferably, the antibody or antigen-binding fragment thereof comprises a heavy chain variable region comprising or consisting of SEQ ID No: 180 and a light chain variable region comprising or consisting of SEQ ID No: 189.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 3-H9 H1 L0

Alternatively, in another embodiment, the humanised antibody or antigen-binding fragment thereof is referred to herein as 3-H9_H1_L0.

Preferably, the antibody or antigen-binding fragment thereof comprises a heavy chain variable (VH) region comprising or consisting of a sequence as substantially set out in SEQ ID No: 179, or a variant or fragment thereof.

Preferably, the antibody or antigen-binding fragment thereof comprises a light chain variable (VL) region comprising or consisting of a sequence as substantially set out in SEQ ID No: 189, or a variant or fragment thereof.

Preferably, the antibody or antigen-binding fragment thereof comprises a heavy chain variable region comprising or consisting of SEQ ID No: 179 and a light chain variable region comprising or consisting of SEQ ID No: 189.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 3-H9 H0 L0

Alternatively, in another embodiment, the humanised antibody or antigen-binding fragment thereof is referred to herein as 3-H9_H0_L0.

Preferably, the antibody or antigen-binding fragment thereof comprises a CDR-H1 domain comprising or consisting of a sequence as substantially set out in SEQ ID No: 177, or a variant or fragment thereof. Preferably, the antibody or antigen-binding fragment thereof comprises a CDR-H2 domain comprising or consisting of a sequence as substantially set out in SEQ ID No: 64, or a variant or fragment thereof. Preferably, the antibody or antigen-binding fragment thereof comprises a CDR-H3 domain comprising or consisting of a sequence as substantially set out in SEQ ID No: 65, or a variant or fragment thereof.

Preferably, the antibody or antigen-binding fragment thereof comprises a CDR-L1 domain comprising or consisting of a sequence as substantially set out in SEQ ID No: 72, or a variant or fragment thereof. Preferably, the antibody or antigen-binding fragment thereof comprises a CDR-L2 domain comprising or consisting of a sequence as substantially set out in SEQ ID No: 73, or a variant or fragment thereof. Preferably, the antibody or antigen-binding fragment thereof comprises a CDR-L3 domain comprising or consisting of a sequence as substantially set out in SEQ ID No: 74, or a variant or fragment thereof.

Preferably, the antibody or antigen-binding fragment thereof comprises a CDR-H1 domain comprising or consisting of SEQ ID No: 177, a CDR-H2 domain comprising or consisting of SEQ ID No: 64; a CDR-H3 domain comprising or consisting of SEQ ID

No: 65, a CDR-L1 domain comprising or consisting of SEQ ID No: 72, a CDR-L2 domain comprising or consisting of SEQ ID No: 73, and a CDR-L3 domain comprising or consisting of SEQ ID No: 74.

Preferably, the antibody or antigen-binding fragment thereof comprises a heavy chain variable (VH) region comprising or consisting of a sequence as substantially set out in SEQ ID No: 178, or a variant or fragment thereof.

Preferably, the antibody or antigen-binding fragment thereof comprises a light chain variable (VL) region comprising or consisting of a sequence as substantially set out in SEQ ID No: 189, or a variant or fragment thereof.

Preferably, the antibody or antigen-binding fragment thereof comprises a heavy chain variable region comprising or consisting of SEQ ID No: 178 and a light chain variable region comprising or consisting of SEQ ID No: 189.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain

### 1-G5

In one embodiment, the antibody or antigen-binding fragment thereof is referred to herein as 1-G5. The antibody or antigen-binding fragment thereof may comprise a CDR-H1 domain of SEQ ID No: 81, which is provided herein, as follows:

[SEQ ID No: 81]
GFSLSSYD

Thus, preferably, the antibody or antigen-binding fragment thereof comprises a CDR-H1 domain comprising or consisting of a sequence as substantially set out in SEQ ID No: 81, or a variant or fragment thereof.

The antibody or antigen-binding fragment thereof may comprise a CDR-H2 domain of SEQ ID No: 82, which is provided herein, as follows:

[SEQ ID No: 82]
IHATGIT

Thus, preferably the antibody or antigen-binding fragment thereof comprises a CDR-H2 domain comprising or consisting of a sequence as substantially set out in SEQ ID No: 82, or a variant or fragment thereof.

The antibody or antigen-binding fragment thereof may comprise a CDR-H3 domain of SEQ ID No: 83, which is provided herein, as follows:

[SEQ ID No: 83]
ARGLVDLNM

Thus, preferably the antibody or antigen-binding fragment thereof comprises a CDR-H3 domain comprising or consisting of a sequence as substantially set out in SEQ ID No: 83, or a variant or fragment thereof.

Preferably, the antibody or antigen-binding fragment thereof comprises a CDR-H1 domain comprising or consisting of SEQ ID No: 81, a CDR-H2 domain comprising or consisting of SEQ ID No: 82 and/or a CDR-H3 domain comprising or consisting of SEQ ID No: 83. Preferably, however, the antibody or antigen-binding fragment thereof comprises a CDR-H1 domain comprising or consisting of SEQ ID No: 81, a CDR-H2 domain comprising or consisting of SEQ ID No: 82 and a CDR-H3 domain comprising or consisting of SEQ ID No: 83.

The antibody or antigen-binding fragment thereof may comprise a FR-H1 domain of SEQ ID No: 84, which is provided herein, as follows:

[SEQ ID No: 84]
SQSLEESGGRLVTPGTPLTLTCSVS

Thus, preferably, the antibody or antigen-binding fragment thereof comprises a FR-H1 domain comprising or consisting of a sequence as substantially set out in SEQ ID No: 84, or a variant or fragment thereof.

The antibody or antigen-binding fragment thereof may comprise a FR-H2 domain of SEQ ID No: 85, which is provided herein, as follows:

[SEQ ID No: 85]
MTWVRQAPGKGLEWIGS

Thus, preferably, the antibody or antigen-binding fragment thereof comprises a FR-H2 domain comprising or consisting of a sequence as substantially set out in SEQ ID No: 85, or a variant or fragment thereof.

The antibody or antigen-binding fragment thereof may comprise a FR-H3 domain of SEQ ID No: 86, which is provided herein, as follows:

[SEQ ID No: 86]
FYANWAKGRFTTSKTSTTVDLKMTSLTTEDTATYFC

Thus, preferably, the antibody or antigen-binding fragment thereof comprises a FR-H3 domain comprising or consisting of a sequence as substantially set out in SEQ ID No: 86, or a variant or fragment thereof.

The antibody or antigen-binding fragment thereof may comprise a FR-H4 domain of SEQ ID No: 87, which is provided herein, as follows:

[SEQ ID No: 87]
WGPGTLVTVSS

Thus, preferably, the antibody or antigen-binding fragment thereof comprises a FR-H4 domain comprising or consisting of a sequence as substantially set out in SEQ ID No: 87, or a variant or fragment thereof.

Preferably, the antibody or antigen-binding fragment thereof comprises a FR-H1 domain comprising or consisting of SEQ ID No: 84, a FR-H2 domain comprising or consisting of SEQ ID No: 85, a FR-H3 domain comprising or consisting of SEQ ID No: 86, and/or a FR-H4 domain comprising or consisting of SEQ ID No: 87. Preferably, however, the antibody or antigen-binding fragment thereof comprises a FR-H1 domain comprising or consisting of SEQ ID No: 84, a FR-H2 domain comprising or consisting of SEQ ID No: 85, a FR-H3 domain comprising or consisting of SEQ ID No: 86, and a FR-H4 domain comprising or consisting of SEQ ID No: 87.

The antibody or antigen-binding fragment thereof may comprise a heavy chain variable (VH) sequence as set out in SEQ ID No: 88, which is provided herein, as follows:

[SEQ ID No: 88]
QSLEESGGRLVTPGTPLTLTCSVSGFSLSSYDMTWVRQAPGKGLEWIGS
IHATGITFYANWAKGRFTTSKTSTTVDLKMTSLTTEDTATYFCARGLVD
LNMWGPGTLVTVSS

Preferably, the antibody or antigen-binding fragment thereof comprises a heavy chain variable (VH) region comprising or consisting of a sequence as substantially set out in SEQ ID No: 88, or a variant or fragment thereof.

One embodiment of the nucleotide sequence encoding the heavy chain variable (VH) region is referred to herein as SEQ ID No: 89, as follows:

[SEQ ID No: 89]
CAGTCGTTGGAGGAGTCCGGGGGTCGCCTGGTCACGCCTGGGACACCCT
TGACACTCACCTGTTCAGTCTCTGGATTCTCCCTCAGCAGCTACGACAT
GACCTGGGTCCGCCAGGCTCCAGGGAAGGGGCTGGAATGGATCGGGTCC
ATACATGCTACTGGTATCACATTCTACGCGAACTGGGCGAAAGGCCGAT
TCACCACCTCCAAAACCTCGACCACGGTGGATCTGAAAATGACCAGTCT
GACAACCGAGGACACGGCCACCTATTTCTGTGCCAGAGGGCTGGTAGAT
TTGAACATGTGGGGCCCGGGCACCCTCGTCACCGTCTCTTCA

Preferably, the antibody or antigen-binding fragment thereof comprises a heavy chain variable (VH) region encoded by a nucleic acid sequence as substantially set out in SEQ ID No: 89, or a variant or fragment thereof.

The antibody or antigen-binding fragment thereof may comprise a CDR-L1 domain of SEQ ID No: 90, which is provided herein, as follows:

[SEQ ID No: 90]
QSVYNNNY

Thus, preferably, the antibody or antigen-binding fragment thereof comprises a CDR-L1 domain comprising or consisting of a sequence as substantially set out in SEQ ID No: 90, or a variant or fragment thereof.

The antibody or antigen-binding fragment thereof may comprise a CDR-L2 domain of SEQ ID No: 91, which is provided herein, as follows:

[SEQ ID No: 91]
DAS

Thus, preferably, the antibody or antigen-binding fragment thereof comprises a CDR-L2 domain comprising or consisting of a sequence as substantially set out in SEQ ID No: 91, or a variant or fragment thereof.

The antibody or antigen-binding fragment thereof may comprise a CDR-L3 domain of SEQ ID No: 92, which is provided herein, as follows:

[SEQ ID No: 92]
QGSYYSGGWDTA

Thus, preferably, the antibody or antigen-binding fragment thereof comprises a CDR-L3 domain comprising or consisting of a sequence as substantially set out in SEQ ID No: 92, or a variant or fragment thereof.

Preferably, the antibody or antigen-binding fragment thereof comprises a CDR-L1 domain comprising or consisting of SEQ ID No: 90, a CDR-L2 domain comprising or consisting of SEQ ID No: 91, and/or a CDR-L3 domain comprising or consisting of SEQ ID No: 92. However, preferably the antibody or antigen-binding fragment thereof comprises a CDR-L1 domain comprising or consisting of SEQ ID No: 90, a CDR-L2 domain comprising or consisting of SEQ ID No: 91, and a CDR-L3 domain comprising or consisting of SEQ ID No: 92.

The antibody or antigen-binding fragment thereof may comprise a FR-L1 domain of SEQ ID No: 93, which is provided herein, as follows:

[SEQ ID No: 93]
DPVMTQTASSVSAAVGGTVTINCQAS

Thus, preferably, the antibody or antigen-binding fragment thereof comprises a FR-L1 domain comprising or consisting of a sequence as substantially set out in SEQ ID No: 93, or a variant or fragment thereof.

The antibody or antigen-binding fragment thereof may comprise a FR-L2 domain of SEQ ID No: 94, which is provided herein, as follows:

[SEQ ID No: 94]
LSWYQQKPGQPPKLLIY

Thus, preferably, the antibody or antigen-binding fragment thereof comprises a FR-L2 domain comprising or consisting of a sequence as substantially set out in SEQ ID No: 94, or a variant or fragment thereof.

The antibody or antigen-binding fragment thereof may comprise a FR-L3 domain of SEQ ID No: 95, which is provided herein, as follows:

[SEQ ID No: 95]
TLASGVPSRFSGNGSGTQFTLTISGVQCDDAATYYC

Thus, preferably, the antibody or antigen-binding fragment thereof comprises a FR-L3 domain comprising or consisting of a sequence as substantially set out in SEQ ID No: 95, or a variant or fragment thereof.

The antibody or antigen-binding fragment thereof may comprise a FR-L4 domain of SEQ ID No: 96, which is provided herein, as follows:

[SEQ ID No: 96]
FGGGTKVVVK

Thus, preferably, the antibody or antigen-binding fragment thereof comprises a FR-L4 domain comprising or consisting of a sequence as substantially set out in SEQ ID No: 96, or a variant or fragment thereof.

Preferably, the antibody or antigen-binding fragment thereof comprises a FR-L1 domain comprising or consisting of SEQ ID No: 93, a FR-L2 domain comprising or consisting of SEQ ID No: 94, a FR-L3 domain comprising or consisting of SEQ ID No: 95, and/or a FR-L4 domain comprising or consisting of SEQ ID No: 96. Preferably, however, the antibody or antigen-binding fragment thereof comprises a FR-L1 domain comprising or consisting of SEQ ID No: 93, a FR-L2 domain comprising or consisting of SEQ ID No: 94, a FR-L3 domain comprising or consisting of SEQ ID No: 95, and a FR-L4 domain comprising or consisting of SEQ ID No: 96.

The antibody or antigen-binding fragment thereof may comprise a light chain variable (VL) sequence as set out in SEQ ID No: 97, which is provided herein, as follows:

[SEQ ID No: 97]
DPVMTQTASSVSAAVGGTVTINCQASQSVYNNNYLSWYQQKPGQPPKLL
IYDASTLASGVPSRFSGNGSGTQFTLTISGVQCDDAATYYCQGSYYSGG
WDTAFGGGTKVVVK

Preferably, the antibody or antigen-binding fragment thereof comprises a light chain variable region comprising or consisting of a sequence as substantially set out in SEQ ID No: 97, or a variant or fragment thereof.

One embodiment of the nucleotide sequence encoding the light chain variable (VL) region is referred to herein as SEQ ID No: 98, as follows:

[SEQ ID No: 98]
GATCCCGTGATGACCCAGACTGCGTCCTCCGTGTCTGCAGCTGTGGGAG
GCACAGTCACCATCAATTGCCAGGCCAGTCAGAGTGTTTATAATAACAA
CTACTTATCCTGGTATCAGCAGAAACCAGGGCAGCCTCCCAAGCTCTTG
ATCTACGATGCATCCACTCTGGCATCTGGGGTCCCATCCCGGTTCAGCG
GCAATGGATCTGGGACACAGTTCACTCTCACCATCAGCGGCGTACAGTG
TGACGATGCTGCCACTTACTACTGTCAAGGCAGTTATTATAGTGGTGGT
TGGGACACTGCTTTCGGCGGAGGGACCAAGGTGGTCGTCAAA

Preferably, the antibody or antigen-binding fragment thereof comprises a light chain variable (VL) region encoded by a nucleic acid sequence as substantially set out in SEQ ID No: 98, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 81, a CDR-H2 domain comprising or consisting of SEQ ID No: 82; a CDR-H3 domain comprising or consisting of SEQ ID

No: 83, a CDR-L1 domain comprising or consisting of SEQ ID No: 90, a CDR-L2 domain comprising or consisting of SEQ ID No: 91, and a CDR-L3 domain comprising or consisting of SEQ ID No: 92.

Preferably, the antibody or antigen-binding fragment thereof comprises a heavy chain variable region comprising or consisting of SEQ ID No: 88, and a light chain variable region comprising or consisting of SEQ ID No: 97.

Preferably, the antibody or antigen-binding fragment thereof comprises a heavy chain variable region encoded by a nucleic acid sequence comprising or consisting of SEQ ID

No: 89, and a light chain variable region encoded by a nucleic acid sequence comprising or consisting of SEQ ID No: 98.

The inventors then set out to generate humanised antibodies of 1-G5, and the sequences of the humanised antibodies are illustrated in FIG. 10. Unless stated otherwise, the six CDR sequences of the humanised antibodies are identical to the six CDR sequences of the parental antibody 1-G5.

1-G5 Parental (hIgG1)

In one embodiment, the humanised antibody or antigen-binding fragment thereof is referred to herein as 1-G5_parental (hIgG1).

Preferably, the antibody or antigen-binding fragment thereof comprises a heavy chain variable (VH) region comprising or consisting of a sequence as substantially set out in SEQ ID No: 161, or a variant or fragment thereof.

Preferably, the antibody or antigen-binding fragment thereof comprises a light chain variable (VL) region comprising or consisting of a sequence as substantially set out in SEQ ID No: 97, or a variant or fragment thereof.

Preferably, the antibody or antigen-binding fragment thereof comprises a heavy chain variable region comprising or consisting of SEQ ID No: 161 and a light chain variable region comprising or consisting of SEQ ID No: 97.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

1-G5 Parental (hIgG1-L234A-L235A-P329G)

Alternatively, in another embodiment, the humanised antibody or antigen-binding fragment thereof is referred to herein as 1-G5_parental (hIgG1-L234A-L235A-P329G).

Preferably, the antibody or antigen-binding fragment thereof comprises a heavy chain variable (VH) region comprising or consisting of a sequence as substantially set out in SEQ ID No: 161, or a variant or fragment thereof.

Preferably, the antibody or antigen-binding fragment thereof comprises a light chain variable (VL) region comprising or consisting of a sequence as substantially set out in SEQ ID No: 97, or a variant or fragment thereof.

Preferably, the antibody or antigen-binding fragment thereof comprises a heavy chain variable region comprising or consisting of SEQ ID No: 161 and a light chain variable region comprising or consisting of SEQ ID No: 97.

Preferably, the antibody or antigen-binding fragment thereof comprises a heavy chain constant (HC) region comprising or consisting of a sequence as substantially set out in SEQ ID No: 162, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 162 and a light chain constant region comprising or consisting of SEQ ID No: 158.

1-G5 Parental (hIgG1-Fab)

Alternatively, in another embodiment, the humanised antibody or antigen-binding fragment thereof is referred to herein as 1-G5_parental (hIgG1-Fab).

Preferably, the antibody or antigen-binding fragment thereof comprises a heavy chain variable (VH) region comprising or consisting of a sequence as substantially set out in SEQ ID No: 161, or a variant or fragment thereof.

Preferably, the antibody or antigen-binding fragment thereof comprises a light chain variable (VL) region comprising or consisting of a sequence as substantially set out in SEQ ID No: 97, or a variant or fragment thereof.

Preferably, the antibody or antigen-binding fragment thereof comprises a heavy chain variable region comprising or consisting of SEQ ID No: 161 and a light chain variable region comprising or consisting of SEQ ID No: 97.

Preferably, the antibody or antigen-binding fragment thereof comprises a heavy chain constant (HC) region comprising or consisting of a sequence as substantially set out in SEQ ID No: 163, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 163 and a light chain constant region comprising or consisting of SEQ ID No: 158.

1-G5 H0 (hIgG1)

Alternatively, in another embodiment, the humanised antibody or antigen-binding fragment thereof is referred to herein as 1-G5_H0 (hIgG1).

Preferably, the antibody or antigen-binding fragment thereof comprises a CDR-H1 domain comprising or consisting of a sequence as substantially set out in SEQ ID No: 181, or a variant or fragment thereof. Preferably, the antibody or antigen-binding fragment thereof comprises a CDR-H2 domain comprising or consisting of a sequence as substantially set out in SEQ ID No: 82, or a variant or fragment thereof. Preferably, the antibody or antigen-binding fragment thereof comprises a CDR-H3 domain comprising or consisting of a sequence as substantially set out in SEQ ID No: 83, or a variant or fragment thereof.

Preferably, the antibody or antigen-binding fragment thereof comprises a CDR-L1 domain comprising or consisting of a sequence as substantially set out in SEQ ID No: 90, or a variant or fragment thereof. Preferably, the antibody or antigen-binding fragment thereof comprises a CDR-L2 domain comprising or consisting of a sequence as substantially set out in SEQ ID No: 91, or a variant or fragment thereof. Preferably, the antibody or antigen-binding fragment thereof comprises a CDR-L3 domain comprising or consisting of a sequence as substantially set out in SEQ ID No: 92, or a variant or fragment thereof.

Preferably, the antibody or antigen-binding fragment thereof comprises a CDR-H1 domain comprising or consisting of SEQ ID No: 181, a CDR-H2 domain comprising or consisting of SEQ ID No: 82; a CDR-H3 domain comprising or consisting of SEQ ID No: 83, a CDR-L1 domain comprising or consisting of SEQ ID No: 90, a CDR-L2 domain comprising or consisting of SEQ ID No: 91, and a CDR-L3 domain comprising or consisting of SEQ ID No: 92.

Preferably, the antibody or antigen-binding fragment thereof comprises a heavy chain variable (VH) region comprising or consisting of a sequence as substantially set out in SEQ ID No: 182, or a variant or fragment thereof.

Preferably, the antibody or antigen-binding fragment thereof comprises a light chain variable (VL) region comprising or consisting of a sequence as substantially set out in SEQ ID No: 97, or a variant or fragment thereof.

Preferably, the antibody or antigen-binding fragment thereof comprises a heavy chain variable region comprising or consisting of SEQ ID No: 182 and a light chain variable region comprising or consisting of SEQ ID No: 97.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

1-G5 H1 (hIgG1)

Alternatively, in another embodiment, the humanised antibody or antigen-binding fragment thereof is referred to herein as 1-G5_H1 (hIgG1).

Preferably, the antibody or antigen-binding fragment thereof comprises a heavy chain variable (VH) region comprising or consisting of a sequence as substantially set out in SEQ ID No: 183, or a variant or fragment thereof.

Preferably, the antibody or antigen-binding fragment thereof comprises a light chain variable (VL) region comprising or consisting of a sequence as substantially set out in SEQ ID No: 97, or a variant or fragment thereof.

Preferably, the antibody or antigen-binding fragment thereof comprises a heavy chain variable region comprising or consisting of SEQ ID No: 183 and a light chain variable region comprising or consisting of SEQ ID No: 97.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

1-G5 H2 (hIgG1)

Alternatively, in another embodiment, the humanised antibody or antigen-binding fragment thereof is referred to herein as 1-G5_H2 (hIgG1).

Preferably, the antibody or antigen-binding fragment thereof comprises a heavy chain variable (VH) region comprising or consisting of a sequence as substantially set out in SEQ ID No: 184, or a variant or fragment thereof.

Preferably, the antibody or antigen-binding fragment thereof comprises a light chain variable (VL) region comprising or consisting of a sequence as substantially set out in SEQ ID No: 97, or a variant or fragment thereof.

Preferably, the antibody or antigen-binding fragment thereof comprises a heavy chain variable region comprising or consisting of SEQ ID No: 184 and a light chain variable region comprising or consisting of SEQ ID No: 97.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

1-G5 L0 (hIgG1K)

Alternatively, in another embodiment, the humanised antibody or antigen-binding fragment thereof is referred to herein as 1-G5_L0 (hIgG1K).

Preferably, the antibody or antigen-binding fragment thereof comprises a heavy chain variable (VH) region comprising or consisting of a sequence as substantially set out in SEQ ID No: 161, or a variant or fragment thereof.

Preferably, the antibody or antigen-binding fragment thereof comprises a light chain variable (VL) region comprising or consisting of a sequence as substantially set out in SEQ ID No: 192, or a variant or fragment thereof.

Preferably, the antibody or antigen-binding fragment thereof comprises a heavy chain variable region comprising or consisting of SEQ ID No: 161 and a light chain variable region comprising or consisting of SEQ ID No: 192.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in

Preferably, the antibody or antigen-binding fragment thereof comprises a heavy chain constant region comprising or consisting of SEQ ID No: 157 and a light chain constant region comprising or consisting of SEQ ID No: 158.

1-G5 L1 (hIgG1K)

Alternatively, in another embodiment, the humanised antibody or antigen-binding fragment thereof is referred to herein as 1-G5_L1 (hIgG1K).

Preferably, the antibody or antigen-binding fragment thereof comprises a heavy chain variable (VH) region comprising or consisting of a sequence as substantially set out in SEQ ID No: 161, or a variant or fragment thereof.

Preferably, the antibody or antigen-binding fragment thereof comprises a light chain variable (VL) region comprising or consisting of a sequence as substantially set out in SEQ ID No: 193, or a variant or fragment thereof.

Preferably, the antibody or antigen-binding fragment thereof comprises a heavy chain variable region comprising or consisting of SEQ ID No: 161 and a light chain variable region comprising or consisting of SEQ ID No: 193.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-G5 H2 L1

Alternatively, in another embodiment, the humanised antibody or antigen-binding fragment thereof is referred to herein as 1-G5_H2_L1.

Preferably, the antibody or antigen-binding fragment thereof comprises a heavy chain variable (VH) region comprising or consisting of a sequence as substantially set out in SEQ ID No: 184, or a variant or fragment thereof.

Preferably, the antibody or antigen-binding fragment thereof comprises a light chain variable (VL) region comprising or consisting of a sequence as substantially set out in SEQ ID No: 193, or a variant or fragment thereof.

Preferably, the antibody or antigen-binding fragment thereof comprises a heavy chain variable region comprising or consisting of SEQ ID No: 184 and a light chain variable region comprising or consisting of SEQ ID No: 193.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-G5 H2 L0

Alternatively, in another embodiment, the humanised antibody or antigen-binding fragment thereof is referred to herein as 1-G5_H2_L0.

Preferably, the antibody or antigen-binding fragment thereof comprises a heavy chain variable (VH) region comprising or consisting of a sequence as substantially set out in SEQ ID No: 184, or a variant or fragment thereof.

Preferably, the antibody or antigen-binding fragment thereof comprises a light chain variable (VL) region comprising or consisting of a sequence as substantially set out in SEQ ID No: 192, or a variant or fragment thereof.

Preferably, the antibody or antigen-binding fragment thereof comprises a heavy chain variable region comprising or consisting of SEQ ID No: 184 and a light chain variable region comprising or consisting of SEQ ID No: 192.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-G5 H1 L1

Alternatively, in another embodiment, the humanised antibody or antigen-binding fragment thereof is referred to herein as 1-G5_H1_L1.

Preferably, the antibody or antigen-binding fragment thereof comprises a heavy chain variable (VH) region comprising or consisting of a sequence as substantially set out in SEQ ID No: 183, or a variant or fragment thereof.

Preferably, the antibody or antigen-binding fragment thereof comprises a light chain variable (VL) region comprising or consisting of a sequence as substantially set out in SEQ ID No: 193, or a variant or fragment thereof.

Preferably, the antibody or antigen-binding fragment thereof comprises a heavy chain variable region comprising or consisting of SEQ ID No: 183 and a light chain variable region comprising or consisting of SEQ ID No: 193.

Preferably, the antibody or antigen-binding fragment thereof comprises a heavy chain constant (HC) region comprising or consisting of a sequence as substantially set out in

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-G5 H1 L0

Alternatively, in another embodiment, the humanised antibody or antigen-binding fragment thereof is referred to herein as 1-G5_H1_L1.

Preferably, the antibody or antigen-binding fragment thereof comprises a heavy chain variable (VH) region comprising or consisting of a sequence as substantially set out in SEQ ID No: 183, or a variant or fragment thereof.

Preferably, the antibody or antigen-binding fragment thereof comprises a light chain variable (VL) region comprising or consisting of a sequence as substantially set out in SEQ ID No: 192, or a variant or fragment thereof.

Preferably, the antibody or antigen-binding fragment thereof comprises a heavy chain variable region comprising or consisting of SEQ ID No: 183 and a light chain variable region comprising or consisting of SEQ ID No: 192.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain

### 1-G5 H0 L1

Alternatively, in another embodiment, the humanised antibody or antigen-binding fragment thereof is referred to herein as 1-G5_H0_L1.

Preferably, the antibody or antigen-binding fragment thereof comprises a CDR-H1 domain comprising or consisting of a sequence as substantially set out in SEQ ID No: 181, or a variant or fragment thereof. Preferably, the antibody or antigen-binding fragment thereof comprises a CDR-H2 domain comprising or consisting of a sequence as substantially set out in SEQ ID No: 82, or a variant or fragment thereof. Preferably, the antibody or antigen-binding fragment thereof comprises a CDR-H3 domain comprising or consisting of a sequence as substantially set out in SEQ ID No: 83, or a variant or fragment thereof.

Preferably, the antibody or antigen-binding fragment thereof comprises a CDR-L1 domain comprising or consisting of a sequence as substantially set out in SEQ ID No: 90, or a variant or fragment thereof. Preferably, the antibody or antigen-binding fragment thereof comprises a CDR-L2 domain comprising or consisting of a sequence as substantially set out in SEQ ID No: 91, or a variant or fragment thereof. Preferably, the antibody or antigen-binding fragment thereof comprises a CDR-L3 domain comprising or consisting of a sequence as substantially set out in SEQ ID No: 92, or a variant or fragment thereof.

Preferably, the antibody or antigen-binding fragment thereof comprises a CDR-H1 domain comprising or consisting of SEQ ID No: 181, a CDR-H2 domain comprising or consisting of SEQ ID No: 82; a CDR-H3 domain comprising or consisting of SEQ ID No: 83, a CDR-L1 domain comprising or consisting of SEQ ID No: 90, a CDR-L2 domain comprising or consisting of SEQ ID No: 91, and a CDR-L3 domain comprising or consisting of SEQ ID No: 92.

Preferably, the antibody or antigen-binding fragment thereof comprises a heavy chain variable (VH) region comprising or consisting of a sequence as substantially set out in SEQ ID No: 182, or a variant or fragment thereof.

Preferably, the antibody or antigen-binding fragment thereof comprises a light chain variable (VL) region comprising or consisting of a sequence as substantially set out in SEQ ID No: 193, or a variant or fragment thereof.

Preferably, the antibody or antigen-binding fragment thereof comprises a heavy chain variable region comprising or consisting of SEQ ID No: 182 and a light chain variable region comprising or consisting of SEQ ID No: 193.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain constant region comprising or consisting of SEQ ID No: 157 and a light chain constant region comprising or consisting of SEQ ID No: 158.

### 1-G5 H0 L0

Alternatively, in another embodiment, the humanised antibody or antigen-binding fragment thereof is referred to herein as 1-G5_H0_L0.

Preferably, the antibody or antigen-binding fragment thereof comprises a CDR-H1 domain comprising or consisting of a sequence as substantially set out in SEQ ID No: 181, or a variant or fragment thereof. Preferably, the antibody or antigen-binding fragment thereof comprises a CDR-H2 domain comprising or consisting of a sequence as substantially set out in SEQ ID No: 82, or a variant or fragment thereof. Preferably, the antibody or antigen-binding fragment thereof comprises a CDR-H3 domain comprising or consisting of a sequence as substantially set out in SEQ ID No: 83, or a variant or fragment thereof.

Preferably, the antibody or antigen-binding fragment thereof comprises a CDR-L1 domain comprising or consisting of a sequence as substantially set out in SEQ ID No: 90, or a variant or fragment thereof. Preferably, the antibody or antigen-binding fragment thereof comprises a CDR-L2 domain comprising or consisting of a sequence as substantially set out in SEQ ID No: 91, or a variant or fragment thereof. Preferably, the antibody or antigen-binding fragment thereof comprises a CDR-L3 domain comprising or consisting of a sequence as substantially set out in SEQ ID No: 92, or a variant or fragment thereof.

Preferably, the antibody or antigen-binding fragment thereof comprises a CDR-H1 domain comprising or consisting of SEQ ID No: 181, a CDR-H2 domain comprising or consisting of SEQ ID No: 82; a CDR-H3 domain comprising or consisting of SEQ ID

No: 83, a CDR-L1 domain comprising or consisting of SEQ ID No: 90, a CDR-L2 domain comprising or consisting of SEQ ID No: 91, and a CDR-L3 domain comprising or consisting of SEQ ID No: 92.

Preferably, the antibody or antigen-binding fragment thereof comprises a heavy chain variable (VH) region comprising or consisting of a sequence as substantially set out in SEQ ID No: 182, or a variant or fragment thereof.

Preferably, the antibody or antigen-binding fragment thereof comprises a light chain variable (VL) region comprising or consisting of a sequence as substantially set out in SEQ ID No: 192, or a variant or fragment thereof.

Preferably, the antibody or antigen-binding fragment thereof comprises a heavy chain variable region comprising or consisting of SEQ ID No: 182 and a light chain variable region comprising or consisting of SEQ ID No: 192.

Preferably, the antibody or antigen-binding fragment thereof comprises a heavy chain constant (HC) region comprising or consisting of a sequence as substantially set out in SEQ ID No: 157, or a variant or fragment thereof.

Preferably, the antibody or antigen-binding fragment thereof comprises a light chain constant (LC) region comprising or consisting of a sequence as substantially set out in SEQ ID No: 158, or a variant or fragment thereof.

Preferably, the antibody or antigen-binding fragment thereof comprises a heavy chain

### 4-H9

In one embodiment, the antibody or antigen-binding fragment thereof is referred to herein as 4-H9. The antibody or antigen-binding fragment thereof may comprise a CDR-H1 domain of SEQ ID No: 99, which is provided herein, as follows:

[SEQ ID No: 99]
GFSLNSFA

Thus, preferably, the antibody or antigen-binding fragment thereof comprises a CDR-H1 domain comprising or consisting of a sequence as substantially set out in SEQ ID No: 99, or a variant or fragment thereof.

The antibody or antigen-binding fragment thereof may comprise a CDR-H2 domain of SEQ ID No: 100, which is provided herein, as follows:

[SEQ ID No: 100]
ITVDGHT

Thus, preferably the antibody or antigen-binding fragment thereof comprises a CDR-H2 domain comprising or consisting of a sequence as substantially set out in SEQ ID No: 100, or a variant or fragment thereof.

The antibody or antigen-binding fragment thereof may comprise a CDR-H3 domain of SEQ ID No: 101, which is provided herein, as follows:

[SEQ ID No: 101]
AREDAGDAGYIYATYNI

Thus, preferably the antibody or antigen-binding fragment thereof comprises a CDR-H3 domain comprising or consisting of a sequence as substantially set out in SEQ ID No: 101, or a variant or fragment thereof.

Preferably, the antibody or antigen-binding fragment thereof comprises a CDR-H1 domain comprising or consisting of SEQ ID No: 99, a CDR-H2 domain comprising or consisting of SEQ ID No: 100 and/or a CDR-H3 domain comprising or consisting of SEQ ID No: 101. Preferably, however, the antibody or antigen-binding fragment thereof comprises a CDR-H1 domain comprising or consisting of SEQ ID No: 99, a CDR-H2 domain comprising or consisting of SEQ ID No: 100 and a CDR-H3 domain comprising or consisting of SEQ ID No: 101.

The antibody or antigen-binding fragment thereof may comprise a FR-H1 domain of SEQ ID No: 102, which is provided herein, as follows:

[SEQ ID No: 102]
SQSVEESGGRLVTPGTPLTLTCTAS

Thus, preferably, the antibody or antigen-binding fragment thereof comprises a FR-H1 domain comprising or consisting of a sequence as substantially set out in SEQ ID No: 102, or a variant or fragment thereof.

The antibody or antigen-binding fragment thereof may comprise a FR-H2 domain of SEQ ID No: 103, which is provided herein, as follows:

[SEQ ID No: 103]
MSWVRQAPGKGLEWIGI

Thus, preferably, the antibody or antigen-binding fragment thereof comprises a FR-H2 domain comprising or consisting of a sequence as substantially set out in SEQ ID No: 103, or a variant or fragment thereof.

The antibody or antigen-binding fragment thereof may comprise a FR-H3 domain of SEQ ID No: 104, which is provided herein, as follows:

[SEQ ID No: 104]
YYASWAKGRFTISKASTTVDLKITSPTTEDTATYFC

Thus, preferably, the antibody or antigen-binding fragment thereof comprises a FR-H3 domain comprising or consisting of a sequence as substantially set out in SEQ ID No: 104, or a variant or fragment thereof.

The antibody or antigen-binding fragment thereof may comprise a FR-H4 domain of SEQ ID No: 105, which is provided herein, as follows:

[SEQ ID No: 105]
WGPGTLVTVSS

Thus, preferably, the antibody or antigen-binding fragment thereof comprises a FR-H4 domain comprising or consisting of a sequence as substantially set out in SEQ ID No: 105, or a variant or fragment thereof.

Preferably, the antibody or antigen-binding fragment thereof comprises a FR-H1 domain comprising or consisting of SEQ ID No: 102, a FR-H2 domain comprising or consisting of SEQ ID No: 103, a FR-H3 domain comprising or consisting of SEQ ID No: 104, and/or a FR-H4 domain comprising or consisting of SEQ ID No: 105. Preferably, however, the antibody or antigen-binding fragment thereof comprises a FR-H1 domain comprising or consisting of SEQ ID No: 102, a FR-H2 domain comprising or consisting of SEQ ID No: 103, a FR-H3 domain comprising or consisting of SEQ ID No: 104, and a FR-H4 domain comprising or consisting of SEQ ID No: 105.

The antibody or antigen-binding fragment thereof may comprise a heavy chain variable (VH) sequence as set out in SEQ ID No: 106, which is provided herein, as follows:

[SEQ ID No: 106]
QSVEESGGRLVTPGTPLTLTCTASGFSLNSFAMSWVRQAPGKGLEWIGI
ITVDGHTYYASWAKGRFTISKASTTVDLKITSPTTEDTATYFCAREDAG
DAGYIYATYNIWGPGTLVTVSS

Preferably, the antibody or antigen-binding fragment thereof comprises a heavy chain variable (VH) region comprising or consisting of a sequence as substantially set out in SEQ ID No: 106, or a variant or fragment thereof.

One embodiment of the nucleotide sequence encoding the heavy chain variable (VH) region is referred to herein as SEQ ID No: 107, as follows:

[SEQ ID No: 107]
CAATCGGTGGAGGAGTCCGGGGGTCGCCTGGTCACGCCTGGGACACCCC
TGACACTCACCTGCACAGCCTCTGGATTCTCCCTCAATAGCTTTGCGAT
GAGCTGGGTCCGCCAGGCTCCAGGGAAGGGGCTGGAATGGATCGGAATC
ATTACTGTTGATGGTCACACATACTACGCGAGCTGGGCGAAAGGCCGAT
TCACCATCTCCAAAGCCTCGACCACGGTGGATCTGAAAATCACCAGTCC
GACAACCGAGGACACGGCCACCTATTTCTGTGCCAGAGAGGATGCTGGT
GATGCTGGTTATATTTATGCTACCTATAACATCTGGGGCCCAGGGACCC
TCGTCACCGTCTCTTCA

Preferably, the antibody or antigen-binding fragment thereof comprises a heavy chain variable (VH) region encoded by a nucleic acid sequence as substantially set out in SEQ ID No: 107, or a variant or fragment thereof.

The antibody or antigen-binding fragment thereof may comprise a CDR-L1 domain of SEQ ID No: 108, which is provided herein, as follows:

[SEQ ID No: 108]
EDIGYG

Thus, preferably, the antibody or antigen-binding fragment thereof comprises a CDR-L1 domain comprising or consisting of a sequence as substantially set out in SEQ ID No: 108, or a variant or fragment thereof.

The antibody or antigen-binding fragment thereof may comprise a CDR-L2 domain of SEQ ID No: 109, which is provided herein, as follows:

[SEQ ID No: 109]
GAN

Thus, preferably, the antibody or antigen-binding fragment thereof comprises a CDR-L2 domain comprising or consisting of a sequence as substantially set out in SEQ ID No: 109, or a variant or fragment thereof.

The antibody or antigen-binding fragment thereof may comprise a CDR-L3 domain of SEQ ID No: 110, which is provided herein, as follows:

[SEQ ID No: 110]
QQGYSTPPT

Thus, preferably, the antibody or antigen-binding fragment thereof comprises a CDR-L3 domain comprising or consisting of a sequence as substantially set out in SEQ ID No: 110, or a variant or fragment thereof.

Preferably, the antibody or antigen-binding fragment thereof comprises a CDR-L1 domain comprising or consisting of SEQ ID No: 108, a CDR-L2 domain comprising or consisting of SEQ ID No: 109, and/or a CDR-L3 domain comprising or consisting of SEQ ID No: 110. However, preferably the antibody or antigen-binding fragment thereof comprises a CDR-L1 domain comprising or consisting of SEQ ID No: 108, a CDR-L2 domain comprising or consisting of SEQ ID No: 109, and a CDR-L3 domain comprising or consisting of SEQ ID No: 110.

The antibody or antigen-binding fragment thereof may comprise a FR-L1 domain of SEQ ID No: 111, which is provided herein, as follows:

[SEQ ID No: 111]
AIEMTQTPSSLAASVGDTVTITCKAS

Thus, preferably, the antibody or antigen-binding fragment thereof comprises a FR-L1 domain comprising or consisting of a sequence as substantially set out in SEQ ID No: 111, or a variant or fragment thereof.

The antibody or antigen-binding fragment thereof may comprise a FR-L2 domain of SEQ ID No: 112, which is provided herein, as follows:

[SEQ ID No: 112]
LAWYQQKLGIAPKLLIY

Thus, preferably, the antibody or antigen-binding fragment thereof comprises a FR-L2 domain comprising or consisting of a sequence as substantially set out in SEQ ID No: 112, or a variant or fragment thereof.

The antibody or antigen-binding fragment thereof may comprise a FR-L3 domain of SEQ ID No: 113, which is provided herein, as follows:

[SEQ ID No: 113]
TLESGVPSRFSGSGSETDYTLTISSVQAEDAGIYYC

Thus, preferably, the antibody or antigen-binding fragment thereof comprises a FR-L3 domain comprising or consisting of a sequence as substantially set out in SEQ ID No: 113, or a variant or fragment thereof.

The antibody or antigen-binding fragment thereof may comprise a FR-L4 domain of SEQ ID No: 114, which is provided herein, as follows:

[SEQ ID No: 114]
FGAGTMVEIQ

Thus, preferably, the antibody or antigen-binding fragment thereof comprises a FR-L4 domain comprising or consisting of a sequence as substantially set out in SEQ ID No: 114, or a variant or fragment thereof.

Preferably, the antibody or antigen-binding fragment thereof comprises a FR-L1 domain comprising or consisting of SEQ ID No: 111, a FR-L2 domain comprising or consisting of SEQ ID No: 112, a FR-L3 domain comprising or consisting of SEQ ID No: 113, and/or a FR-L4 domain comprising or consisting of SEQ ID No: 114. Preferably, however, the antibody or antigen-binding fragment thereof comprises a FR-L1 domain comprising or consisting of SEQ ID No: 111, a FR-L2 domain comprising or consisting of SEQ ID No: 112, a FR-L3 domain comprising or consisting of SEQ ID No: 113, and a FR-L4 domain comprising or consisting of SEQ ID No: 114.

The antibody or antigen-binding fragment thereof may comprise a light chain variable (VL) sequence as set out in SEQ ID No: 115, which is provided herein, as follows:

[SEQ ID No: 115]
AIEMTQTPSSLAASVGDTVTITCKASEDIGYGLAWYQQKLGIAPK
LLIYGANTLESGVPSRFSGSGSETDYTLTISSVQAEDAGIYYCQQ
GYSTPPTFGAGTMVEIQ

Preferably, the antibody or antigen-binding fragment thereof comprises a light chain variable region comprising or consisting of a sequence as substantially set out in SEQ ID No: 115, or a variant or fragment thereof.

One embodiment of the nucleotide sequence encoding the light chain variable (VL) region is referred to herein as SEQ ID No: 116, as follows:

[SEQ ID No: 116]
GCGATTGAAATGACCCAGACTCCATCCTCCCTGGCTGCATCTGTG
GGAGACACAGTCACCATCACTTGTAAGGCCAGTGAGGACATTGGT
TATGGGTTAGCCTGGTATCAGCAGAAACTGGGGATAGCTCCTAAG
CTCCTGATCTATGGGGCAAACACTTTAGAATCTGGGGTCCCATCG
AGGTTCAGTGGCAGCGGATCAGAGACCGATTACACCCTCACCATC
AGCAGCGTGCAGGCTGAAGATGCAGGAATTTATTACTGTCAGCAA
GGATATAGTACCCCTCCTACTTTCGGTGCGGGGACCATGGTGGAG
ATCCAA

Preferably, the antibody or antigen-binding fragment thereof comprises a light chain variable (VL) region encoded by a nucleic acid sequence as substantially set out in SEQ ID No: 116, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 99, a CDR-H2 domain comprising or consisting of SEQ ID No: 100; a CDR-H3 domain comprising or consisting of SEQ ID

No: 101, a CDR-L1 domain comprising or consisting of SEQ ID No: 108, a CDR-L2 domain comprising or consisting of SEQ ID No: 109, and a CDR-L3 domain comprising or consisting of SEQ ID No: 110.

Preferably, the antibody or antigen-binding fragment thereof comprises a heavy chain variable region comprising or consisting of SEQ ID No: 106, and a light chain variable region comprising or consisting of SEQ ID No: 115.

Preferably, the antibody or antigen-binding fragment thereof comprises a heavy chain variable region encoded by a nucleic acid sequence comprising or consisting of SEQ ID

No: 107, and a light chain variable region encoded by a nucleic acid sequence comprising or consisting of SEQ ID No: 116.

### 4-B12

In one embodiment, the antibody or antigen-binding fragment thereof is referred to herein as 4-B12. The antibody or antigen-binding fragment thereof may comprise a CDR-H1 domain of SEQ ID No: 117, which is provided herein, as follows:

[SEQ ID No: 117]
GFSLNSFA

Thus, preferably, the antibody or antigen-binding fragment thereof comprises a CDR-H1 domain comprising or consisting of a sequence as substantially set out in SEQ ID No: 117, or a variant or fragment thereof.

The antibody or antigen-binding fragment thereof may comprise a CDR-H2 domain of SEQ ID No: 118, which is provided herein, as follows:

[SEQ ID No: 118]
ITVDGHT

Thus, preferably the antibody or antigen-binding fragment thereof comprises a CDR-H2 domain comprising or consisting of a sequence as substantially set out in SEQ ID No: 118, or a variant or fragment thereof.

The antibody or antigen-binding fragment thereof may comprise a CDR-H3 domain of SEQ ID No: 119, which is provided herein, as follows:

[SEQ ID No: 119]
AREDAGDAGYIYATYNI

Thus, preferably the antibody or antigen-binding fragment thereof comprises a CDR-H3 domain comprising or consisting of a sequence as substantially set out in SEQ ID No: 119, or a variant or fragment thereof.

Preferably, the antibody or antigen-binding fragment thereof comprises a CDR-H1 domain comprising or consisting of SEQ ID No: 117, a CDR-H2 domain comprising or consisting of SEQ ID No: 118 and/or a CDR-H3 domain comprising or consisting of

SEQ ID No: 119. Preferably, however, the antibody or antigen-binding fragment thereof comprises a CDR-H1 domain comprising or consisting of SEQ ID No: 117, a CDR-H2 domain comprising or consisting of SEQ ID No: 118 and a CDR-H3 domain comprising or consisting of SEQ ID No: 119.

The antibody or antigen-binding fragment thereof may comprise a FR-H1 domain of SEQ ID No: 120, which is provided herein, as follows:

[SEQ ID No: 120]
SQSVKESEGRLVTPGTPLTLTCTVS

Thus, preferably, the antibody or antigen-binding fragment thereof comprises a FR-H1 domain comprising or consisting of a sequence as substantially set out in SEQ ID No: 120, or a variant or fragment thereof.

The antibody or antigen-binding fragment thereof may comprise a FR-H2 domain of SEQ ID No: 121, which is provided herein, as follows:

[SEQ ID No: 121]
MSWVRQAPGKGLEWIGI

Thus, preferably, the antibody or antigen-binding fragment thereof comprises a FR-H2 domain comprising or consisting of a sequence as substantially set out in SEQ ID No: 121, or a variant or fragment thereof.

The antibody or antigen-binding fragment thereof may comprise a FR-H3 domain of SEQ ID No: 122, which is provided herein, as follows:

[SEQ ID No: 122]
YYANWAKDRFTISKASTTVDLKITSPTTEDTATYFC

Thus, preferably, the antibody or antigen-binding fragment thereof comprises a FR-H3 domain comprising or consisting of a sequence as substantially set out in SEQ ID No: 122, or a variant or fragment thereof.

The antibody or antigen-binding fragment thereof may comprise a FR-H4 domain of SEQ ID No: 123, which is provided herein, as follows:

[SEQ ID No: 123]
WGPGTLVTVSS

Thus, preferably, the antibody or antigen-binding fragment thereof comprises a FR-H4 domain comprising or consisting of a sequence as substantially set out in SEQ ID No: 123, or a variant or fragment thereof.

Preferably, the antibody or antigen-binding fragment thereof comprises a FR-H1 domain comprising or consisting of SEQ ID No: 120, a FR-H2 domain comprising or consisting of SEQ ID No: 121, a FR-H3 domain comprising or consisting of SEQ ID No: 122, and/or a FR-H4 domain comprising or consisting of SEQ ID No: 123. Preferably, however, the antibody or antigen-binding fragment thereof comprises a FR-H1 domain comprising or consisting of SEQ ID No: 120, a FR-H2 domain comprising or consisting of SEQ ID No: 121, a FR-H3 domain comprising or consisting of SEQ ID No: 122, and a FR-H4 domain comprising or consisting of SEQ ID No: 123.

The antibody or antigen-binding fragment thereof may comprise a heavy chain variable (VH) sequence as set out in SEQ ID No: 124, which is provided herein, as follows:

[SEQ ID No: 124]
QSVKESEGRLVTPGTPLTLTCTVSGFSLNSFAMSWVRQAPGKGLE
WIGIITVDGHTYYANWAKDRFTISKASTTVDLKITSPTTEDTATY
FCAREDAGDAGYIYATYNIWGPGTLVTVSS

Preferably, the antibody or antigen-binding fragment thereof comprises a heavy chain variable (VH) region comprising or consisting of a sequence as substantially set out in SEQ ID No: 124, or a variant or fragment thereof.

One embodiment of the nucleotide sequence encoding the heavy chain variable (VH) region is referred to herein as SEQ ID No: 125, as follows:

[SEQ ID No: 125]
CAGTCGGTGAAGGAGTCCGAGGGTCGCCTGGTCACGCCTGGGACA
CCCCTGACACTCACCTGCACAGTCTCTGGATTCTCCCTCAATAGC
TTTGCGATGAGCTGGGTCCGCCAGGCTCCAGGGAAGGGGCTGGAA
TGGATCGGAATCATAACTGTTGATGGTCACACATACTACGCGAAC
TGGGCGAAAGACCGATTCACCATCTCCAAAGCCTCGACCACGGTG
GATCTGAAAATCACCAGTCCGACAACCGAGGACACGGCCACCTAT
TTCTGTGCCAGAGAGGATGCTGGTGATGCTGGTTATATTTATGCT
ACCTATAACATCTGGGGCCCGGGCACCCTGGTCACCGTCTCCTCA

Preferably, the antibody or antigen-binding fragment thereof comprises a heavy chain variable (VH) region encoded by a nucleic acid sequence as substantially set out in SEQ ID No: 125, or a variant or fragment thereof.

The antibody or antigen-binding fragment thereof may comprise a CDR-L1 domain of SEQ ID No: 126, which is provided herein, as follows:

[SEQ ID No: 126]
EDIGYG

Thus, preferably, the antibody or antigen-binding fragment thereof comprises a CDR-L1 domain comprising or consisting of a sequence as substantially set out in SEQ ID No: 126, or a variant or fragment thereof.

The antibody or antigen-binding fragment thereof may comprise a CDR-L2 domain of SEQ ID No: 127, which is provided herein, as follows:

[SEQ ID No: 127]
GAN

Thus, preferably, the antibody or antigen-binding fragment thereof comprises a CDR-L2 domain comprising or consisting of a sequence as substantially set out in SEQ ID No: 127, or a variant or fragment thereof.

The antibody or antigen-binding fragment thereof may comprise a CDR-L3 domain of SEQ ID No: 128, which is provided herein, as follows:

[SEQ ID No: 128]
QQGYSTPPT

Thus, preferably, the antibody or antigen-binding fragment thereof comprises a CDR-L3 domain comprising or consisting of a sequence as substantially set out in SEQ ID No: 128, or a variant or fragment thereof.

Preferably, the antibody or antigen-binding fragment thereof comprises a CDR-L1 domain comprising or consisting of SEQ ID No: 126, a CDR-L2 domain comprising or consisting of SEQ ID No: 127, and/or a CDR-L3 domain comprising or consisting of

SEQ ID No: 128. However, preferably the antibody or antigen-binding fragment thereof comprises a CDR-L1 domain comprising or consisting of SEQ ID No: 126, a CDR-L2 domain comprising or consisting of SEQ ID No: 127, and a CDR-L3 domain comprising or consisting of SEQ ID No: 128.

The antibody or antigen-binding fragment thereof may comprise a FR-L1 domain of SEQ ID No: 129, which is provided herein, as follows:

[SEQ ID No: 129]
DPVLTQTASSLAASVGDTVTITCKAS

Thus, preferably, the antibody or antigen-binding fragment thereof comprises a FR-L1 domain comprising or consisting of a sequence as substantially set out in SEQ ID No: 129, or a variant or fragment thereof.

The antibody or antigen-binding fragment thereof may comprise a FR-L2 domain of SEQ ID No: 130, which is provided herein, as follows:

[SEQ ID No: 130]
LAWYQQKPGQPPKLLIY

Thus, preferably, the antibody or antigen-binding fragment thereof comprises a FR-L2 domain comprising or consisting of a sequence as substantially set out in SEQ ID No: 130, or a variant or fragment thereof.

The antibody or antigen-binding fragment thereof may comprise a FR-L3 domain of SEQ ID No: 131, which is provided herein, as follows:

[SEQ ID No: 131]
TLESGVPSRFTGSGSETDYTLTISSVQAEDAGIYYC

Thus, preferably, the antibody or antigen-binding fragment thereof comprises a FR-L3 domain comprising or consisting of a sequence as substantially set out in SEQ ID No: 131, or a variant or fragment thereof.

The antibody or antigen-binding fragment thereof may comprise a FR-L4 domain of SEQ ID No: 132, which is provided herein, as follows:

[SEQ ID No: 132]
FGAGTKVEIK

Thus, preferably, the antibody or antigen-binding fragment thereof comprises a FR-L4 domain comprising or consisting of a sequence as substantially set out in SEQ ID No: 132, or a variant or fragment thereof.

Preferably, the antibody or antigen-binding fragment thereof comprises a FR-L1 domain comprising or consisting of SEQ ID No: 129, a FR-L2 domain comprising or consisting of SEQ ID No: 130, a FR-L3 domain comprising or consisting of SEQ ID No:

131, and/or a FR-L4 domain comprising or consisting of SEQ ID No: 132. Preferably, however, the antibody or antigen-binding fragment thereof comprises a FR-L1 domain comprising or consisting of SEQ ID No: 129, a FR-L2 domain comprising or consisting of SEQ ID No: 130, a FR-L3 domain comprising or consisting of SEQ ID No: 131, and a FR-L4 domain comprising or consisting of SEQ ID No: 132.

The antibody or antigen-binding fragment thereof may comprise a light chain variable (VL) sequence as set out in SEQ ID No: 133, which is provided herein, as follows:

[SEQ ID No: 133]
DPVLTQTASSLAASVGDTVTITCKASEDIGYGLAWYQQKPGQPPK
LLIYGANTLESGVPSRFTGSGSETDYTLTISSVQAEDAGIYYCQQ
GYSTPPTFGAGTKVEIK

Preferably, the antibody or antigen-binding fragment thereof comprises a light chain variable region comprising or consisting of a sequence as substantially set out in SEQ ID No: 133, or a variant or fragment thereof.

One embodiment of the nucleotide sequence encoding the light chain variable (VL) region is referred to herein as SEQ ID No: 134, as follows:

[SEQ ID No: 134]
GATCCTGTGCTGACCCAGACTGCGTCCTCCCTGGCTGCATCTGTG
GGAGACACAGTCACCATCACTTGTAAGGCCAGTGAGGACATTGGT
TATGGGTTAGCCTGGTATCAGCAGAAACCAGGGCAGCCTCCCAAG
CTCCTGATCTATGGGGCAAACACTTTAGAATCTGGGGTCCCATCG
AGGTTCACTGGCAGCGGATCAGAGACCGATTACACCCTCACCATC
AGCAGCGTGCAGGCTGAAGATGCAGGAATTTATTACTGTCAGCAA
GGATATAGTACCCCTCCTACTTTCGGTGCGGGCACCAAGGTAGAA
ATCAAA

Preferably, the antibody or antigen-binding fragment thereof comprises a light chain variable (VL) region encoded by a nucleic acid sequence as substantially set out in SEQ ID No: 134, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 117, a CDR-H2 domain comprising or consisting of SEQ ID No: 118; a CDR-H3 domain comprising or consisting of SEQ ID

No: 119, a CDR-L1 domain comprising or consisting of SEQ ID No: 126, a CDR-L2 domain comprising or consisting of SEQ ID No: 127, and a CDR-L3 domain comprising or consisting of SEQ ID No: 128.

Preferably, the antibody or antigen-binding fragment thereof comprises a heavy chain variable region comprising or consisting of SEQ ID No: 124, and a light chain variable region comprising or consisting of SEQ ID No: 133.

Preferably, the antibody or antigen-binding fragment thereof comprises a heavy chain variable region encoded by a nucleic acid sequence comprising or consisting of SEQ ID

No: 125, and a light chain variable region encoded by a nucleic acid sequence comprising or consisting of SEQ ID No: 134.

### 4-C6

In one embodiment, the antibody or antigen-binding fragment thereof is referred to herein as 4-C6. The antibody or antigen-binding fragment thereof may comprise a CDR-H1 domain of SEQ ID No: 135, which is provided herein, as follows:

[SEQ ID No: 135]
GFSLNTYV

Thus, preferably, the antibody or antigen-binding fragment thereof comprises a CDR-H1 domain comprising or consisting of a sequence as substantially set out in SEQ ID No: 135, or a variant or fragment thereof.

The antibody or antigen-binding fragment thereof may comprise a CDR-H2 domain of SEQ ID No: 136, which is provided herein, as follows:

[SEQ ID No: 136]
INGDSNT

Thus, preferably the antibody or antigen-binding fragment thereof comprises a CDR-H2 domain comprising or consisting of a sequence as substantially set out in SEQ ID No: 136, or a variant or fragment thereof.

The antibody or antigen-binding fragment thereof may comprise a CDR-H3 domain of SEQ ID No: 137, which is provided herein, as follows:

[SEQ ID No: 137]
AREDAADAGYVYATYNI

Thus, preferably the antibody or antigen-binding fragment thereof comprises a CDR-H3 domain comprising or consisting of a sequence as substantially set out in SEQ ID No: 137, or a variant or fragment thereof.

Preferably, the antibody or antigen-binding fragment thereof comprises a CDR-H1 domain comprising or consisting of SEQ ID No: 135, a CDR-H2 domain comprising or consisting of SEQ ID No: 136 and/or a CDR-H3 domain comprising or consisting of SEQ ID No: 137. Preferably, however, the antibody or antigen-binding fragment thereof comprises a CDR-H1 domain comprising or consisting of SEQ ID No: 135, a CDR-H2 domain comprising or consisting of SEQ ID No: 136 and a CDR-H3 domain comprising or consisting of SEQ ID No: 137.

The antibody or antigen-binding fragment thereof may comprise a FR-H1 domain of SEQ ID No: 138, which is provided herein, as follows:

[SEQ ID No: 138]
SQSLEESGGRLVTPGTPLTLTCTAS

Thus, preferably, the antibody or antigen-binding fragment thereof comprises a FR-H1 domain comprising or consisting of a sequence as substantially set out in SEQ ID No: 138, or a variant or fragment thereof.

The antibody or antigen-binding fragment thereof may comprise a FR-H2 domain of SEQ ID No: 139, which is provided herein, as follows:

[SEQ ID No: 139]
MTWVRQAPGKGLEWIGF

Thus, preferably, the antibody or antigen-binding fragment thereof comprises a FR-H2 domain comprising or consisting of a sequence as substantially set out in SEQ ID No: 139, or a variant or fragment thereof.

The antibody or antigen-binding fragment thereof may comprise a FR-H3 domain of SEQ ID No: 140, which is provided herein, as follows:

[SEQ ID No: 140]
YYANWAKGRFTISKTSTTVDLKITSPTTEDTATYFC

Thus, preferably, the antibody or antigen-binding fragment thereof comprises a FR-H3 domain comprising or consisting of a sequence as substantially set out in SEQ ID No: 140, or a variant or fragment thereof.

The antibody or antigen-binding fragment thereof may comprise a FR-H4 domain of SEQ ID No: 141, which is provided herein, as follows:

[SEQ ID No: 141]
WGTGTLVTISS

Thus, preferably, the antibody or antigen-binding fragment thereof comprises a FR-H4 domain comprising or consisting of a sequence as substantially set out in SEQ ID No: 141, or a variant or fragment thereof.

Preferably, the antibody or antigen-binding fragment thereof comprises a FR-H1 domain comprising or consisting of SEQ ID No: 138, a FR-H2 domain comprising or consisting of SEQ ID No: 139, a FR-H3 domain comprising or consisting of SEQ ID No: 140, and/or a FR-H4 domain comprising or consisting of SEQ ID No: 141. Preferably, however, the antibody or antigen-binding fragment thereof comprises a FR-H1 domain comprising or consisting of SEQ ID No: 138, a FR-H2 domain comprising or consisting of SEQ ID No: 139, a FR-H3 domain comprising or consisting of SEQ ID No: 140, and a FR-H4 domain comprising or consisting of SEQ ID No: 141.

The antibody or antigen-binding fragment thereof may comprise a heavy chain variable (VH) sequence as set out in SEQ ID No: 142, which is provided herein, as follows:

[SEQ ID No: 142]
QSLEESGGRLVTPGTPLTLTCTASGFSLNTYVMTWVRQAPGKGLEWIGF
INGDSNTYYANWAKGRFTISKTSTTVDLKITSPTTEDTATYFCAREDAA
DAGYVYATYNIWGTGTLVTISS

Preferably, the antibody or antigen-binding fragment thereof comprises a heavy chain variable (VH) region comprising or consisting of a sequence as substantially set out in SEQ ID No: 142, or a variant or fragment thereof.

One embodiment of the nucleotide sequence encoding the heavy chain variable (VH) region is referred to herein as SEQ ID No: 143, as follows:

[SEQ ID No: 143]
CAGTCGCTGGAGGAGTCCGGGGGTCGCCTGGTCACGCCTGGGACACCCC
TGACACTCACCTGCACAGCCTCTGGATTCTCCCTCAATACCTATGTAAT
GACCTGGGTCCGCCAGGCTCCAGGGAAGGGGCTGGAATGGATCGGATTC
ATTAATGGTGATAGTAACACATACTACGCGAACTGGGCGAAAGGCCGAT
TCACCATCTCCAAAACCTCGACCACGGTGGATCTGAAAATCACCAGTCC
GACAACCGAGGACACGGCCACCTATTTCTGTGCCAGAGAGGATGCTGCT
GATGCTGGTTATGTTTATGCTACCTATAACATCTGGGGCACAGGCACCC
TGGTCACCATCTCTTCA

Preferably, the antibody or antigen-binding fragment thereof comprises a heavy chain variable (VH) region encoded by a nucleic acid sequence as substantially set out in SEQ ID No: 143, or a variant or fragment thereof.

The antibody or antigen-binding fragment thereof may comprise a CDR-L1 domain of SEQ ID No: 144, which is provided herein, as follows:

[SEQ ID No: 144]
EDIGYG

Thus, preferably, the antibody or antigen-binding fragment thereof comprises a CDR-L1 domain comprising or consisting of a sequence as substantially set out in SEQ ID No: 144, or a variant or fragment thereof.

The antibody or antigen-binding fragment thereof may comprise a CDR-L2 domain of SEQ ID No: 145, which is provided herein, as follows:

[SEQ ID No: 145]
GAN

Thus, preferably, the antibody or antigen-binding fragment thereof comprises a CDR-L2 domain comprising or consisting of a sequence as substantially set out in SEQ ID No: 145, or a variant or fragment thereof.

The antibody or antigen-binding fragment thereof may comprise a CDR-L3 domain of SEQ ID No: 146, which is provided herein, as follows:

[SEQ ID No: 146]
QQGYSTPPT

Thus, preferably, the antibody or antigen-binding fragment thereof comprises a CDR-L3 domain comprising or consisting of a sequence as substantially set out in SEQ ID No: 146, or a variant or fragment thereof.

Preferably, the antibody or antigen-binding fragment thereof comprises a CDR-L1 domain comprising or consisting of SEQ ID No: 144, a CDR-L2 domain comprising or consisting of SEQ ID No: 145, and/or a CDR-L3 domain comprising or consisting of SEQ ID No: 146. However, preferably the antibody or antigen-binding fragment thereof comprises a CDR-L1 domain comprising or consisting of SEQ ID No: 144, a CDR-L2 domain comprising or consisting of SEQ ID No: 145, and a CDR-L3 domain comprising or consisting of SEQ ID No: 146.

The antibody or antigen-binding fragment thereof may comprise a FR-L1 domain of SEQ ID No: 147, which is provided herein, as follows:

[SEQ ID No: 147]
AYDMTQTPSSLAASVGDTVTITCKAS

Thus, preferably, the antibody or antigen-binding fragment thereof comprises a FR-L1 domain comprising or consisting of a sequence as substantially set out in SEQ ID No: 147, or a variant or fragment thereof.

The antibody or antigen-binding fragment thereof may comprise a FR-L2 domain of SEQ ID No: 148, which is provided herein, as follows:

[SEQ ID No: 148]
LNWYQQKLGIAPKLLIY

Thus, preferably, the antibody or antigen-binding fragment thereof comprises a FR-L2 domain comprising or consisting of a sequence as substantially set out in SEQ ID No: 148, or a variant or fragment thereof.

The antibody or antigen-binding fragment thereof may comprise a FR-L3 domain of SEQ ID No: 149, which is provided herein, as follows:

[SEQ ID No: 149]
TLESGVPSRFSGSGSETDYTLTISSVQAEDAGIYYC

Thus, preferably, the antibody or antigen-binding fragment thereof comprises a FR-L3 domain comprising or consisting of a sequence as substantially set out in SEQ ID No: 149, or a variant or fragment thereof.

The antibody or antigen-binding fragment thereof may comprise a FR-L4 domain of SEQ ID No: 150, which is provided herein, as follows:

[SEQ ID No: 150]
FGAGTMVEIK

Thus, preferably, the antibody or antigen-binding fragment thereof comprises a FR-L4 domain comprising or consisting of a sequence as substantially set out in SEQ ID No: 150, or a variant or fragment thereof.

Preferably, the antibody or antigen-binding fragment thereof comprises a FR-L1 domain comprising or consisting of SEQ ID No: 147, a FR-L2 domain comprising or consisting of SEQ ID No: 148, a FR-L3 domain comprising or consisting of SEQ ID No: 149, and/or a FR-L4 domain comprising or consisting of SEQ ID No: 150. Preferably, however, the antibody or antigen-binding fragment thereof comprises a FR-L1 domain comprising or consisting of SEQ ID No: 147, a FR-L2 domain comprising or consisting of SEQ ID No: 148, a FR-L3 domain comprising or consisting of SEQ ID No: 149, and a FR-L4 domain comprising or consisting of SEQ ID No: 150.

The antibody or antigen-binding fragment thereof may comprise a light chain variable (VL) sequence as set out in SEQ ID No: 151, which is provided herein, as follows:

[SEQ ID No: 151]
AYDMTQTPSSLAASVGDTVTITCKASEDIGYGLNWYQQKLGIAPKLLIYG
ANTLESGVPSRFSGSGSETDYTLTISSVQAEDAGIYYCQQGYSTPPTFGA
GTMVEIK

Preferably, the antibody or antigen-binding fragment thereof comprises a light chain variable region comprising or consisting of a sequence as substantially set out in SEQ ID No: 151, or a variant or fragment thereof.

One embodiment of the nucleotide sequence encoding the light chain variable (VL) region is referred to herein as SEQ ID No: 152, as follows:

[SEQ ID No: 152]
GCATATGATATGACCCAGACTCCATCCTCCCTGGCTGCATCTGTGGGAGA
CACAGTCACCATCACTTGTAAGGCCAGTGAGGACATTGGTTATGGGTTGA
ACTGGTATCAGCAGAAACTAGGGATAGCTCCTAAGCTCCTCATCTATGGG
GCAAACACTTTAGAATCCGGGGTCCCATCGAGGTTCAGTGGCAGCGGATC
AGAGACCGATTACACCCTCACCATCAGCAGCGTGCAGGCTGAAGATGCAG
GAATTTATTACTGTCAGCAAGGATATAGTACCCCTCCTACTTTCGGTGCG
GGGACCATGGTGGAGATCAAA

Preferably, the antibody or antigen-binding fragment thereof comprises a light chain variable (VL) region encoded by a nucleic acid sequence as substantially set out in SEQ ID No: 152, or a variant or fragment thereof.

Preferably, the antibody or antigen-binding fragment thereof comprises at least one, at least two, at least three, at least four, at least five, or at least six CDRs. Preferably, the antibody or antigen-binding fragment thereof comprises at least CDR-H3.

Preferably, the antibody or antigen-binding fragment thereof comprises a CDR-H1 domain comprising or consisting of SEQ ID No: 135, a CDR-H2 domain comprising or consisting of SEQ ID No: 136; a CDR-H3 domain comprising or consisting of SEQ ID No: 137, a CDR-L1 domain comprising or consisting of SEQ ID No: 144, a CDR-L2 domain comprising or consisting of SEQ ID No: 145, and a CDR-L3 domain comprising or consisting of SEQ ID No: 146.

Preferably, the antibody or antigen-binding fragment thereof comprises a heavy chain variable region comprising or consisting of SEQ ID No: 142, and a light chain variable region comprising or consisting of SEQ ID No: 151.

Preferably, the antibody or antigen-binding fragment thereof comprises a heavy chain variable region encoded by a nucleic acid sequence comprising or consisting of SEQ ID No: 143, and a light chain variable region encoded by a nucleic acid sequence comprising or consisting of SEQ ID No: 152.

As shown in FIGS. 7A-7E, the inventors have surprisingly demonstrated that the antibodies and antigen-binding fragments thereof, referred to herein as 1-A2, 4-H3, 1-D5, 3-H9 and 1-G5, comprising a disabled Fc fragment, are able to bind C1-C6 domains of VWF. The Fc fragment is involved in platelet aggregation, and therefore, is associated with an increased risk of blood clotting and thrombosis. As such, it is particularly advantageous to disable the Fc fragment of the antibody and reduce the risk of blood clotting, when treating a patient with a condition caused by platelet-mediated aggregation.

Thus, in one embodiment, the antibody or antigen-binding fragment thereof of the invention comprises a disabled Fc fragment. Preferably, the disabled Fc fragment comprises one or more amino acid substitution that silences or reduces the effector function of the antibody or antigen-binding fragment thereof. Preferably, the disabled Fc fragment comprises one or more amino acid substitution selected from the group consisting of: L234A, L235A, and P329G. Preferably, the disabled Fc fragment comprises the amino acid substitutions L234A, L235A, and P329G.

Thus, most preferably, the antibody or antigen-binding fragment thereof, referred to herein as 1-A2, 4-H3, 1-D5, 3-H9 or 1-G5, comprises a disabled Fc fragment comprising the amino acid substitutions L234A, L235A, and P329G.

Additionally, also as shown in FIGS. 7A-7E, the inventors have demonstrated that the Fab fragments of antibody clones 1-A2, 4-H3, 1-D5, 3-H9 and 1-G5, are surprisingly able to bind C1-C6 domains of VWF. Thus, in another embodiment, the antigen-binding fragment thereof comprises or consists of a Fab fragment. Preferably, the antigen-binding fragment thereof, referred to herein as 1-A2, 4-H3, 1-D5, 3-H9 and 1-G5, comprises or consists of a Fab fragment.

In another embodiment, the inhibitor can be an interfering nucleic acid molecule including: antisense oligonucleotide, siRNA, or dsRNA, which specifically targets a portion of an mRNA encoding one or more of the C1, C2, C3, C4, C5, and/or C6 domain of VWF. A functional interfering nucleic acid molecule, including antisense oligonucleotides, siRNA molecules, or dsRNA molecules, is capable of specifically downregulating a target gene, preferably one or more exon thereof.

In another embodiment, the inhibitor may be a biological agent, a small molecule drug, a protein, a nucleic acid, or a pharmaceutical agent.

Thus, advantageously, the anti-C1-C6-VWF activity of the inhibitor according to the first aspect of the invention means that it has significant utility as a therapeutic agent in its own right, and may be used in the treatment, amelioration or prevention of a condition caused by platelet-mediated aggregation, such as a thrombotic-related condition.

Accordingly, in a second aspect of the invention, there is provided an inhibitor according to the first aspect, for use in therapy.

In a third aspect of the invention, there is provided an inhibitor according to the first aspect, for use in treating, preventing or ameliorating a condition caused by platelet-mediated aggregation.

According to a fourth aspect of the invention, there is provided a method of treating, preventing or ameliorating a condition caused by platelet-mediated aggregation in a subject, the method comprising administering, or having administered, to a patient in need of such treatment, a therapeutically effective amount of an inhibitor according to the first aspect.

The condition caused by platelet-mediated aggregation may be selected from the group consisting of: a thrombotic-related condition; thrombotic thrombocytopenia purpura (TTP) (also referred to as acquired thrombotic thrombocytopenia purpura (aTTP)), acute coronary syndrome (ACS), atherosclerosis, ischemic stroke, atrial fibrillation (AF), acute myocardial infarction (AMI), cardiovascular disease (CVD), thrombosis, unstable angina, stable angina, angina pectoris, embolus formation, deep vein thrombosis, haemolytic uremic syndrome, haemolytic anaemia, acute renal failure, thrombolytic complications, disseminated intravascular coagulation, coronary heart disease, thromboembolic complications, restenosis, chronic unstable angina, peripheral vascular disease, arterial thrombosis, pre-eclampsia, embolism, restenosis, sepsis, vascular inflammation, glomerulonephritis, and thrombotic condition resulting from a coronavirus infection.

Preferably, the use or method in treating, preventing or ameliorating a condition caused by platelet-mediated aggregation comprises inhibiting platelet binding under conditions of high shear rate, i.e. pathological conditions.

The gradient in the blood flow speed (slope of the velocity profile) in the laminar layers is highest at the vessel wall. This shear rate is termed wall shear rate. Under normal physiological flow conditions, the wall shear rate increases from about 10 s-1 in veins to about 15000 s-1 in the smallest arteries, whereas maximal wall shear rates up to 40,000 s″1 have been described for severe atherosclerotic arteries. One possible method for measuring shear rate in vivo, is further discussed in Brands et al., 1999, “An integrated system for the non-invasive assessment of vessel wall and hemodynamic properties of large arteries by means of ultrasound”. Specifically, a system referred to as arterial laboratory (ART-lab), measures radio frequency ultrasound signals to determine haemodynamic properties of arteries. The radio frequency signals received from an echo scanner are acquired by means of a data acquisition system and are then stored on a hard-disk. The assessment of blood flow velocity is based on the estimation of the temporal and spatial mean frequency in a given estimation window, which includes radiofrequency-samples filtered by a clutter filter, to discriminate between reflection and scattering. The temporal and spatial mean frequencies are directly related to velocity by means of the Doppler equation. From this velocity profile, the wall shear rate is calculated based on the maximum value of the derivative of the observed spatial velocity distribution with respect to the radius.

Preferably, the inhibitor is an antibody or antigen-binding fragment thereof. Most preferably, the inhibitor is one of the antibodies from FIG. 8, or an antigen-binding fragment thereof.

It will be appreciated that inhibitors according to the invention (referred to herein as “agents”) may be used in a monotherapy (e.g. the use of an inhibitor alone, more preferably one of the antibodies described herein), for treating, ameliorating or preventing a condition caused by platelet-mediated aggregation. Alternatively, agents according to the invention may be used as an adjunct to, or in combination with, known therapies for treating, ameliorating, or preventing a condition caused by platelet-mediated aggregation, such as aspirin, clopidogrel, abciximab, heparin, warfarin, and direct oral anticoagulants (DOACs) including, dabigatran (Pradaxa), rivaroxaban (Xarelto), apixaban (Eliquis), edoxaban (Savaysa), and betrixaban (Bevyxxa).

The agents according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising agents of the invention may be used in a number of ways. For instance, oral administration may be required, in which case the agents may be contained within a composition that may, for example, be ingested orally in the form of a tablet, capsule or liquid. Compositions comprising agents and medicaments of the invention may be administered by inhalation (e.g. intranasally). Compositions may also be formulated for topical use. For instance, creams or ointments may be applied to the skin.

Agents and medicaments according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with agents used according to the invention is required and which would normally require frequent administration (e.g. at least daily injection).

In a preferred embodiment, agents and medicaments according to the invention may be administered to a subject by injection into the blood stream or directly into a site requiring treatment. Injections may be intravenous (bolus or infusion) or subcutaneous (bolus or infusion), or intradermal (bolus or infusion).

It will be appreciated that the amount of the inhibitor (i.e. agent) that is required is determined by its biological activity and bioavailability, which in turn depends on the mode of administration, the physiochemical properties of the agent, and whether it is being used as a monotherapy or in a combined therapy. The frequency of administration will also be influenced by the half-life of the agent within the subject being treated. Optimal dosages to be administered may be determined by those skilled in the art, and will vary with the particular agent in use, the strength of the pharmaceutical composition, the mode of administration, and the advancement of the thrombotic-related condition. Additional factors depending on the particular subject being treated will result in a need to adjust dosages, including subject age, weight, gender, diet, and time of administration.

Generally, a daily dose of between 0.01 μg/kg of body weight and 100 mg/kg of body weight of agent according to the invention may be used for treating, ameliorating, or preventing a thrombotic-related condition, depending upon which agent. More preferably, the daily dose of agent is between 1 μg/kg of body weight and 100 mg/kg of body weight, more preferably between 10 μg/kg and 10 mg/kg body weight, and most preferably between approximately 100 μg/kg and 10 mg/kg body weight.

The agent may be administered before, during or after onset of a thrombotic-related condition. Daily doses may be given as a single administration (e.g. a single daily injection). Alternatively, the agent may require administration twice or more times during a day. As an example, agents may be administered as two (or more depending upon the severity of the thrombotic-related condition being treated) daily doses of between 0.07 μg and 700 mg (i.e. assuming a body weight of 70 kg). A patient receiving treatment may take a first dose upon waking and then a second dose in the evening (if on a two dose regime) or at 3—or 4-hourly intervals thereafter. Alternatively, a slow release device may be used to provide optimal doses of agents according to the invention to a patient without the need to administer repeated doses. Known procedures, such as those conventionally employed by the pharmaceutical industry (e.g. in vivo experimentation, clinical trials, etc.), may be used to form specific formulations of the agents according to the invention and precise therapeutic regimes (such as daily doses of the agents and the frequency of administration).

In a fifth aspect of the invention, there is provided a pharmaceutical composition comprising an inhibitor according to the first aspect, and optionally a pharmaceutically acceptable vehicle.

The pharmaceutical composition is preferably anti-thrombotic, i.e. a pharmaceutical formulation used in the therapeutic amelioration, prevention or treatment of a condition caused by platelet-mediated aggregation.

The invention also provides in a sixth aspect, a process for making the pharmaceutical composition according to the fifth aspect, the process comprising combining a therapeutically effective amount of an inhibitor as defined in the first aspect, with a pharmaceutically acceptable vehicle.

The inhibitor may be as defined with respect to the first aspect. Preferably, the inhibitor is an antibody or antigen-binding fragment thereof. Most preferably, the inhibitor is one of the antibodies from FIG. 8, or an antigen-binding fragment thereof.

A “subject” may be a vertebrate, mammal, or domestic animal. Hence, medicaments according to the invention may be used to treat any mammal, for example livestock (e.g. a horse), pets, or may be used in other veterinary applications. Most preferably, the subject is a human being.

A “therapeutically effective amount” of the inhibitor is any amount which, when administered to a subject, is the amount of agent that is needed to treat the thrombotic-related condition, or produce the desired effect.

For example, the therapeutically effective amount of inhibitor used may be from about 0.1 ng/kg to about 100 mg/kg, and preferably from about 1 ng/kg to about 10 mg/kg. It is preferred that the amount of inhibitor is an amount from about 10 ng/kg to about 10 mg/kg, and most preferably from about 50 ng/kg to about 5 mg/kg.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The active agent according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, 20) sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The agent may be prepared as a sterile solid composition that may be dissolved or suspended at the time of administration using sterile water, saline, or other appropriate sterile injectable medium.

The agents and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The agents used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

The invention also extends to methods for producing the antibody, or antigen-binding fragment of the first aspect, and antibody, or antigen-binding fragment so produced.

In a seventh aspect, there is provided an antibody or antigen-binding fragment thereof obtained by a method comprising:—
(i) immunising a host organism with one or more of a C1, C2, C3, C4, C5, and/or C6 domain of Von Willebrand Factor (VWF); and
(ii) collecting an antibody or antigen-binding fragment thereof from the host.

The host may be a mammal, and may be a human, rabbit, mouse, or chicken. Preferably, the host is a rabbit. More preferably, the host is a New Zealand White (NZW) rabbit.

The method may comprise immunising the host with human C1-C6-Fc fusion protein. Preferably, the method comprises immunising the host with the C5 domain of VWF.

The method may comprise immunising the host with at least 50 μg, 75 μg, or 100 μg of the immunogen. Preferably, the method comprises subsequently immunising the host with a first boost of at least 25 μg, 35 μg or 50 μg of immunogen. Preferably, the first boost is administered at least 20, 30 or 40 days after the first immunisation. Even more preferably, the method comprises subsequently immunising the host with a second boost of at least 25 μg, 35 μg or 50 μg of immunogen.

Preferably, the method comprises bleeding the host animal, and then preferably collecting the antibody or antigen-binding fragment thereof from the blood, most preferably blood serum. Preferably, the method comprises bleeding the host animal at least 30, 40 or 50 days after the first immunisation.

Preferably, the blood serum is passed through a gravity column with covalently bound peptide-support. Following washing, the antibody or antigen-binding fragment thereof is preferably eluted in buffer, which is preferably acidic buffer, and the solution may then be neutralized. The method may further comprise dialysis against a suitable buffer (e.g. PBS) and, optionally, lyophilisation.

In an eighth aspect, there is provided an antibody or antigen-binding fragment thereof, obtained by a method comprising selecting an antibody or antigen-binding fragment thereof that specifically binds to one or more of a C1, C2, C3, C4, C5, and/or C6 domain of Von Willebrand Factor (VWF) using a phage library.

Preferably, the antibody or antigen-binding fragment thereof, is obtained by a method comprising selecting an antibody or antigen-binding fragment thereof that specifically binds to the C5 domain of VWF using a phage library. Phage display is known to the skilled person, and is described in detail in the examples.

In an ninth aspect of the invention, there is provided a polynucleotide sequence encoding the antibody, or antigen-binding fragment as defined in the first aspect.

In a tenth aspect of the invention, there is provided an expression cassette comprising a polynucleotide sequence according to the ninth aspect.

The polynucleotide sequence encoding the antibody, or antigen-binding fragment of the invention is preferably harboured in a recombinant vector, for example a recombinant vector for delivery into a host cell of interest to enable production of the antibody, or antigen-binding fragment thereof.

Accordingly, in a eleventh aspect of the invention, there is provided a recombinant vector comprising the expression cassette according to the tenth aspect.

The vector encoding the antibody, or antigen-binding fragment may for example be a plasmid, cosmid or phage and/or be a viral vector. Such recombinant vectors are highly useful in the delivery systems of the invention for transforming cells with the nucleotide sequences. The nucleotide sequences may preferably be a DNA sequence, and it is this DNA sequence which encodes the antibody, or antigen-binding fragment.

Recombinant vectors encoding the antibody, or antigen-binding fragment may also include other functional elements. For example, they may further comprise a variety of other functional elements including a suitable promoter for initiating transgene expression upon introduction of the vector in a host cell. For instance, the vector is preferably capable of autonomously replicating in the nucleus of the host cell. In this case, elements which induce or regulate DNA replication may be required in the recombinant vector. Alternatively, the recombinant vector may be designed such that it integrates into the genome of a host cell. In this case, DNA sequences which favour targeted integration (e.g. by homologous recombination) are envisaged. Suitable promoters may include the SV40 promoter, CMV, EF1a, PGK, viral long terminal repeats, as well as inducible promoters, such as the Tetracycline inducible system, as examples. The cassette or vector may also comprise a terminator, such as the Beta globin, SV40 polyadenylation sequences or synthetic polyadenylation sequences. The recombinant vector may also comprise a promoter or regulator or enhancer to control expression of the nucleic acid as required.

The vector may also comprise DNA coding for a gene that may be used as a selectable marker in the cloning process, i.e. to enable selection of cells that have been transfected or transformed, and to enable the selection of cells harbouring vectors incorporating heterologous DNA. For example, ampicillin, neomycin, puromycin or chloramphenicol resistance is envisaged. Alternatively, the selectable marker gene may be in a different vector to be used simultaneously with the vector containing the transgene. The cassette or vector may also comprise DNA involved with regulating expression of the nucleotide sequence, or for targeting the expressed polypeptide to a certain part of the host cell.

Purified vector may be inserted directly into a host cell by suitable means, e.g. direct endocytotic uptake. The vector may be introduced directly into a host cell (e.g. a eukaryotic or prokaryotic cell) by transfection, infection, electroporation, microinjection, cell fusion, protoplast fusion, calcium phosphate, cationic lipid-based lipofection, polymer or dendrimer-based methods or ballistic bombardment.

Alternatively, vectors of the invention may be introduced directly into a host cell using a particle gun.

Alternatively, the delivery system may provide the polynucleotide to the host cell without it being incorporated in a vector. For instance, the nucleic acid molecule may be incorporated within a liposome or virus particle. Alternatively a “naked” polynucleotide may be inserted into a host cell by a suitable means e.g. direct endocytotic uptake.

In an twelfth aspect of the invention, there is provided a host cell comprising the polynucleotide sequence according to the ninth aspect, the expression cassette according to the tenth aspect, or the vector according to the eleventh aspect.

The host cell may be a eukaryotic or prokaryotic host cell. Preferably, the host cell is a eukaryotic host cell. More preferably, the host cell is a mammalian host cell such as NSo murine myeloma cells, PER.C6® human cells, Human embryonic kidney 293 cells or Chinese hamster ovary (CHO) cells. Most preferably, the host cell is a CHO cell.

In a thirteenth aspect, there is provided a method of preparing the antibody, or antigen-binding fragment according to the first aspect, the method comprising:
a) introducing, into a host cell, the vector of the eleventh aspect; and
b) culturing the host cell under conditions to result in the production of the antibody, or antigen-binding fragment according to the first aspect.

The host cell of step a) may be a eukaryotic or prokaryotic host cell. Preferably, the host cell is a eukaryotic host cell. More preferably, the host cell is a mammalian host cell such as NSo murine myeloma cells, PER.C6® human cells, Human embryonic kidney 293 cells or Chinese hamster ovary (CHO) cells. Most preferably, the host cell is a CHO cell.

The method may further comprise (c) harvesting, centrifuging and/or filtering the cell culture media to obtain a cell culture supernatant comprising the antibody or antigen binding fragment thereof.

The method may further comprise (d) separating and purifying the antibody or antigen binding fragment thereof from the cell culture supernatant. Preferably, purification is performed by at least one chromatographic step.

Suitable chromatographic steps include affinity chromatography and/or ion exchange chromatography. Preferably, affinity chromatography is protein A chromatography. Ion exchange chromatography may be anionic exchange chromatography and/or cationic exchange chromatography.

Preferably, step (d) comprises separating and purifying the antibody or antigen binding fragment thereof from the cell culture supernatant by:
i) protein A chromatography;
ii) anionic exchange chromatography; and/or
iii) cationic exchange chromatography.

The method may further comprise (e) filtering the purified antibody or antigen binding fragment thereof resulting from step (d). Preferably, step (e) comprises virus filtration. Thus, preferably the purified antibody or antigen binding fragment thereof resulting from step (d) is filtered using a virus filtration membrane. Suitable membranes would be known to those skilled in the art.

As discussed herein, VWF expression is increased in a number of conditions caused by platelet-mediated aggregation, including ischemic stroke, heart attack, acquired thrombotic thrombocytopeniarpura and atrial fibrillation. Thus, given that the antibodies of the invention are able to bind to one or more of the C1, C2, C3, C4, C5, and/or C6 domain of VWF, the antibodies or antigen-binding fragments thereof may be used as a robust diagnostic tool by detecting the presence, and determining the concentration of, VWF.

Thus, in a fourteenth aspect, there is provided the antibody or antibody binding fragment thereof according to the first aspect, for use in diagnosis or prognosis.

According to a fifteenth aspect of the invention, there is provided the antibody or antibody binding fragment thereof according to the first aspect, for use in diagnosing or prognosing a condition caused by platelet-mediated aggregation.

According to the sixteenth aspect, there is provided a method of diagnosing or prognosing a condition caused by platelet-mediated aggregation in a subject, the method comprising detecting VWF in a biological sample obtained from the subject with the antibody or antibody binding fragment thereof according to the first aspect.

The condition caused by platelet-mediated aggregation may be selected from the group consisting of: a thrombotic-related condition; thrombotic thrombocytopenia purpura (TTP) (also referred to as acquired thrombotic thrombocytopenia purpura (aTTP)), acute coronary syndrome (ACS), atherosclerosis, ischemic stroke, atrial fibrillation (AF), acute myocardial infarction (AMI), cardiovascular disease (CVD), thrombosis, unstable angina, stable angina, angina pectoris, embolus formation, deep vein thrombosis, haemolytic uremic syndrome, haemolytic anaemia, acute renal failure, thrombolytic complications, disseminated intravascular coagulation, coronary heart disease, thromboembolic complications, restenosis, chronic unstable angina, peripheral vascular disease, arterial thrombosis, pre-eclampsia, embolism, restenosis, sepsis, vascular inflammation, glomerulonephritis, and thrombotic condition resulting from a coronavirus infection (e.g. COVID-19).

The method may be an in vitro or ex vivo method. Preferably, the method is an in vitro method.

The use or method may comprise determining the level of expression of VWF in a subject, preferably wherein an increase in the concentration of VWF in the biological sample when compared to a reference concentration from a healthy control population is indicative of a condition caused by platelet-mediated aggregation or a poor prognosis.

In one embodiment, a 1 fold increase of VWF when compared to the reference from a healthy control population is indicative of a condition caused by platelet-mediated aggregation or a poor prognosis. In one embodiment, a 2 fold, 3 fold, 4 fold or 5 fold increase of VWF when compared to the reference from a healthy control population is indicative of a condition caused by platelet-mediated aggregation or a poor prognosis. In one embodiment, a 10 fold, 50 fold or 100 fold increase of VWF when compared to the reference from a healthy control population is indicative of a condition caused by platelet-mediated aggregation or a poor prognosis.

According to the seventeenth aspect of the invention, there is provided a kit for diagnosing a subject suffering from a condition caused by platelet-mediated aggregation, or for providing a prognosis of the subject's condition, the kit comprising an antibody or antigen-binding fragment thereof according to the first aspect for detecting VWF in a sample from a test subject.

The kit may further comprise instructions for use and/or a receptacle for obtaining a biological sample from a subject.

Preferably, the condition caused by platelet-mediated aggregation is as defined above.

Prognosis may relate to determining the therapeutic outcome in a subject that has been diagnosed with a condition caused by platelet-mediated aggregation. Prognosis may relate to predicting the rate of progression or improvement and/or the duration of a condition caused by platelet-mediated aggregation in a subject, the probability of survival, and/or the efficacy of various treatment regimes. Thus, a poor prognosis may be indicative of progression of a condition caused by platelet-mediated aggregation, low probability of survival and reduced efficacy of a treatment regime. A favourable prognosis may be indicative of resolution of a condition caused by platelet-mediated aggregation, high probability of survival and increased efficacy of a treatment regime.

Preferably, the sample comprises a biological sample. The sample may be any material that is obtainable from a subject from which protein is obtainable.

The biological sample may be tissue or a biological fluid. The biological sample may be any material that is obtainable from the subject from which blood plasma, endothelial cells, megakaryocytes, and platelets are obtainable. Furthermore, the sample may be blood, plasma, serum, spinal fluid, urine, sweat, saliva, tears, breast aspirate, breast milk, prostate fluid, seminal fluid, vaginal fluid, stool, cervical scraping, cytes, amniotic fluid, intraocular fluid, mucous, moisture in breath, animal tissue, cell lysates, tumour tissue, hair, skin, buccal scrapings, lymph, interstitial fluid, nails, bone marrow, cartilage, prions, bone powder, ear wax, lymph, granuloma, cancer biopsy or combinations thereof.

The sample may be a liquid aspirate. For example, the sample may be bronchial alveolar lavage (BAL), ascites, pleural lavage, or pericardial lavage.

The sample may comprise blood, urine, tissue etc. In one preferred embodiment, the biological sample comprises a blood sample. The blood may be venous or arterial blood. Blood samples may be assayed immediately. Alternatively, the blood sample may be stored at low temperatures, for example in a fridge or even frozen before the method is conducted. Alternatively, the blood sample may be stored at room temperature, for example between 18 to 22 degrees Celsius, before the method is conducted. The blood sample may comprise comprises blood serum. The blood sample may comprise blood plasma. Preferably, however the detection is carried out on whole blood and most preferably the blood sample is peripheral blood.

The blood may be further processed before the use of the first aspect is performed. For instance, an anticoagulant, such as citrate (such as sodium citrate), hirudin, heparin, PPACK, or sodium fluoride may be added. Thus, the sample collection container may contain an anticoagulant in order to prevent the blood sample from clotting.

Preferably, the sample may comprise blood plasma, endothelial cells, megakaryocytes, and/or platelets.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-194 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example,

ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, in those of SEQ ID Nos: 1 to 194 that are amino acid sequences.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

FIG. 1 provides a schematic of VWF peptide structure showing the propeptide and mature VWF regions. Platelet binding is predominantly via the A1 and A3 domains, which are targeted by current therapies, including Caplacizumab. The inhibitors of the invention target one or more of the C1-C6 domains.

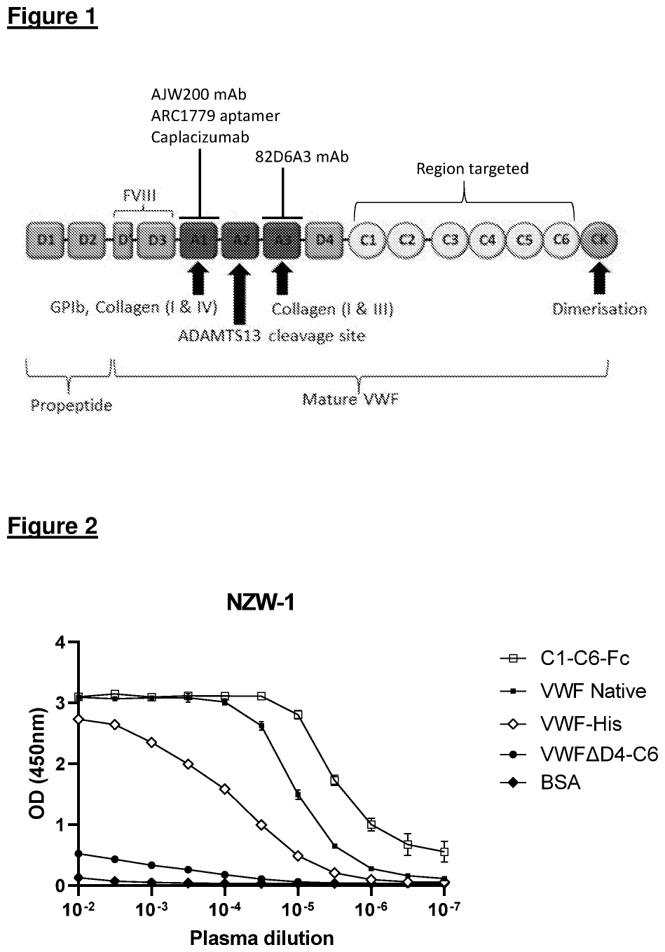

FIG. 2 shows an ELISA-based reactivity screening of plasma from NZW-1 rabbits immunised with human C1-C6-Fc protein, towards recombinant VWF proteins, illustrating that the plasma showed good reactivity towards human native VWF and C1-C6-Fc recombinantly produced protein.

FIGS. 3A-H show the ELISA binding of 8 unique anti-VWF antibodies to native VWF protein and recombinant C1-C6 protein, demonstrating that a number of the antibodies bind specifically to one or more of the C1-C6 domains.

FIG. 4 shows surface plasmon resonance (SPR) affinity determination of 5 selected anti-VWF monoclonal antibodies to human native VWF.

FIG. 5 illustrates the results of a platelet flow assay at a high shear rate (10,000s-1), demonstrating the ability of the antibodies to reduce platelet aggregation under pathological conditions.

FIGS. 6A-C show the results of a whole blood flow assay under normal (1500s-1) and high (5000s-1) shear rates, for 4-H3, 1-A2 and 1-G5 antibodies, demonstrating their ability to inhibit platelet capture under pathological conditions, whilst maintaining the normal haemostatic function of VWF.

FIGS. 7A-E show the ELISA-based reactivity screening of 5 monoclonal antibodies (Fc disabled L234A L235A-P329G null-effector backbone, and Fab fragment antibodies) towards C1-C6-Fc.

FIG. 8 is a table showing the various sequences of eight embodiments of the anti-VWF antibody of the invention. FR=framework region; CDR=complementarity determining region; VH=variable heavy chain sequence; VL=variable light chain sequence; HC=constant heavy chain sequence; LC=constant light chain sequence.

FIG. 9 is a table showing SPR affinity determination of five selected monoclonal antibodies to human VWF individual C-domains. Five rabbit-derived monoclonal antibodies were selected for affinity determination (KD) as well as on-rate (Ka) and off-rate determination (kd) for binding to individual human VWF C-domains. All five monoclonal antibodies showed binding to the C5 domain with sub-nM affinities. Weaker binding was also observed to C4 (3-H9) and C3 (1-D5).

FIG. 10 is a table showing the humanised mAb sequences.

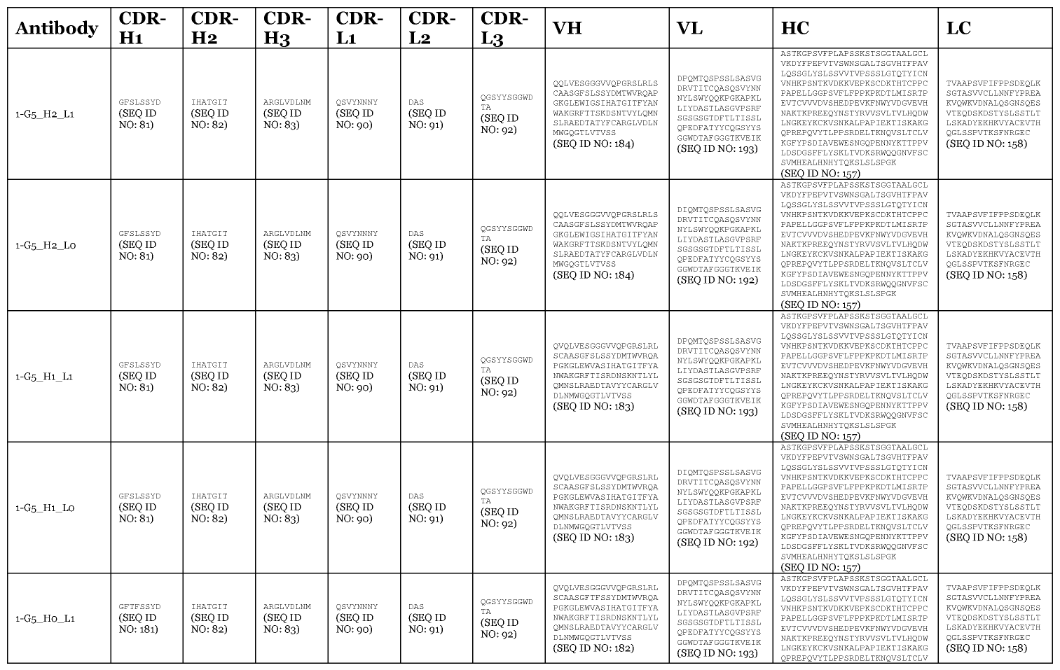

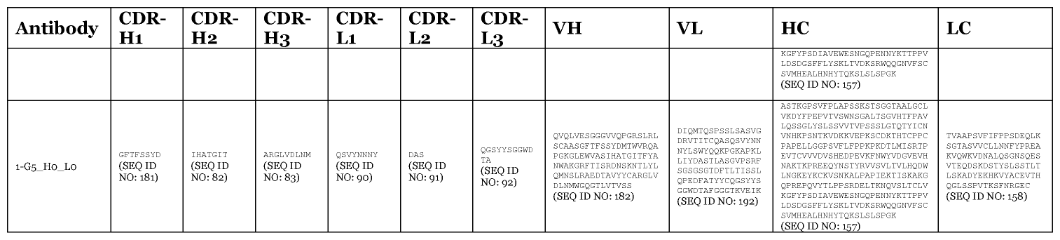

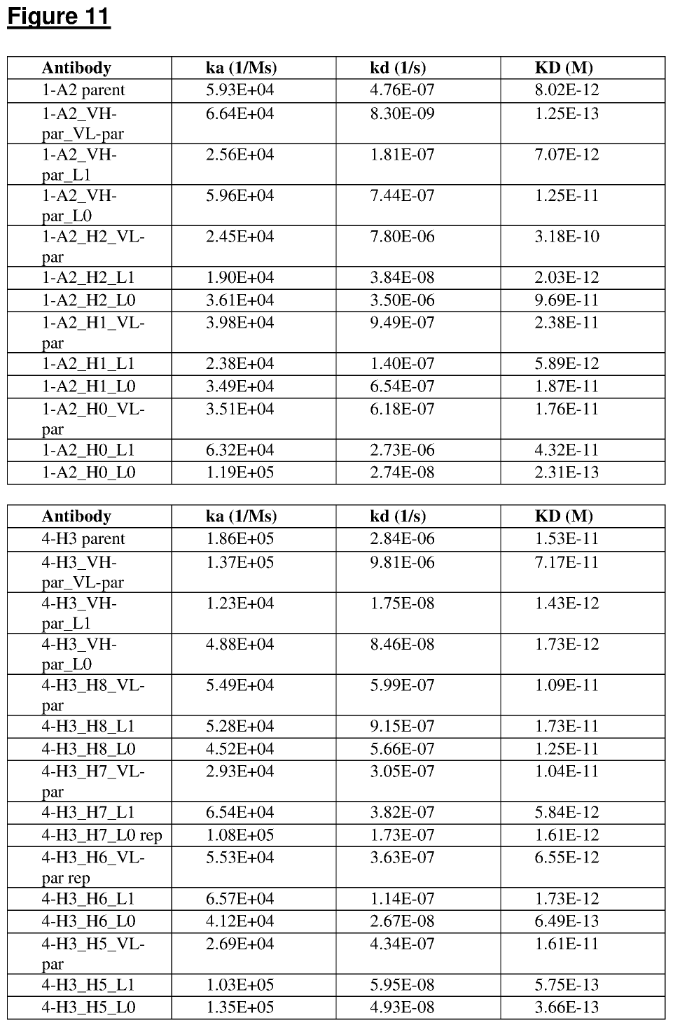

FIG. 11 is a table showing SPR affinity determination to human full-length VWF of humanised monoclonal antibodies. All humanised monoclonal antibodies were selected for affinity determination (KD) as well as on-rate (Ka) and off-rate determination (kd) for binding to human full-length VWF. All clones showed long off-rates and sub nM affinities.

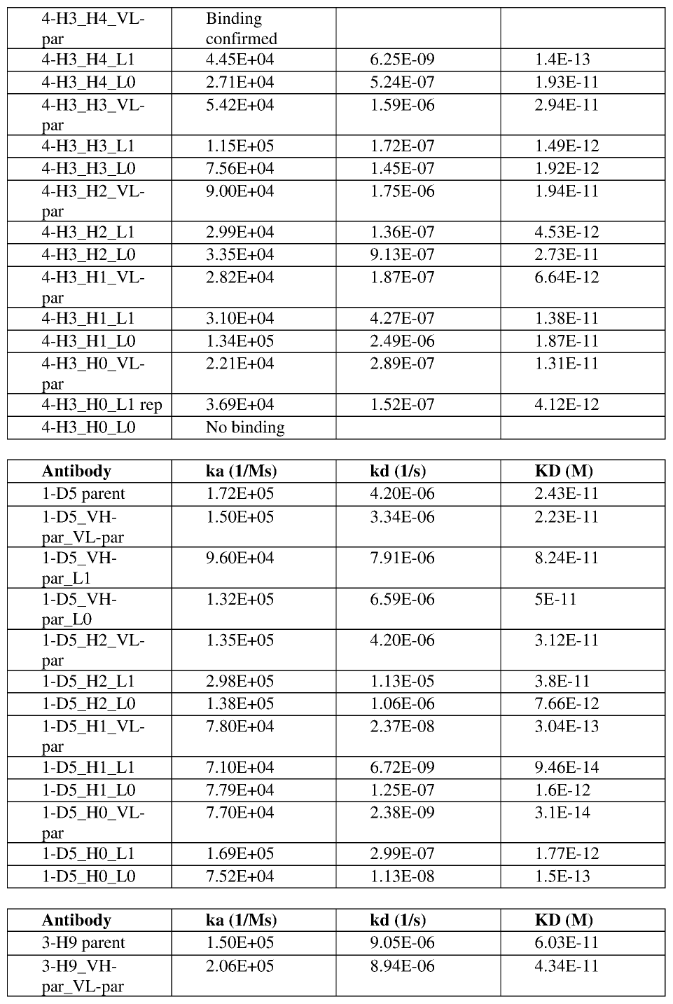

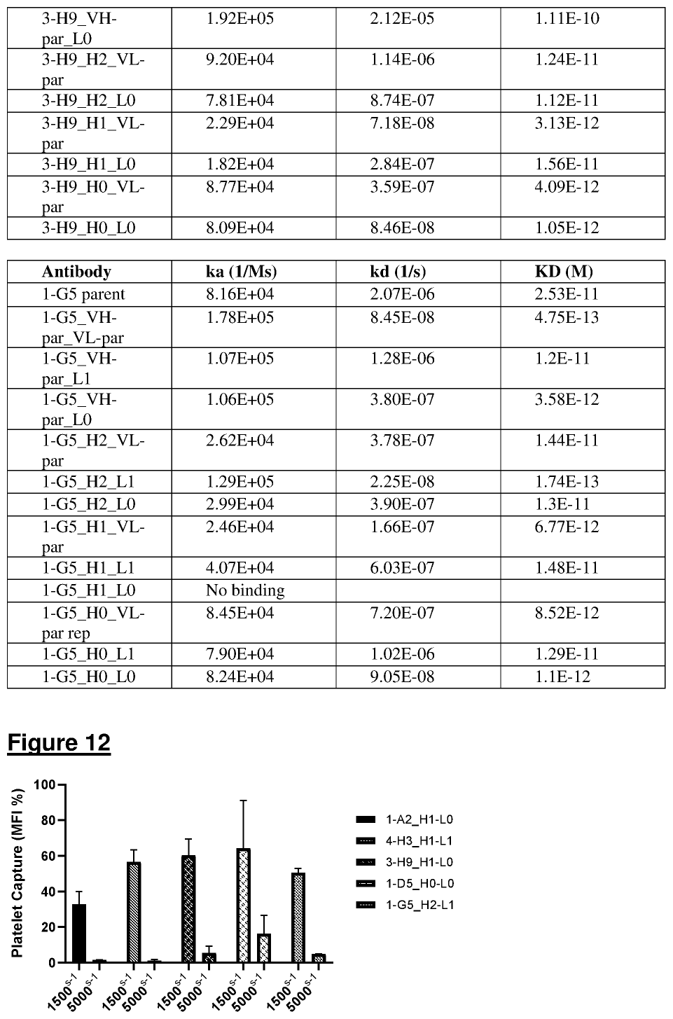

FIG. 12 shows the results of selected humanised anti-VWF antibodies in whole blood flow assay under normal and high shear rates. Humanised antibodies inhibit platelet capture under high shear rate (5000s-1) to a greater extent than under normal shear rate conditions (1500s-1) in a whole blood flow assay, indicating that these antibodies can block the prothrombotic function of VWF while maintaining its normal hemostatic function.

### EXAMPLES

The accumulation of VWF has been associated with an increased risk to a number of conditions caused by platelet-mediated aggregation, including various cardiovascular diseases and thrombotic-related conditions. Currently, however, therapies target the essential A1 domain for VWF, inhibiting platelet binding required for haemostasis, leading to an increased bleeding risk in patients. Therefore, the inventors set out to inhibit a previously untargeted region of VWF, i.e. the C1-C6 domains. The inventors developed antibodies that are capable of specifically binding to the C1-C6 region (i.e. one or more of the C1-C6 domains as shown in FIG. 1), providing an improved treatment for a number of thrombotic-related conditions. As discussed below, several novel C5-targeting antibodies (and also humanised antibodies) have been produced which exhibit the desired effects (i.e. capable of inhibiting platelet capture under high shear rate, indicating that these antibodies are functionally active), and which could therefore be used in therapy and diagnosis.

### Example 1—Generation of Anti-VWF Antibodies

New Zealand White (NZW) rabbits were immunised with 100 μg of recombinantly produced human C1-C6-Fc protein. After 21 days the rabbits received the first boost of 50 μg human C1-C6-Fc and after 42 days, the animals received their second boost of 50 μg C1-C6-Fc. Blood was withdrawn for analysis 52 days after the first immunisation.

20) Target proteins (C1-C6-Fc, VWFAD4-C6 and BSA: 100 ng/well; VWF-His: 500 ng/well and native VWF: 20 ng/well) were immobilized overnight on ELISA plates, blocked and incubated with a semi-log dilution series of NZW-1 rabbit plasma samples (starting dilution 1:100). Antibody binding was detected with anti-rabbit-HRP and TMB.

An illustration of rabbit plasma reactivity towards recombinant VWF proteins is presented in FIG. 2. Plasma from NZW-1 rabbits showed good reactivity towards human native VWF and C1-C6-Fc recombinantly produced protein. Plasma showed, to a lesser extent, reactivity towards recombinantly produced VWF-His tagged protein. There was minimal background reactivity to BSA and a recombinant fragment of VWF with C1-C6 domains deleted (VWFAD4-C6). NZW-1 rabbit was selected for pre-harvest boost and subsequent library generation.

### Example 2—Phage Library Generation and Screening

Following TRIzol-based splenocyte and bone marrow RNA isolation from C1-C6-Fc immunized NZW-1 rabbit, integrity of RNA samples was evidenced by clear presence of

S28 and S18 rRNA bands. RNA was reverse transcribed into cDNA using SuperScript III and cDNA quality was confirmed by PCR amplification of GAPDH and subsequent agarose gel electrophoresis.

PCR amplification of VKs and VHs on splenocyte derived cDNA and on bone marrow derived cDNA. VK and VH fragments were amplified using constant region FR1 forward and FR4 reverse primers containing a Sfil restriction site. The expected size of products was ˜400 bp. For amplification of VK and VH genes, diverse forward and reverse primers were used. PCR products per origin, spleen or bone marrow, were pooled and fragments of the correct size were gel-purified and used for scFv overlap-assembly PCR. Subsequently, gel-purified assembly products of the correct size were Sfil digested and used for generation of two scFv phage libraries, i.e. one library generated from splenocyte derived V-genes and one library generated from bone marrow derived V-genes.

Following large scale ligation of the scFv repertoires in proprietary phagemid vector and subsequent transformation in E. coli TG-1 bacteria, a total number of 8.9×10\hat{ }7 transformants was obtained. 180 randomly picked colonies (90 clones derived from each spleen and bone marrow libraries) were analysed by PCR for presence of full-length scFv insert. 86 out of 90 selected full-length insert containing—clones (45 clones derived from each spleen and bone marrow libraries) were subsequently correctly sequenced (based on quality trace data) of which 60 proved to contain a correct full scFv insert, yielding a final library of 5.9×10\hat{ }7 correct full scFv-containing transformants.

The final phage library underwent 4 rounds of selection against human C1-C6-Fc and human native VWF proteins. The output from the 4 rounds of selection were screened for binding to C1-C6-Fc, VWF-His, VWF-AD4-C6, hIgG, BSA and Streptavidin. A number of reactive clones were selected for sequencing.

Overall, scFv sequences obtained from the combined initial and additional sequencing procedures could be categorized into 5 VH-CDR3 families. A total of 8 unique VH— and 8 unique VL-sequences derived from 8 unique scFv sequences were identified. These sequences were cloned into a human IgG1 expression vector and transiently expressed and purified. Purification of the recombinant antibodies was carried out using Protein A.

### Example 3—ELISA Experiment Showing Binding of Anti-VWF Antibodies to Native VWF protein and recombinant C1-C6 protein

Purified monoclonal antibodies originally derived from scFvs selected from rabbit immune library were tested for binding towards C1-C6-Fc, VWFAD4-C6, BSA (100 ng/well) and human native VWF (Imperial College, 20 ng/well). The 8 unique sequences were cloned into an IgG1 human expression vector and expressed as monoclonal antibodies. Protein targets were immobilized overnight on ELISA plates, blocked and incubated with a semi-log dilution series of purified recombinant monoclonal antibodies (starting concentration 10 μg/ml), averages of duplicate values are shown. Antibody binding towards human native VWF, VWFAD4-C6 and BSA were detected with antihuman IgG-HRP or anti-human IgG-HRP and TMB staining. ELISA reactivities towards C1-C6-Fc were performed in a separate ELISA experiment and were detected with anti-human IgGκ-HRP and TMB staining. Results are shown in FIGS. 3A-3H. Clones 4-H9 (FIG. 3E), 4-C6 (FIG. 3F) and 4-B12 (FIG. 3H) only showed reactivity to C1-C6 domain. Clones 1-G5 (FIG. 3G), 3-H9 (FIG. 3D), 4-H3 (FIG. 3C), 1-D5 (FIG. 3B) and 1-A2 (FIG. 3A) showed positive reactivity to both native VWF and C1-C6 protein. All 20) clones showed minimal binding to VWF without the C1-C6 domain (VWFAC1-C6).

### Example 4—Binding of anti-VWF antibodies to human native VWF as determined by SPR

The surface plasmon resonance (SPR) experiments were performed using a Biacore 8K (Cytiva) equipped with a research grade Protein A series S sensor chip. The antibodies at a concentration of 0.156 μg/ml or 0.312 μg/ml in 10 mM Hepes pH 7.4 containing 300 mM NaCl, 3 mM EDTA and 0.05% P20 buffer, were immobilised onto a protein A series S sensor chip to a density of approximately 12RU on flow cell 2. Flow cell 1 was left blank to serve as a reference surface. To collect single cycle kinetic binding, the surface was initially primed with 10 mM glycine-HCl pH 1.5 for 30 seconds at 50 μl/min followed by running buffer (10 mM Hepes pH7.4 containing 300 mM NaCl, 3 mM EDTA and 0.05% P20) 60 seconds at 30 μl/min, before injecting the native VWF (305 kDa) in 10 mM Hepes pH 7.4 containing 300 mM NaCl, 3 mM EDTA and 0.05% P20 over two flow cells at a concentration of 4.4, 6.6, 9.9, 14.8, 22, 33, and 50 nM, at a flow rate of 30 μl/min, compartment temperature of 10° C. and flow cell temperature of 25° ° C. The complex was allowed to associate and dissociate for 120 seconds and 7200 seconds respectively. The surfaces were regenerated with 30 see injections of 10 mM glycine-HCl pH1.5.

Data were collected at a rate of 10 Hz. The data were fit to a simple 1:1 interaction model using the global data analysis option available within the Biacore Insight Evaluation software version 3.0.12.15655. Results are shown in FIG. 4.

Five clones were selected for affinity determination (KD) as well as on-rate (Ka) and off-rate determination (kd) for binding to native VWF. All 5 clones showed reproducible kinetics, with long off-rates and sub nM affinities.

### Example 5—Platelet Flow Assay

Flow slides were coated with purified VWF and perfused with plasma-free blood at high shear rate (10000-1). Plasma free blood is whole blood with the plasma fraction removed, and only red blood cells and platelets remain. In the absence of soluble VWF, platelets attach to the VWF surface, but no aggregates of platelets and VWF form. When soluble VWF is added, aggregates of platelets and VWF form. The shear force is so high the soluble VWF can unfold in solution and capture platelets (see isotype control image). The addition of the antibodies (10 μg/ml) prevents this occurring to varying degrees.

The images generated from this plasma-free flow assay are presented in FIG. 5, with platelet capture shown in white (white “clumps” are indicative of normal aggregation). The isotype control shows the level of platelet capture in the absence of an anti-VWF

C1-C6 antibody. In the presence of clones 1-A2 and 4-H3, platelet capture is ablated. Clones 1-D5, 3-H9 and 1-G5 also reduce platelet and thrombi formation under pathological conditions. Accordingly, the results show that platelet aggregation is reduced in the presence of 5 different C1-C6 mAbs at a high shear ‘pathological’ rate (10,000s-1), confirming that all 5 antibodies are functionally active. These are the same clones that demonstrated the greatest binding to native VWF as well as C1-C6 (see Example 3).

### Example 6—Whole Blood Flow Assay

Slides were coated with collagen, perfused with whole blood with the test antibody at concentrations as indicated in FIG. 6. Mean Fluorescent Intensity (MFI) was calculated from images taken after 5 minutes of flow and normalised to the isotype control (MFI %). The same blood donor was used for each flow rate.

As illustrated in FIGS. 6A-6C, 4-H3, 1-A2 and 1-G5 antibodies inhibit platelet capture under high shear rate (5000s-1) to a greater extent than under normal shear rate conditions (1500s-1) in a whole blood flow assay. This demonstrates that these antibodies can block the prothrombotic function of VWF while maintaining its normal haemostatic function.

### Example 7—ELISA Experiment Showing the Binding of Fc Disabled Anti-VWF Antibodies, and Anti-VWF Fab Fragments, Towards C1-C6-Fc

An ELISA-based reactivity screening was carried out on: (i) purified monoclonal vWF antibodies comprising an Fc disabled L234A L235A-P329G null-effector backbone, and on (ii) vWF Fab fragments, to test for their binding towards C1-C6-Fc (100 ng/well). Protein targets were immobilized overnight on ELISA plates, blocked and incubated with a semi-log dilution series of purified recombinant monoclonal antibodies or Fab fragments (starting concentration 10 μg/ml, 11 dilutions), averages of duplicate values are shown. Antibody or Fab fragment binding towards C1-C6-Fc was detected with anti-human IgGκ-HRP and TMB staining.

As shown in FIGS. 7A-7E, the Fab fragments and Fc disabled antibodies of clones 1-A2 (FIG. 7A), 4-H3 (FIG. 7B), 1-D5 (FIG. 7C), 3-H9 (FIG. 7D) and 1-G5 (FIG. 7E), all showed positive reactivity and binding to C1-C6-Fc.

### Example 8—SPR Analysis of Five mAbs to Individual C-Domains

Antibodies were captured on Protein A sensor chip. The system was purged using running buffer (10 mM HEPES pH 7.4, 300 mM NaCl, 3 mM EDTA, 0.05% P20) and a series S Protein A chip was docked in the Biacore T200. The surface was conditioned with 10 mM glycine-HCl pH 1.5 regeneration solution (3 injections). Each antibody was diluted to ˜0.8 μg/ml in running buffer to capture ˜350 RU on flow cells 2, 3 and 4 (flow cell 1 used as in-line reference cell). Single-cycle kinetic analysis of each C-domain binding to antibodies was performed using the following parameters:

| | |
|---|---|
| Flow cell | 1-4 |
| Flow rate (μl/min) | 30 |
| Sample compartment | 10 |
| temperature (° C.) | |
| Flow cell temperature (° C.) | 25 |
| Contact time (s) | 120 |
| Dissociation time (s) | 7200 |
| Individual C-domain | 500, 166.67, 55.56, 18.52, 6.17 |
| concentrations (nM) | 100, 33.33, 11.11, 3.70, 1.23 |
| | 20, 6.67, 2.22, 0.74, 0.25 |

The chip surface was regenerated after each cycle with 10 mM glycine-HCl pH 1.5 for seconds at 50 μl/min. Affinity and kinetics are reported in FIG. 9, for each antibody tested against human each C-domain.

As illustrated in FIG. 9, all five of the monoclonal antibodies showed binding to the C5 domain with sub-nM affinities. Weaker binding was also observed to C4 (3-H9) and C3 (1-D5).

### Example 9—Cloning and Expression of the Humanised Variants

The DNA expression constructs encoding the humanised antibody variants were prepared de novo by build-up of overlapping oligonucleotides including restriction sites for cloning into mammalian expression vectors as well as a human signal sequence. HindIII and Spel restriction sites were introduced to frame the VH domain containing the signal sequence for cloning into mammalian expression vectors containing the human γ 1 constant region. HindIII and BsiWI restriction sites were introduced to frame the VL domain containing the signal sequence for cloning into mammalian expression vector containing the human kappa constant region. Expression plasmids encoding the heavy and light chains respectively were transiently co-transfected into HEK 293 6E cells and expressed to produce antibody. Preparations were purified using protein A and concentrations were measured using a Nanodrop (Thermo Scientific).

### Example 10—SPR Analysis of Humanised mAbs to Human Full-Length VWF

Antibodies were captured on Protein A sensor chip. The system was purged using running buffer (10 mM HEPES pH 7.4, 300 mM NaCl, 3 mM EDTA, 0.05% P20) and a series S Protein A chip was docked in the Biacore T200. The surface was conditioned with 10 mM glycine-HCl pH 1.5 regeneration solution (3 injections). Each antibody was diluted to ˜0.8 μg/ml in running buffer to capture ˜350 RU on flow cells 2, 3 and 4 (flow cell 1 used as in-line reference cell). Single-cycle kinetic analysis of human full-length VWF binding to antibodies was performed using the following parameters:

| | |
|---|---|
| Flow cell | 1-4 |
| Flow rate (μl/min) | 30 |
| Sample compartment | 10 |
| temperature (° C.) | |
| Flow cell temperature (° C.) | 25 |
| Contact time (s) | 120 |
| Dissociation time (s) | 7200 |
| Individual C-domain | 500, 166.67, 55.56, 18.52, 6.17 |
| concentrations (nM) | 100, 33.33, 11.11, 3.70, 1.23 |
| | 20, 6.67, 2.22, 0.74, 0.25 |

The chip surface was regenerated after each cycle with 10 mM glycine-HCl pH 1.5 for at 50 μl/min. Affinity and kinetics are reported for each antibody tested against human full-length VWF. As illustrated in FIG. 11, all humanised monoclonal antibody clones showed long-off rates and sub nM affinities for human full-length VWF.

### Example 11—Inhibition of Platelet Capture by Humanised Anti-VWF Antibodies in Whole Blood Flow Assay Under Normal and High Shear Rates

Slides were coated with collagen, perfused with whole blood with test antibody at concentrations as indicated. Mean Fluorescent Intensity (MFI) was calculated from images taken after 5 min of flow and normalised to the isotype control (MFI %). Same blood donor was used for each flow rate.

As illustrated in FIG. 12, all five of the humanised antibodies inhibit platelet capture under high shear rate (5000s-1) to a greater extent than under normal shear rate conditions (1500s-1) in a whole blood flow assay, indicating that these antibodies can block the prothrombotic function of VWF while maintaining its normal hemostatic function. Accordingly, these results confirm that the humanised monoclonal antibodies are functionally active.

### Discussion & Conclusions

The inventors have identified the C1-C6 domains of the VWF protein as being important for VWF function in pro-thrombotic, pathological conditions, and have therefore developed antibodies that are capable of binding to, and inhibiting, C1-C6 VWF function. For example, as shown in FIGS. 3A-3H and FIGS. 7A-7E, the inventors have developed a number of antibodies and antigen-binding fragments thereof that have demonstrated the ability to specifically target the C1-C6 domains of

VWF. In particular, as shown in FIG. 9, the inventors have demonstrated that the monoclonal antibodies of the invention bind to the C5 domain of VWF. Furthermore, as shown in FIGS. 5 and 6, the inventors have demonstrated that by targeting the C1-C6 domains, the antibodies can reduce platelet aggregation under high shear pathological rates, but under normal conditions, platelet capture is maintained.

Additionally, the inventors have generated humanised versions of the antibodies according to the invention, and have demonstrated that the humanised antibodies can bind to human full-length VWF with high affinity. The inventors have also demonstrated that the humanised antibodies are capable of inhibiting platelet capture under high shear rate, indicating that these humanised monoclonal antibodies are functionally active.

The current anti-VWF therapies target and inhibit the A1 domain of VWF, and as such, inhibit platelet binding under low shear conditions. This prevents normal haemostasis taking place, resulting in a severe bleeding risk in patients. The inventors have demonstrated that by targeting the C1-C6 domains of VWF, platelet binding can be inhibited under high shear pathological rates only, not under low shear rates associated with normal haemostasis. Accordingly, the inventors have identified a novel strategy for preventing or treating thrombotic-related conditions, without increasing the risk of severe bleeding.

1. An inhibitor that specifically binds to one or more of a C1, C2, C3, C4, C5, and/or C6 domain of Von Willebrand Factor (VWF).

2. The inhibitor according to claim 1, wherein the inhibitor specifically binds to the C5 domain of VWF.

3. The inhibitor according to either claim 1 or claim 2, wherein the inhibitor does not substantially bind to an A1, A2 and/or A3 domain of VWF.

4. The inhibitor according to any preceding claim, wherein the inhibitor binds to a region between amino acid positions 2255 and 2722 of VWF, as substantially set out in SEQ ID No: 1.

5. The inhibitor according to any preceding claim, wherein the inhibitor binds to one or more amino acids in SEQ ID No: 2, or a variant or fragment thereof.

6. The inhibitor according to any preceding claim, wherein the inhibitor binds to one or more amino acids in SEQ ID No: 3, 4, 5, 6, 7, and/or 8, or a variant or fragment thereof.

7. The inhibitor according to any preceding claim, wherein the inhibitor is a biological agent, a small molecule drug, a protein, a nucleic acid, or a pharmaceutical agent.

8. The inhibitor according to claim 7, wherein the inhibitor is an antisense oligonucleotide, siRNA, or dsRNA, which specifically targets a portion of an mRNA encoding one or more of the C1, C2, C3, C4, C5, and/or C6 domain of VWF.

9. The inhibitor according to any one of claims 1 to 6, wherein the inhibitor is an antibody, or an antigen-binding fragment thereof.

10. The antibody or antigen-binding fragment thereof according to claim 9, wherein the antibody or antigen-binding fragment thereof is a monoclonal antibody or an antigen-binding fragment thereof, optionally wherein the antibody or antigen-binding fragment thereof comprises a disabled Fc fragment, optionally wherein the disabled Fc fragment comprises one or more amino acid substitution selected from the group consisting of: L234A, L235A, and P329G.

11. The antibody or antigen-binding fragment thereof according to either claim 9 or claim 10, wherein the antibody or antigen-binding fragment thereof comprises:
(i) a CDR-H1 domain comprising SEQ ID No: 9, a CDR-H2 domain comprising SEQ ID No: 10, a CDR-H3 domain comprising SEQ ID No: 11, a CDR-L1 domain comprising SEQ ID No: 18, a CDR-L2 domain comprising SEQ ID No: 19 and/or a CDR-L3 domain comprising SEQ ID No: 20; or
(ii) a CDR-H1 domain comprising SEQ ID No: 164, a CDR-H2 domain comprising SEQ ID No: 10, a CDR-H3 domain comprising SEQ ID No: 11, a CDR-L1 domain comprising SEQ ID No: 18, a CDR-L2 domain comprising SEQ ID No: 19 and/or a CDR-L3 domain comprising SEQ ID No: 20,
optionally wherein the antibody or antigen-binding fragment thereof comprises at least one, at least two, at least three, at least four, at least five, or at least six of the CDRs.

12. The antibody or antigen-binding fragment thereof according to any one of claims 9 to 11, wherein the antibody or antigen-binding fragment thereof comprises:
(i) a heavy chain variable region comprising or consisting of SEQ ID No: 16 and a light chain variable region comprising or consisting of SEQ ID No: 25;
(ii) a heavy chain variable region comprising or consisting of SEQ ID No: 156 and a light chain variable region comprising or consisting of SEQ ID No: 25;
(iii) a heavy chain variable region comprising or consisting of SEQ ID No: 165 and a light chain variable region comprising or consisting of SEQ ID No: 25;
(iv) a heavy chain variable region comprising or consisting of SEQ ID No: 172 and a light chain variable region comprising or consisting of SEQ ID No: 25;
(v) a heavy chain variable region comprising or consisting of SEQ ID No: 156 and a light chain variable region comprising or consisting of SEQ ID No: 190;
(vi) a heavy chain variable region comprising or consisting of SEQ ID No: 156 and a light chain variable region comprising or consisting of SEQ ID No: 191;
(vii) a heavy chain variable region comprising or consisting of SEQ ID No: 194 and a light chain variable region comprising or consisting of SEQ ID No: 25;
(viii) a heavy chain variable region comprising or consisting of SEQ ID No: 172 and a light chain variable region comprising or consisting of SEQ ID No: 191;
(ix) a heavy chain variable region comprising or consisting of SEQ ID No: 172 and a light chain variable region comprising or consisting of SEQ ID No: 190;
(x) a heavy chain variable region comprising or consisting of SEQ ID No: 194 and a light chain variable region comprising or consisting of SEQ ID No: 191;
(xi) a heavy chain variable region comprising or consisting of SEQ ID No: 194 and a light chain variable region comprising or consisting of SEQ ID No: 190;
(xii) a heavy chain variable region comprising or consisting of SEQ ID No: 165 and a light chain variable region comprising or consisting of SEQ ID No: 191; or
(xiii) a heavy chain variable region comprising or consisting of SEQ ID No: 165 and a light chain variable region comprising or consisting of SEQ ID No: 190.

13. The antibody or antigen-binding fragment thereof according to either claim 9 or claim 10, wherein the antibody or antigen-binding fragment thereof comprises:
(i) a CDR-H1 domain comprising SEQ ID No: 27, a CDR-H2 domain comprising SEQ ID No: 28, a CDR-H3 domain comprising SEQ ID No: 29, a CDR-L1 domain comprising SEQ ID No: 36, a CDR-L2 domain comprising SEQ ID No: 37 and/or a CDR-L3 domain comprising SEQ ID No: 38; or
(ii) a CDR-H1 domain comprising SEQ ID No: 164, a CDR-H2 domain comprising SEQ ID No: 28, a CDR-H3 domain comprising SEQ ID No: 29, a CDR-L1 domain comprising SEQ ID No: 36, a CDR-L2 domain comprising SEQ ID No: 37 and/or a CDR-L3 domain comprising SEQ ID No: 38,
optionally wherein the antibody or antigen-binding fragment thereof comprises at least one, at least two, at least three, at least four, at least five, or at least six of the CDRs.

14. The antibody or antigen-binding fragment thereof according to claim 9 or claim or claim 13, wherein the antibody or antigen-binding fragment thereof comprises:
(i) a heavy chain variable region comprising or consisting of SEQ ID No: 34 and a light chain variable region comprising or consisting of SEQ ID No: 43;
(ii) a heavy chain variable region comprising or consisting of SEQ ID No: 159 and a light chain variable region comprising or consisting of SEQ ID No: 43;
(iii) a heavy chain variable region comprising or consisting of SEQ ID No: 165 and a light chain variable region comprising or consisting of SEQ ID No: 43;
(iv) a heavy chain variable region comprising or consisting of SEQ ID No: 166 and a light chain variable region comprising or consisting of SEQ ID No: 43; 35
(v) a heavy chain variable region comprising or consisting of SEQ ID No: 167 and a light chain variable region comprising or consisting of SEQ ID No: 43;
(vi) a heavy chain variable region comprising or consisting of SEQ ID No: 168 and a light chain variable region comprising or consisting of SEQ ID No: 43;
(vii) a heavy chain variable region comprising or consisting of SEQ ID No: 169 and a light chain variable region comprising or consisting of SEQ ID No: 43;
(viii) a heavy chain variable region comprising or consisting of SEQ ID No: 170 and a light chain variable region comprising or consisting of SEQ ID No: 43;
(ix) a heavy chain variable region comprising or consisting of SEQ ID No: 171 and a light chain variable region comprising or consisting of SEQ ID No: 43;
(x) a heavy chain variable region comprising or consisting of SEQ ID No: 172 and a light chain variable region comprising or consisting of SEQ ID No: 43;
(xi) a heavy chain variable region comprising or consisting of SEQ ID No: 159 and a light chain variable region comprising or consisting of SEQ ID No: 185;
(xii) a heavy chain variable region comprising or consisting of SEQ ID No: 159 and a light chain variable region comprising or consisting of SEQ ID No: 186;
(xiii) a heavy chain variable region comprising or consisting of SEQ ID No: 194 and a light chain variable region comprising or consisting of SEQ ID No: 43;
(xiv) a heavy chain variable region comprising or consisting of SEQ ID No: 172 and a light chain variable region comprising or consisting of SEQ ID No: 186;
(xv) a heavy chain variable region comprising or consisting of SEQ ID No: 172 and a light chain variable region comprising or consisting of SEQ ID No: 185;
(xvi) a heavy chain variable region comprising or consisting of SEQ ID No: 171 and a light chain variable region comprising or consisting of SEQ ID No: 186;
(xvii) a heavy chain variable region comprising or consisting of SEQ ID No: 171 and a light chain variable region comprising or consisting of SEQ ID No: 185;
(xviii) a heavy chain variable region comprising or consisting of SEQ ID No: 170 and a light chain variable region comprising or consisting of SEQ ID No: 186;
(xix) a heavy chain variable region comprising or consisting of SEQ ID No: 170 and a light chain variable region comprising or consisting of SEQ ID No: 185;
(xx) a heavy chain variable region comprising or consisting of SEQ ID No: 169 and a light chain variable region comprising or consisting of SEQ ID No: 186;
(xxi) a heavy chain variable region comprising or consisting of SEQ ID No: 169 and a light chain variable region comprising or consisting of SEQ ID No: 185;
(xxii) a heavy chain variable region comprising or consisting of SEQ ID No: 168 and a light chain variable region comprising or consisting of SEQ ID No: 186;
(xxiii) a heavy chain variable region comprising or consisting of SEQ ID No: 168 and a light chain variable region comprising or consisting of SEQ ID No: 185;
(xxiv) a heavy chain variable region comprising or consisting of SEQ ID No: 167 and a light chain variable region comprising or consisting of SEQ ID No: 186;
(xxv) a heavy chain variable region comprising or consisting of SEQ ID No: 167 and a light chain variable region comprising or consisting of SEQ ID No: 185;
(xxvi) a heavy chain variable region comprising or consisting of SEQ ID No: 166 and a light chain variable region comprising or consisting of SEQ ID No: 186;
(xxvii) a heavy chain variable region comprising or consisting of SEQ ID No: 166 and a light chain variable region comprising or consisting of SEQ ID No: 185;
(xxviii) a heavy chain variable region comprising or consisting of SEQ ID No: 194 and a light chain variable region comprising or consisting of SEQ ID No: 186;
(xxix) a heavy chain variable region comprising or consisting of SEQ ID No: 194 and a light chain variable region comprising or consisting of SEQ ID No: 185;
(xxx) a heavy chain variable region comprising or consisting of SEQ ID No: 165 and a light chain variable region comprising or consisting of SEQ ID No: 186; or
(xxxi) a heavy chain variable region comprising or consisting of SEQ ID No: 168 and a light chain variable region comprising or consisting of SEQ ID No: 185.

15. The antibody or antigen-binding fragment thereof according to either claim 9 or claim 10, wherein the antibody or antigen-binding fragment thereof comprises
(i) a CDR-H1 domain comprising SEQ ID No: 45, a CDR-H2 domain comprising SEQ ID No: 46, a CDR-H3 domain comprising SEQ ID No: 47, a CDR-L1 domain comprising SEQ ID No: 54, a CDR-L2 domain comprising SEQ ID No: 55, and/or a CDR-L3 domain comprising SEQ ID No: 56; or
(ii) a CDR-H1 domain comprising SEQ ID No: 173, a CDR-H2 domain comprising SEQ ID No: 46, a CDR-H3 domain comprising SEQ ID No: 47, a CDR-L1 domain comprising SEQ ID No: 54, a CDR-L2 domain comprising SEQ ID No: 55, and/or a CDR-L3 domain comprising SEQ ID No: 56,
optionally wherein the antibody or antigen-binding fragment thereof comprises at least one, at least two, at least three, at least four, at least five, or at least six of the CDRs.

16. The antibody or antigen-binding fragment thereof according to claim 9 or claim or claim 15, wherein the antibody or antigen-binding fragment thereof comprises:
(i) a heavy chain variable region comprising or consisting of SEQ ID No: 52 and a light chain variable region comprising or consisting of SEQ ID No: 61;
(ii) a heavy chain variable region comprising or consisting of SEQ ID No: 174 and a light chain variable region comprising or consisting of SEQ ID No: 61;
(iii) a heavy chain variable region comprising or consisting of SEQ ID No: 175 and a light chain variable region comprising or consisting of SEQ ID No: 61;
(iv) a heavy chain variable region comprising or consisting of SEQ ID No: 176 and a light chain variable region comprising or consisting of SEQ ID No: 61;
(v) a heavy chain variable region comprising or consisting of SEQ ID No: 52 and a light chain variable region comprising or consisting of SEQ ID No: 187;
(vi) a heavy chain variable region comprising or consisting of SEQ ID No: 52 and a light chain variable region comprising or consisting of SEQ ID No: 188;
(vii) a heavy chain variable region comprising or consisting of SEQ ID No: 176 and a light chain variable region comprising or consisting of SEQ ID No: 188;
(viii) a heavy chain variable region comprising or consisting of SEQ ID No: 176 and a light chain variable region comprising or consisting of SEQ ID No: 187;
(ix) a heavy chain variable region comprising or consisting of SEQ ID No: 175 and a light chain variable region comprising or consisting of SEQ ID No: 188;
(x) a heavy chain variable region comprising or consisting of SEQ ID No: 175 and a light chain variable region comprising or consisting of SEQ ID No: 187; 20)
(xi) a heavy chain variable region comprising or consisting of SEQ ID No: 174 and a light chain variable region comprising or consisting of SEQ ID No: 188; or
(xii) a heavy chain variable region comprising or consisting of SEQ ID No: 174 and a light chain variable region comprising or consisting of SEQ ID No: 187.

17. The antibody or antigen-binding fragment thereof according to either claim 9 or claim 10, wherein the antibody or antigen-binding fragment thereof comprises:
(i) a CDR-H1 domain comprising SEQ ID No: 63, a CDR-H2 domain comprising SEQ ID No: 64, a CDR-H3 domain comprising SEQ ID No: 65, a CDR-L1 domain comprising SEQ ID No: 72, a CDR-L2 domain comprising SEQ ID No: 73, and/or a CDR-L3 domain comprising SEQ ID No: 74; or
(ii) a CDR-H1 domain comprising SEQ ID No: 177, a CDR-H2 domain comprising SEQ ID No: 64, a CDR-H3 domain comprising SEQ ID No: 65, a CDR-L1 domain comprising SEQ ID No: 72, a CDR-L2 domain comprising SEQ ID No: 73, and/or a CDR-L3 domain comprising SEQ ID No: 74,
optionally wherein the antibody or antigen-binding fragment thereof comprises at least one, at least two, at least three, at least four, at least five, or at least six of the CDRs.

18. The antibody or antigen-binding fragment thereof according to claim 9 or claim or claim 17, wherein the antibody or antigen-binding fragment thereof comprises:
(i) a heavy chain variable region comprising or consisting of SEQ ID No: 70 and a light chain variable region comprising or consisting of SEQ ID No: 79;
(ii) a heavy chain variable region comprising or consisting of SEQ ID No: 160 and a light chain variable region comprising or consisting of SEQ ID No: 79; 10)
(iii) a heavy chain variable region comprising or consisting of SEQ ID No: 178 and a light chain variable region comprising or consisting of SEQ ID No: 79;
(iv) a heavy chain variable region comprising or consisting of SEQ ID No: 179 and a light chain variable region comprising or consisting of SEQ ID No: 79;
(v) a heavy chain variable region comprising or consisting of SEQ ID No: 180 and a light chain variable region comprising or consisting of SEQ ID No: 79;
(vi) a heavy chain variable region comprising or consisting of SEQ ID No: 160 and a light chain variable region comprising or consisting of SEQ ID No: 189;
(vii) a heavy chain variable region comprising or consisting of SEQ ID No: 180 and a light chain variable region comprising or consisting of SEQ ID No: 189;
(viii) a heavy chain variable region comprising or consisting of SEQ ID No: 179 and a light chain variable region comprising or consisting of SEQ ID No: 189; or
(ix) a heavy chain variable region comprising or consisting of SEQ ID No: 178 and a light chain variable region comprising or consisting of SEQ ID No: 189.

19. The antibody or antigen-binding fragment thereof according to either claim 9 or claim 10, wherein the antibody or antigen-binding fragment thereof comprises:
(i) a CDR-H1 domain comprising SEQ ID No: 81, a CDR-H2 domain comprising SEQ ID No: 82, a CDR-H3 domain comprising SEQ ID No: 83, a CDR-L1 domain comprising SEQ ID No: 90, a CDR-L2 domain comprising SEQ ID No: 91, and/or a CDR-L3 domain comprising SEQ ID No: 92; or
(ii) a CDR-H1 domain comprising SEQ ID No: 181, a CDR-H2 domain comprising SEQ ID No: 82, a CDR-H3 domain comprising SEQ ID No: 83, a CDR-L1 domain comprising SEQ ID No: 90, a CDR-L2 domain comprising SEQ ID No: 91, and/or a CDR-L3 domain comprising SEQ ID No: 92,
optionally wherein the antibody or antigen-binding fragment thereof comprises at least one, at least two, at least three, at least four, at least five, or at least six of the CDRs.

20. The antibody or antigen-binding fragment thereof according to claim 9 or claim or claim 19, wherein the antibody or antigen-binding fragment thereof comprises:
(i) a heavy chain variable region comprising or consisting of SEQ ID No: 88 and a light chain variable region comprising or consisting of SEQ ID No: 97;
(ii) a heavy chain variable region comprising or consisting of SEQ ID No: 161 and a light chain variable region comprising or consisting of SEQ ID No: 97; 10
(iii) a heavy chain variable region comprising or consisting of SEQ ID No: 182 and a light chain variable region comprising or consisting of SEQ ID No: 97;
(iv) a heavy chain variable region comprising or consisting of SEQ ID No: 183 and a light chain variable region comprising or consisting of SEQ ID No: 97;
(v) a heavy chain variable region comprising or consisting of SEQ ID No: 184 and a light chain variable region comprising or consisting of SEQ ID No: 97;
(vi) a heavy chain variable region comprising or consisting of SEQ ID No: 161 and a light chain variable region comprising or consisting of SEQ ID No: 192;
(vii) a heavy chain variable region comprising or consisting of SEQ ID No: 161 and a light chain variable region comprising or consisting of SEQ ID No: 193;
(viii) a heavy chain variable region comprising or consisting of SEQ ID No: 184 and a light chain variable region comprising or consisting of SEQ ID No: 193;
(ix) a heavy chain variable region comprising or consisting of SEQ ID No: 184 and a light chain variable region comprising or consisting of SEQ ID No: 192;
(x) a heavy chain variable region comprising or consisting of SEQ ID No: 183 and a light chain variable region comprising or consisting of SEQ ID No: 193;
(xi) a heavy chain variable region comprising or consisting of SEQ ID No: 183 and a light chain variable region comprising or consisting of SEQ ID No: 192;
(xii) a heavy chain variable region comprising or consisting of SEQ ID No: 182 and a light chain variable region comprising or consisting of SEQ ID No: 193; or
(xiii) a heavy chain variable region comprising or consisting of SEQ ID No: 182 and a light chain variable region comprising or consisting of SEQ ID No: 192.

21. The antibody or antigen-binding fragment thereof according to either claim 9 or claim 10, wherein the antibody or antigen-binding fragment thereof comprises a CDR-H1 domain comprising SEQ ID No: 99, a CDR-H2 domain comprising SEQ ID No: 100, a CDR-H3 domain comprising SEQ ID No: 101, a CDR-L1 domain comprising SEQ ID No: 108, a CDR-L2 domain comprising SEQ ID No: 109, and/or a CDR-L3 domain comprising SEQ ID No: 110, optionally wherein the antibody or antigen-binding fragment thereof comprises at least one, at least two, at least three, at least four, at least five, or at least six of the CDRs.

22. The antibody or antigen-binding fragment thereof according to claim 9 or claim or claim 21, wherein the antibody or antigen-binding fragment thereof comprises a heavy chain variable region comprising or consisting of SEQ ID No: 106 and a light chain variable region comprising or consisting of SEQ ID No: 115.

23. The antibody or antigen-binding fragment thereof according to either claim 9 or claim 10, wherein the antibody or antigen-binding fragment thereof comprises a CDR-H1 domain comprising SEQ ID No: 117, a CDR-H2 domain comprising SEQ ID No: 118, a CDR-H3 domain comprising SEQ ID No: 119, a CDR-L1 domain comprising SEQ ID No: 126, a CDR-L2 domain comprising SEQ ID No: 127, and/or a CDR-L3 domain comprising SEQ ID No: 128, optionally wherein the antibody or antigen-binding fragment thereof comprises at least one, at least two, at least three, at least four, at least five, or at least six of the CDRs.

24. The antibody or antigen-binding fragment thereof according to claim 9 or claim or claim 23, wherein the antibody or antigen-binding fragment thereof comprises a heavy chain variable region comprising or consisting of SEQ ID No: 124 and a light chain variable region comprising or consisting of SEQ ID No: 133.

25. The antibody or antigen-binding fragment thereof according to either claim 9 or claim 10, wherein the antibody or antigen-binding fragment thereof comprises a CDR-H1 domain comprising SEQ ID No: 135, a CDR-H2 domain comprising SEQ ID No: 136, a CDR-H3 domain comprising SEQ ID No: 137, a CDR-L1 domain comprising SEQ ID No: 144, a CDR-L2 domain comprising SEQ ID No: 145, and/or a CDR-L3 domain comprising SEQ ID No: 146, optionally wherein the antibody or antigen-binding fragment thereof comprises at least one, at least two, at least three, at least four, at least five, or at least six of the CDRs.

26. The antibody or antigen-binding fragment thereof according to claim 9 or claim or claim 25, wherein the antibody or antigen-binding fragment thereof comprises a heavy chain variable region comprising or consisting of SEQ ID No: 142 and a light chain variable region comprising or consisting of SEQ ID No: 151.

27. An inhibitor according to any one of claims 1 to 8, or an antibody or an antigen-binding fragment thereof according to any one of claims 9 to 26, for use in therapy.

28. An inhibitor according to any one of claims 1 to 8, or an antibody or an antigen-binding fragment thereof according to any one of claims 9 to 26, for use in treating, preventing or ameliorating a condition caused by platelet-mediated aggregation.

29. An inhibitor, or an antibody or an antigen-binding fragment thereof for use according to claim 28, wherein the condition caused by platelet-mediated aggregation may be selected from the group consisting of: a thrombotic-related condition; thrombotic thrombocytopenia purpura (TTP); acquired thrombotic thrombocytopenic purpura (aTTP), acute coronary syndrome (ACS), atherosclerosis, ischemic stroke, atrial fibrillation (AF), acute myocardial infarction (AMI), cardiovascular disease (CVD), thrombosis, unstable angina, stable angina, angina pectoris, embolus formation, deep vein thrombosis, haemolytic uremic syndrome, haemolytic anaemia, acute renal failure, thrombolytic complications, disseminated intravascular coagulation, coronary heart disease, thromboembolic complications, restenosis, chronic unstable angina, peripheral vascular disease, arterial thrombosis, pre-eclampsia, embolism, restenosis, sepsis, vascular inflammation, glomerulonephritis, and thrombotic condition resulting from a coronavirus infection.

30. A pharmaceutical composition comprising an inhibitor according to any one of claims 1 to 8, or an antibody or antigen-binding fragment thereof according to any one of claims 9 to 26, and optionally a pharmaceutically acceptable vehicle.

31. An antibody or antigen-binding fragment thereof obtained by a method comprising:—
(i) immunising a host organism with one or more of a C1, C2, C3, C4, C5, and/or C6 domain of Von Willebrand Factor (VWF); and
(ii) collecting an antibody or antigen-binding fragment thereof from the host.

32. A polynucleotide sequence encoding the antibody, or antigen-binding fragment thereof as defined in any one of claims 9 to 26.

33. An expression cassette comprising a polynucleotide sequence according to claim 32.

34. A recombinant vector comprising the expression cassette according to claim 33.

35. A host cell comprising the polynucleotide sequence according to claim 32, the expression cassette according to claim 33, or the vector according to claim 34.

36. A method of preparing the antibody or antigen binding fragment according to any one of claims 9 to 26, the method comprising:
a) introducing, into a host cell, the vector of claim 34; and
b) culturing the host cell under conditions to result in the production of the antibody or antigen binding fragment according to any one of claims 9 to 26.

37. The antibody or antibody binding fragment thereof according to any one of claims 9 to 26, for use in diagnosis or prognosis.

38. The antibody or antibody binding fragment thereof according to any one of claims 9 to 26, for use in diagnosing or prognosing a condition caused by platelet-mediated aggregation.

39. A method of diagnosing or prognosing a condition caused by platelet-mediated aggregation in a subject, the method comprising detecting VWF in a biological sample obtained from the subject with the antibody or antibody binding fragment thereof according to any one of claims 9 to 26.

40. A kit for diagnosing a subject suffering from a condition caused by platelet-mediated aggregation, or for providing a prognosis of the subject's condition, the kit comprising an antibody or antigen-binding fragment thereof according to any one of claims 9 to 26 for detecting VWF in a sample from a test subject.

41. The use according to claim 38, the method according to claim 39, or the kit according to claim 40, wherein the condition caused by platelet-mediated aggregation may be selected from the group consisting of: a thrombotic-related condition; thrombotic thrombocytopenia purpura (TTP); acquired thrombotic thrombocytopenia purpura (aTTP), acute coronary syndrome (ACS), atherosclerosis, ischemic stroke, atrial fibrillation (AF), acute myocardial infarction (AMI), cardiovascular disease (CVD), thrombosis, unstable angina, stable angina, angina pectoris, embolus formation, deep vein thrombosis, haemolytic uremic syndrome, haemolytic anaemia, acute renal failure, thrombolytic complications, disseminated intravascular coagulation, coronary heart disease, thromboembolic complications, restenosis, chronic unstable angina, peripheral vascular disease, arterial thrombosis, pre-eclampsia, embolism, restenosis, sepsis, vascular inflammation, glomerulonephritis, and thrombotic condition resulting from a coronavirus infection.
